(19) United States
(12) Patent Application Publication
(10) Pub. No.: US 2024/0343715 A1
(43) Pub. Date: Oct. 17, 2024

(54) ORGANIC COMPOUNDS, MIXTURES AND FORMULATIONS COMPRISING SAME, ORGANIC ELECTRONIC DEVICES, AND APPLICATIONS OF COMPOUNDS

(71) Applicant: Zhejiang Brilliant Optoelectronic Technology Co.,Ltd., Taizhou (CN)

(72) Inventors: Jiahui TAN, Taizhou (CN); Hao ZHANG, Taizhou (CN); Junyou PAN, Taizhou (CN)

(73) Assignee: Zhejiang Brilliant Optoelectronic Technology Co.,Ltd., Taizhou (CN)

(21) Appl. No.: 18/756,237

(22) Filed: Jun. 27, 2024

Related U.S. Application Data
(63) Continuation of application No. PCT/CN2022/142222, filed on Dec. 27, 2022.

(30) Foreign Application Priority Data
Dec. 27, 2021 (CN) 202111613509.X

(51) Int. Cl. C07D 405/14; C07D 409/14; C09K 11/06; H10K 85/60; H10K 101/30

(52) U.S. Cl. CPC C07D 405/14; C07D 409/14; C09K 11/06; H10K 85/654; H10K 85/6572; H10K 85/6574; H10K 85/6576; H10K 2101/30

(57) ABSTRACT

Disclosed are organic compounds including a structure of formula (I). Also provided are mixtures containing a first organic compound (H1) and a second organic compound (H2), the first organic compound (H1) includes at least one of the organic compound. Further provided are organic electronic devices containing the organic compounds or the mixtures. By means of device structure optimization, improved device performance can be achieved, specifically, a high-performance OLED device can be implemented, improved material and manufacturing technical options are provided for full-color display and lighting applications.

### CROSS-REFERENCE TO RELATED APPLICATIONS

The present application is a continuation of International Application No. PCT/CN2022/142222, filed on Dec. 27, 2022, which claims priority to Chinese Patent Application No. 202111613509.X, filed on Dec. 27, 2021. All of the aforementioned applications are incorporated herein by reference in their entireties.

### TECHNICAL FIELD

The present disclosure relates to the field of organic electronic material and device technology, and in particular to an organic compound and a mixture thereof. The present disclosure also relates to an electronic device comprising the organic compound, in particular to electroluminescent devices, and the applications thereof.

### BACKGROUND

Due to the structural diversity, the low manufacturing cost, and the excellent optical and electrical properties, the organic light-emitting diodes (OLEDs) show great potential for optoelectronic device applications, such as flat-panel displays and lighting.

In order to improve the light-emitting properties of organic light-emitting diodes as well as to promote their large-scale industrialization, various organic optoelectronic material systems have been extensively developed. However, the OLED properties, especially the lifetime still requires further improvement. Therefore, new efficient and stable organic optoelectronic materials need to be developed urgently.

From the molecular aspect, the aggregation of organic molecules easily forms nonradiative transition and fluorescence quenching of excitons; from the structural aspect, electron-deficient group takes a nitrogen-containing heteroaromatic ring as an example, the nitrogen-containing heteroaromatic ring has good planarity, but poor structural stability, which greatly affects the processability of the optoelectronic materials and the property and lifetime of the optoelectronic devices. Therefore, appropriate spatial modification and protection of the electron-deficient groups of the organic optoelectronic molecule will be beneficial for improving the stability and optoelectronic property of such molecule. However, there is still limited research on related materials. Pat. No. CN104541576A discloses a class of triazine or pyrimidine derivatives, but the obtained device property, especially the lifetime, needs to be further improved.

In order to meet practical requirements, new molecular structures with high property have yet to be developed.

### SUMMARY

In one aspect, the present disclosure provides an organic compound comprising a structure of formula (I):

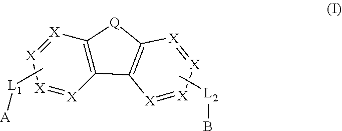

Where Q is O or S; X may be the same or different at each occurrence, and denote CR₁ or N, where no more than two Xs in each ring are N atoms, preferably no more than one X in each ring is N atom;

A comprises a structure of formula (A), B comprises a structure of formula (B):

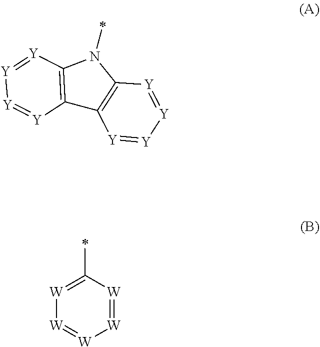

Where * denotes a linkage site; Y may be the same or different at each occurrence, and denote CR₂ or N, where no more than two Ys in each ring are N atoms, or the adjacent two Ys may be groups represented by formula (I-1) and formula (I-2), respectively, while the remaining Ys may be the same or different at each occurrence, and denote CR₁ or N;

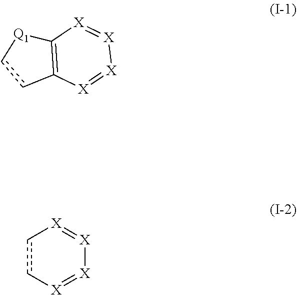

Where each dashed bond denotes a bonding position of the group (i.e., the position of Y=Y or Y-Y in formula (A)), each X is defined as described herein, Q₁ is selected from O, S, NR₂, or CR₃R₄;

Ws are the same or different and can independently be CR₅ or N, and at least one W is C—CN while at least two Ws are N atoms;

L₁ is a single bond, or a substituted/unsubstituted aromatic or heteroaromatic group containing 5 to 30 ring atoms; preferably, L₁ is a single bond, or a substituted/unsubstituted aromatic or heteroaromatic group containing 6 to 30 ring atoms;

L₂ is a single bond, or a substituted/unsubstituted aromatic or heteroaromatic group containing 5 to 30 ring atoms; preferably, L₂ is a single bond, or a substituted/unsubstituted aromatic or heteroaromatic group containing 6 to 30 ring atoms.

R₁ to R₅ may be same or different at each occurrence, and may independently selected from the group consisting of —H, —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, where one or more R₁-R₅ may form a monocyclic or polycyclic aliphatic or aromatic ring system with each other and/or with the rings bonded thereto.

In another aspect, the present disclosure also provides a polymer comprising at least one repeating unit, where at least one of the repeating unit of formula (I).

In yet another aspect, the present disclosure further provides a mixture comprising a first organic compound (H1) and a second organic compound (H2), where the first organic compound (H1) comprises at least one of the organic compound as described herein, the second organic compound (H2) has hole-transport property, and the molar ratio of the first organic compound (H1) to the second organic compound (H2) ranges from 1:9 to 9:1.

In yet another aspect, the present disclosure further provides a formulation comprising an organic compound or a polymer or a mixture as described herein, and at least one organic solvent.

In yet another aspect, the present disclosure further provides an organic electronic device comprising an organic compound or a polymer or a mixture as described herein.

Beneficial effect: the organic compound as described herein are used in OLEDs, in particular as a light-emitting layer material, which can provide high luminescence stability and long device lifetime.

### DETAILED DESCRIPTION OF THE EMBODIMENTS

The present disclosure is to provide an organic compound, a mixture, and the applications thereof in organic electronic devices, aiming to solve the problems of property and lifetime of the existing organic electronic devices. In order to make the objects, the technical solutions and the effects of the present disclosure more clear and definite, the present disclosure is further described in detail below. It should be understood that the embodiments described herein are only intended to explain the present disclosure and are not intended to limit the present disclosure.

As used herein, the terms “formulation”, “printing ink”, and “ink” have the same meaning, and they are interchangeable with each other.

As used herein, the terms “host material” and “matrix material” have the same meaning, and they are interchangeable with each other.

As used herein, the terms “metal organic clathrate”, “metal organic complexe”, and “organomentallic complexe” have the same meaning, and they are interchangeable with each other.

In one aspect, the present disclosure provides an organic compound comprising a structure of formula (I):

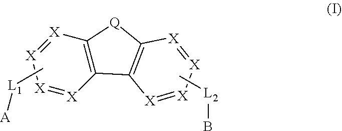

Where Q is O or S; X may be the same or different at each occurrence, and denote CR₁ or N, where no more than two Xs in each ring are N atoms, preferably no more than one X in each ring is N atom;

A comprises a structure of formula (A), B comprises a structure of formula (B):

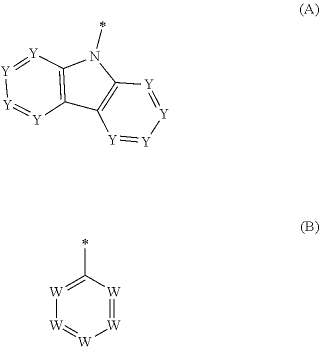

Where * denotes a linkage site; Y may be the same or different at each occurrence, and denote CR₂ or N, where no more than two Ys in each ring are N atoms, or the adjacent two Ys may be groups represented by formula (I-1) and formula (I-2), respectively, while the remaining Ys may be the same or different at each occurrence, and denote CR₁ or N;

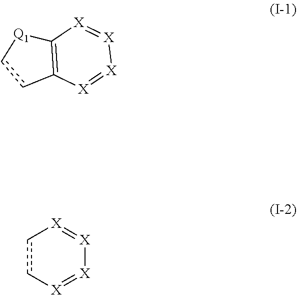

Where each dashed bond denotes a bonding position of the group (i.e., the position of Y=Y or Y-Y in formula (A)), each X is defined as described herein, Q₁ is selected from O, S, NR₂, or CR₃R₄; Ws are the same or different and can independently be CR₅ or N, and at least one W is C—CN while at least two Ws are N atoms; L₁ is a single bond, or a substituted/unsubstituted aromatic or heteroaromatic group containing 5 to 30 ring atoms; preferably, L₁ is a single bond, or a substituted/unsubstituted aromatic or heteroaromatic group containing 6 to 30 ring atoms;

L₂ is a single bond, or a substituted/unsubstituted aromatic or heteroaromatic group containing 5 to 30 ring atoms; preferably, L₂ is a single bond, or a substituted/unsubstituted aromatic or heteroaromatic group containing 6 to 30 ring atoms.

R₁ to R₅ may be same or different at each occurrence, and may independently selected from the group consisting of —H, —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, where one or more R₁-R₅ may form a monocyclic or polycyclic aliphatic or aromatic ring system with each other and/or with the rings bonded thereto.

The term “aromatic ring system” or “aromatic group” refers to a hydrocarbon group containing an aromatic ring, including monocyclic groups and polycyclic systems. The term “heteroaromatic ring system” or “heteroaromatic group” refers to a heteroaromatic group consisting of at least one hydrocarbon group (containing a heteroatom) consisting of at least one heteroaromatic ring, including monocyclic groups and polycyclic systems. The heteroatoms are preferably selected from Si, N, P, O, S and/or Ge, particularly preferably selected from Si, N, P, O and/or S. The polycyclic systems contain two or more rings, in which two carbon atoms are shared by two adjacent rings, i.e., fused rings. Specifically, at least one of the rings in the polycyclic rings are aromatic or heteroaromatic. For the purposes of the present disclosure, the aromatic or heteroaromatic ring systems contain not only aromatic or heteroaromatic systems, but also have a plurality of aryl or heteroaryl groups linked by short non-aromatic units (<10% of non-H atoms, preferably <5% of non-H atoms, such as C, N or O atoms). Therefore, a system such as 9,9′-spirobifluorene, 9,9-diarylfluorene, triarylamine, diaryl ether, and the like is also considered to be aromatic ring systems for the purposes of this disclosure.

Specifically, examples of the aromatic groups include, but not limited to benzene, naphthalene, anthracene, phenanthrene, perylene, tetracene, pyrene, benzopyrene, triphenylene, acenaphthylene, fluorene, and derivatives thereof.

Specifically, examples of heteroaromatic groups include, but not limited to furan, benzofuran, thiophene, benzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, o-diazanaphthalene, quinoxaline, phenanthridine, pyrimidine, quinazoline, quinazolinone, and derivatives thereof.

In some embodiments, A is selected from the following structures:

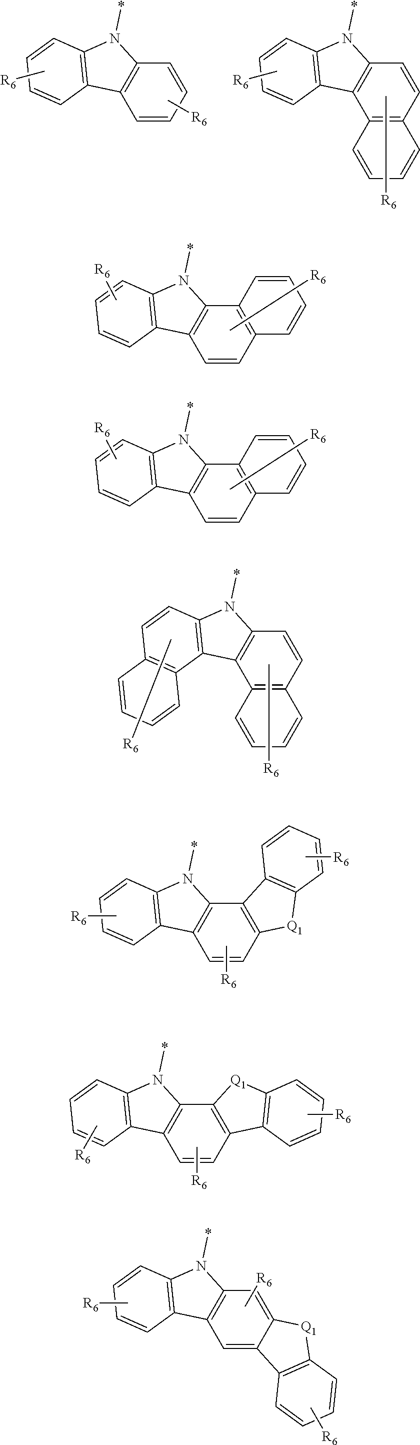

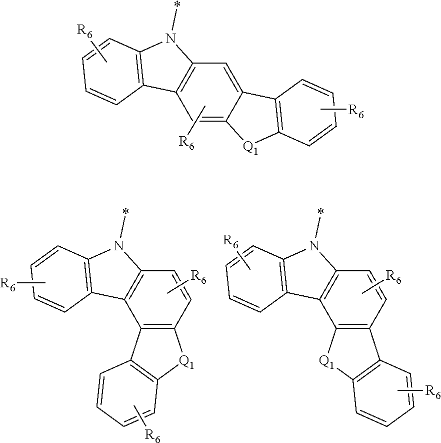

Where Q₁ is defined as described herein;

each R₆ is a substituent, and at each occurrence is independently selected from the group consisting of —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, where one or more R₆s may form a monocyclic or polycyclic aliphatic or aromatic ring system with each other and/or with the rings bonded thereto.

In some embodiments, A is selected from the following structures:

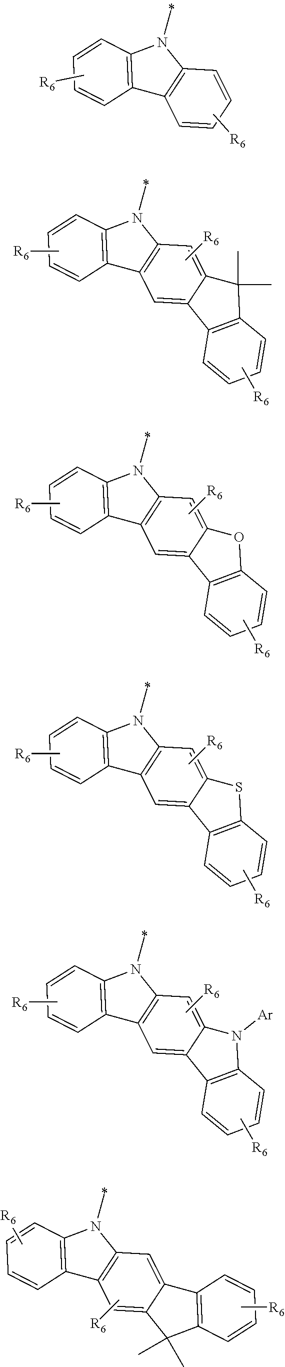

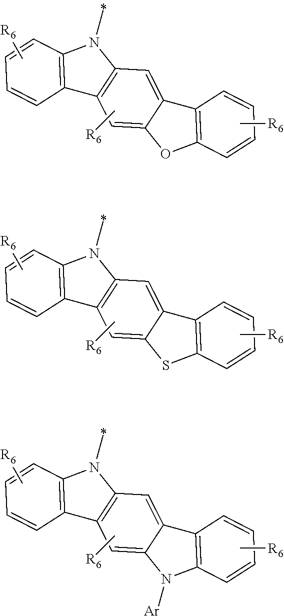

Where each Ar is independently selected from a substituted/unsubstituted aromatic or heteroaromatic ring system containing 5 to 40 ring atoms, an aryloxy or heteroaryloxy group containing 5 to 40 ring atoms, or any combination thereof, where one or more Ars may form a monocyclic or polycyclic aliphatic or aromatic ring system with each other and/or with the rings bonded thereto. R₆ is defined as described herein.

In some embodiments, each Ar is independently selected from a substituted/unsubstituted aromatic or heteroaromatic group containing 5 to 20 ring atoms, a deuterated/undeuterated aryloxy or heteroaryloxy group containing 5 to 20 ring atoms, or any combination thereof, where one or more Ars may form a monocyclic or polycyclic aliphatic or aromatic ring system with each other and/or with the rings bonded thereto.

In some embodiments, each Ar is independently selected from a substituted/unsubstituted aromatic or heteroaromatic group containing 5 to 15 ring atoms, a deuterated/undeuterated aryloxy or heteroaryloxy group containing 5 to 15 ring atoms, or any combination thereof, where one or more Ars may form a monocyclic or polycyclic aliphatic or aromatic ring system with each other and/or with the rings bonded thereto.

In some embodiments, each Ar is selected from biphenyl, naphthalene, anthracene, phenanthrene, pyrene, pyridine, pyrimidine, triazine, fluorene, silafluorene, carbazole, dibenzothiophene, dibenzofuran, triphenylamine, triphenyl phosphorus oxychloride, tetraphenylsilicon, spirofluorene, spirobifluorene, etc., and more preferably selected from biphenyl, naphthalene, fluorene, carbazole, dibenzothiophene, dibenzofuran, etc.

In some embodiments, each Ar is dibiphenyl.

In some embodiments, each Ar is benzene.

In some embodiments, the organic compound is selected from the following structures of formulas (II-1)-(II-14):

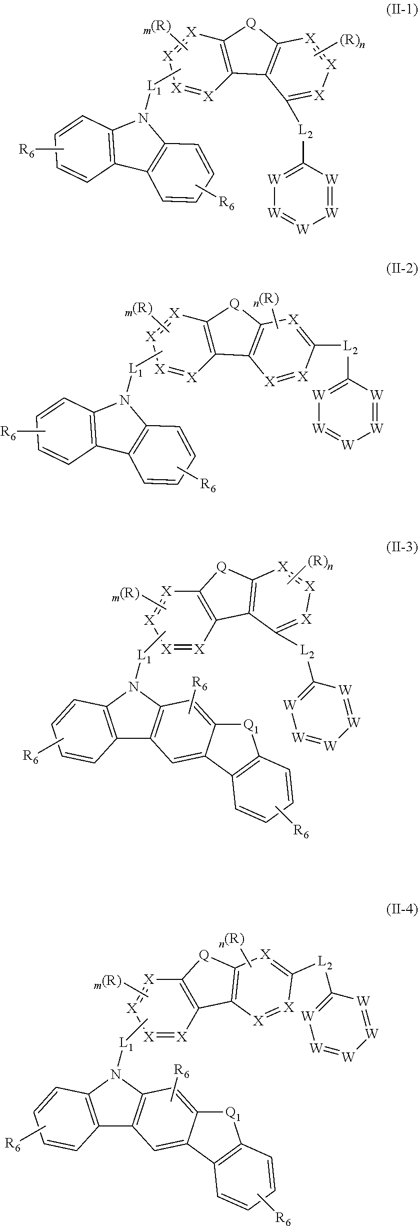

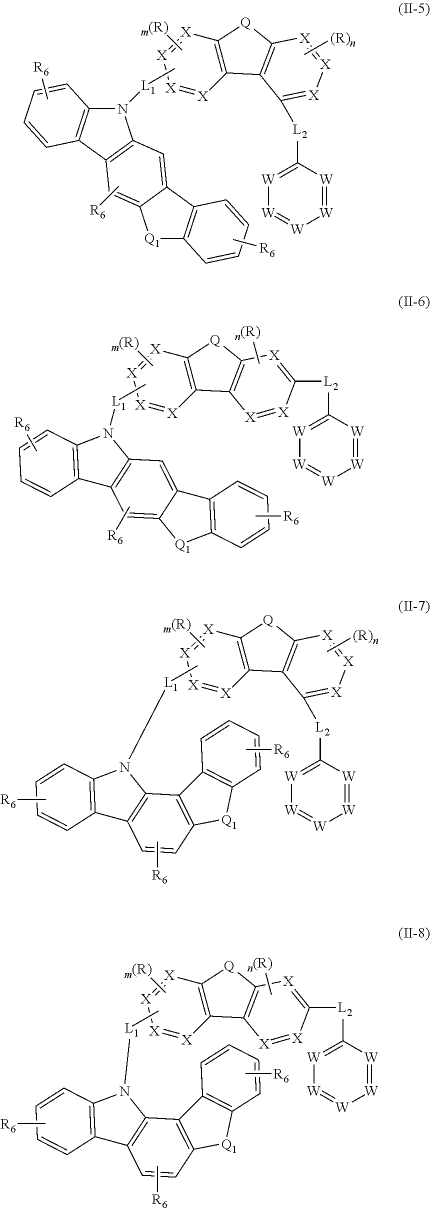

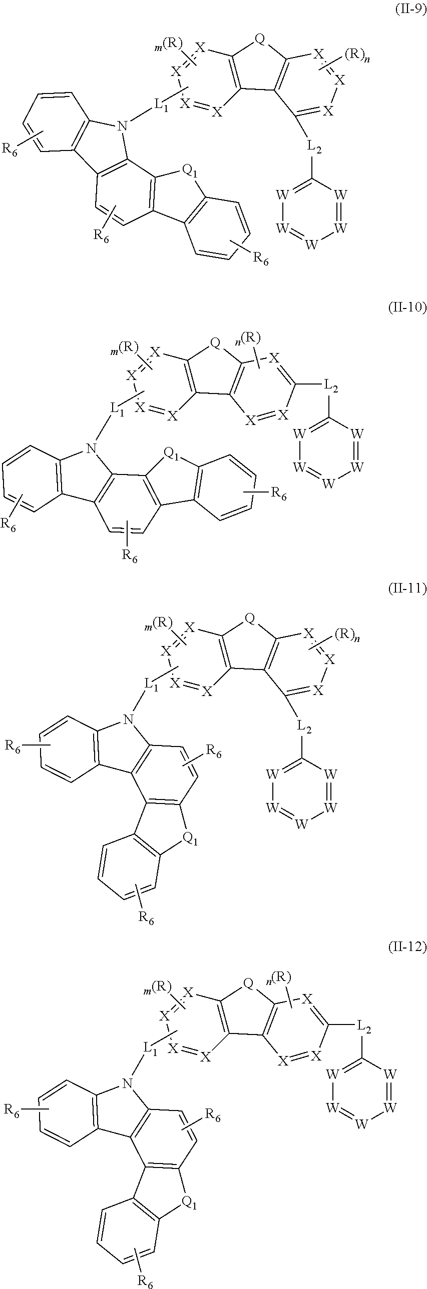

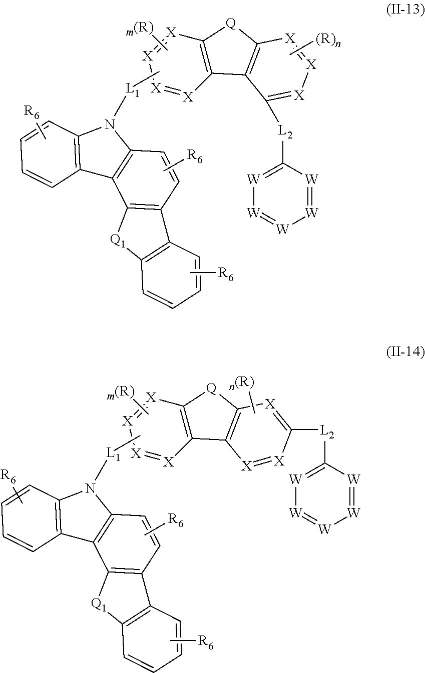

Where m, n are integers from 0 to 3, R is defined as R₆; X, Y, W, Q, Q₁, L₁, and L₂ are identically defined as described herein.

Preferably, each of L₁ and L₂ is independently selected from a single bond, or a substituted/unsubstituted aromatic or heteroaromatic or aryloxy or heteroaryloxy group containing 5 to 40 ring atoms, or any combination thereof, where one or more L₁-L₂ may form a monocyclic or polycyclic aliphatic or aromatic ring system with each other and/or with the rings bonded thereto.

In some embodiments, each of L₁ and L₂ is independently selected from the following groups or any combination thereof:

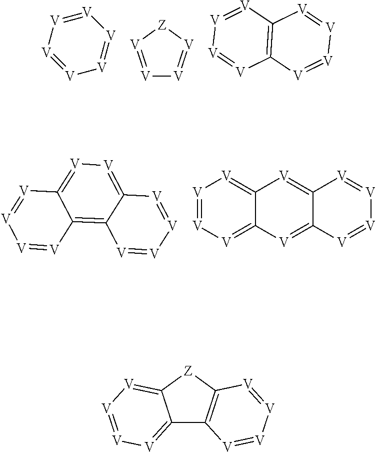

Where V at each occurrence is CR¹ or N; Z at each occurrence is independently selected from NR₇, CR₈R₉, O, S, SiR₁₀R₁₁, S═O, or SO₂; R¹ and R₇-R₁₁ are identically defined as the above-mentioned R₆.

In some embodiments, each of L₁ and L₂ is independently selected from the following groups or any combination thereof:

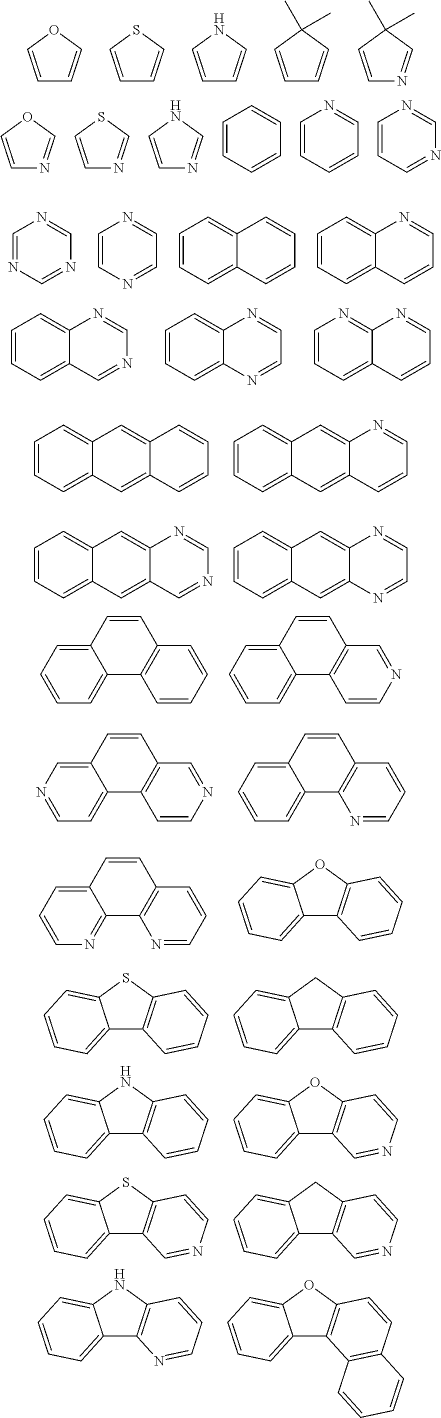

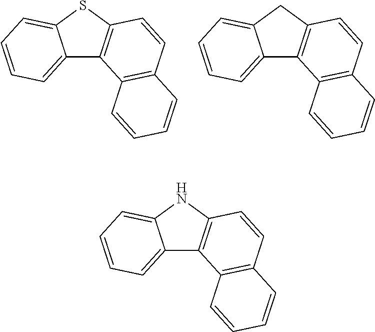

Where the H atoms on the ring may be further substituted.

In some embodiments, L₁ is a single bond.

The organic compound as described herein can be used as an organic functional material in electronic devices. The organic functional material is selected from a hole-injection material (HIM), a hole-transport material (HTM), an electron-transport material (ETM), an electron-injection material (EIM), an electron-blocking material (EBM), a hole-blocking material (HBM), an emitting material (Emitter), or a host material (Host). In some embodiments, the organic compound as described herein may be used as a host material or an electron-transport material. In some embodiments, the organic compound as described herein may be used as a phosphorescent host material.

As a phosphorescent host material, the organic compound should have an appropriate triplet energy level, namely T1. In some embodiments, the T1 of the organic compound ≥2.3 eV, preferably ≥2.4 eV, more preferably ≥2.5 eV, and most preferably ≥2.6 eV.

As a phosphorescent host material, it is desirable to have good thermal stability. Generally, the glass transition temperature (Tg) of the organic compound ≥100° C.; in some embodiments, Tg≥120° C.; in some embodiments, Tg≥140° C.; in some embodiments, Tg≥160° C.; in some embodiments, Tg≥180° C.

In some embodiments, the organic compound as described herein has a small singlet-triplet energy level difference (ΔEst), preferably ΔEst<0.3 eV, more preferably ΔEst<0.2 eV, further preferably ΔEst<0.15 eV, particularly preferably ΔEst<0.10 eV, and most preferably ΔEst<0.08 eV.

In some embodiments, the (HOMO−(HOMO−1)) of the organic compound ≥0.2 eV, preferably ≥0.25 eV, more preferably ≥0.3 eV, further preferably ≥0.35 eV, particularly preferably ≥0.4 eV, and most preferably ≥0.45 eV.

In some embodiments, the ((LUMO+1)−LUMO) of the organic compound ≥0.2 eV, preferably ≥0.25 eV, and most preferably ≥0.3 eV.

In some embodiments, the organic compound as described herein is partially deuterated; preferably 10% or more of total H, more preferably 20% or more of total H, further preferably 30% or more of total H, and most preferably 40% or more of total H, are deuterated.

In some embodiments, the organic compound as described herein is used for evaporation-based OLEDs. For this purpose, the molecular weight of the organic compound ≤1000 g/mol, preferably ≤900 g/mol, more preferably ≤850 g/mol, further preferably ≤800 g/mol, and most preferably ≤700 g/mol.

Specific examples of the organic compounds of formula (I) as described herein are listed below, but not limited thereto:

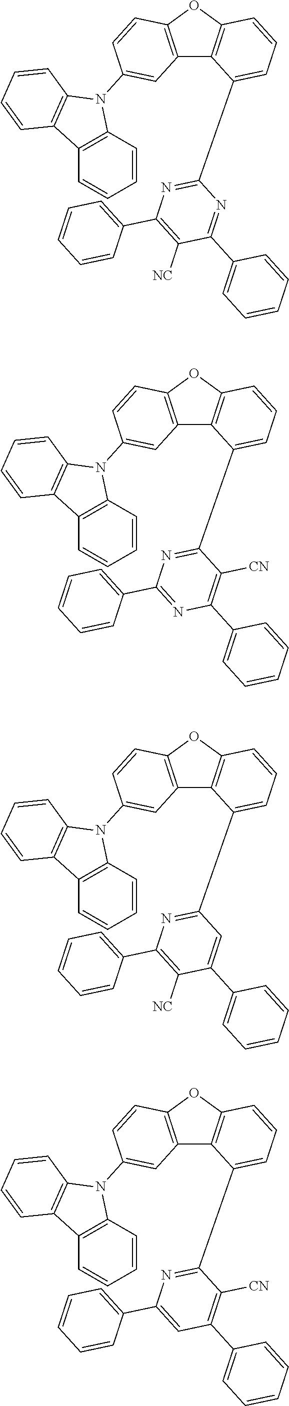 [SMILES: N#Cc1c(-c2ccccc2)nc(-c2cccc3oc4ccc(-n5c6ccccc6c6ccccc65)cc4c23)nc1-c1ccccc1 ; N#Cc1c(-c2ccccc2)nc(-c2ccccc2)nc1-c1cccc2oc3ccc(-n4c5ccccc5c5ccccc54)cc3c12 ; N#Cc1c(-c2ccccc2)cc(-c2cccc3oc4ccc(-n5c6ccccc6c6ccccc65)cc4c23)nc1-c1ccccc1 ; N#Cc1c(-c2ccccc2)cc(-c2ccccc2)nc1-c1cccc2oc3ccc(-n4c5ccccc5c5ccccc54)cc3c12]

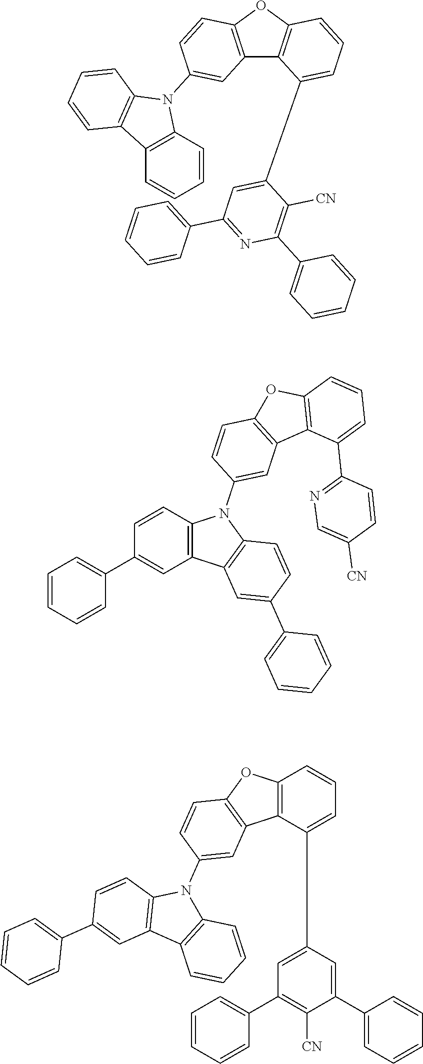 [SMILES: N#Cc1c(-c2cccc3oc4ccc(-n5c6ccccc6c6ccccc65)cc4c23)cc(-c2ccccc2)nc1-c1ccccc1 ; N#Cc1ccc(-c2cccc3oc4ccc(-n5c6ccc(-c7ccccc7)cc6c6cc(-c7ccccc7)ccc65)cc4c23)nc1 ; N#Cc1c(-c2ccccc2)cc(-c2cccc3oc4ccc(-n5c6ccccc6c6cc(-c7ccccc7)ccc65)cc4c23)cc1-c1ccccc1]

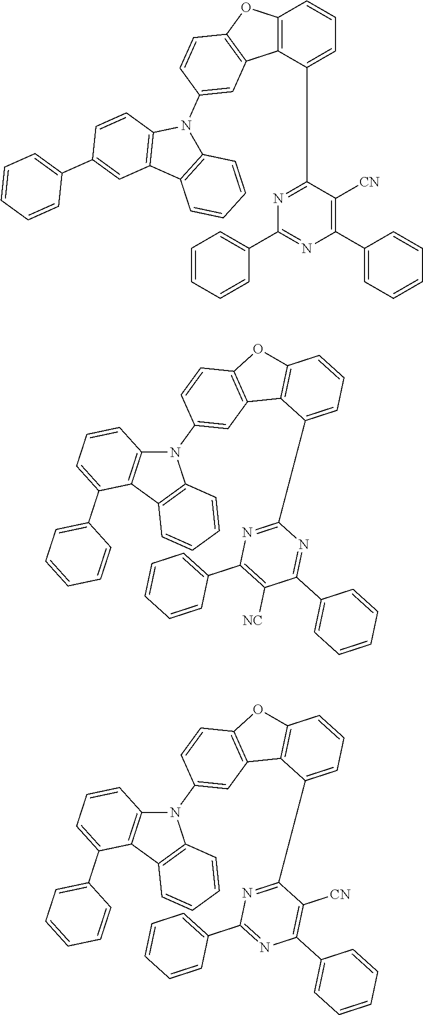 [SMILES: N#Cc1c(-c2ccccc2)nc(-c2ccccc2)nc1-c1cccc2oc3ccc(-n4c5ccccc5c5cc(-c6ccccc6)ccc54)cc3c12 ; N#Cc1c(-c2ccccc2)nc(-c2cccc3oc4ccc(-n5c6ccccc6c6c(-c7ccccc7)cccc65)cc4c23)nc1-c1ccccc1 ; N#Cc1c(-c2ccccc2)nc(-c2ccccc2)nc1-c1cccc2oc3ccc(-n4c5ccccc5c5c(-c6ccccc6)cccc54)cc3c12]

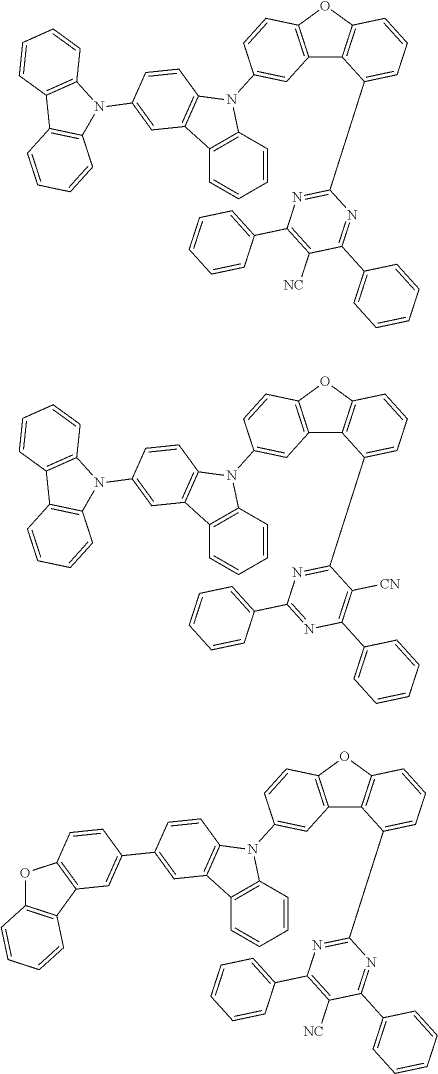 [SMILES: N#Cc1c(-c2ccccc2)nc(-c2cccc3oc4ccc(-n5c6ccccc6c6cc(-n7c8ccccc8c8ccccc87)ccc65)cc4c23)nc1-c1ccccc1 ; N#Cc1c(-c2ccccc2)nc(-c2ccccc2)nc1-c1cccc2oc3ccc(-n4c5ccccc5c5cc(-n6c7ccccc7c7ccccc76)ccc54)cc3c12 ; N#Cc1c(-c2ccccc2)nc(-c2cccc3oc4ccc(-n5c6ccccc6c6cc(-c7ccc8oc9ccccc9c8c7)ccc65)cc4c23)nc1-c1ccccc1]

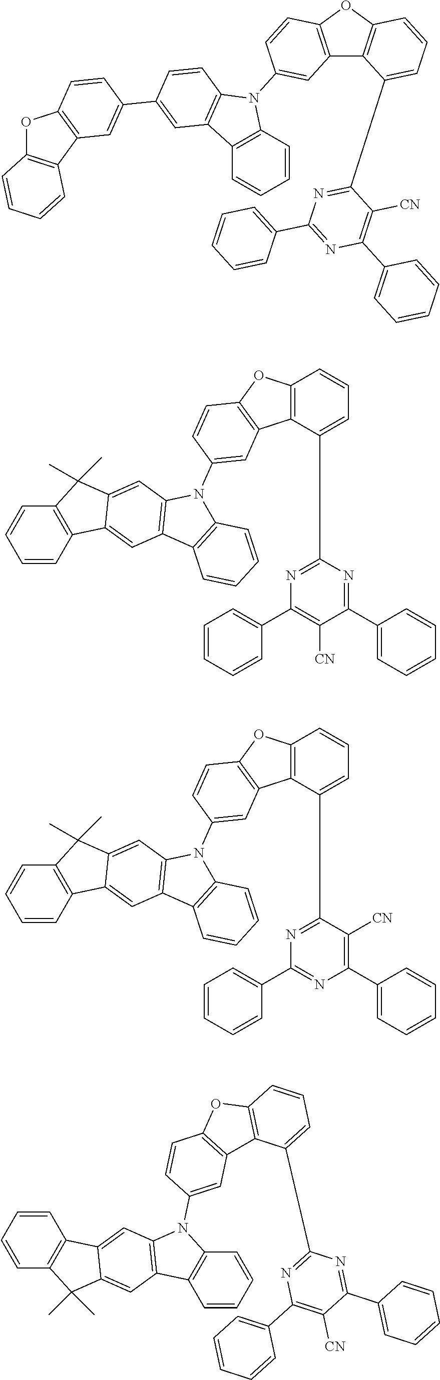 [SMILES: N#Cc1c(-c2ccccc2)nc(-c2ccccc2)nc1-c1cccc2oc3ccc(-n4c5ccccc5c5cc(-c6ccc7oc8ccccc8c7c6)ccc54)cc3c12 ; CC1(C)c2ccccc2-c2cc3c4ccccc4n(-c4ccc5oc6cccc(-c7nc(-c8ccccc8)c(C#N)c(-c8ccccc8)n7)c6c5c4)c3cc21 ; CC1(C)c2ccccc2-c2cc3c4ccccc4n(-c4ccc5oc6cccc(-c7nc(-c8ccccc8)nc(-c8ccccc8)c7C#N)c6c5c4)c3cc21 ; CC1(C)c2ccccc2-c2cc3c(cc21)c1ccccc1n3-c1ccc2oc3cccc(-c4nc(-c5ccccc5)c(C#N)c(-c5ccccc5)n4)c3c2c1]

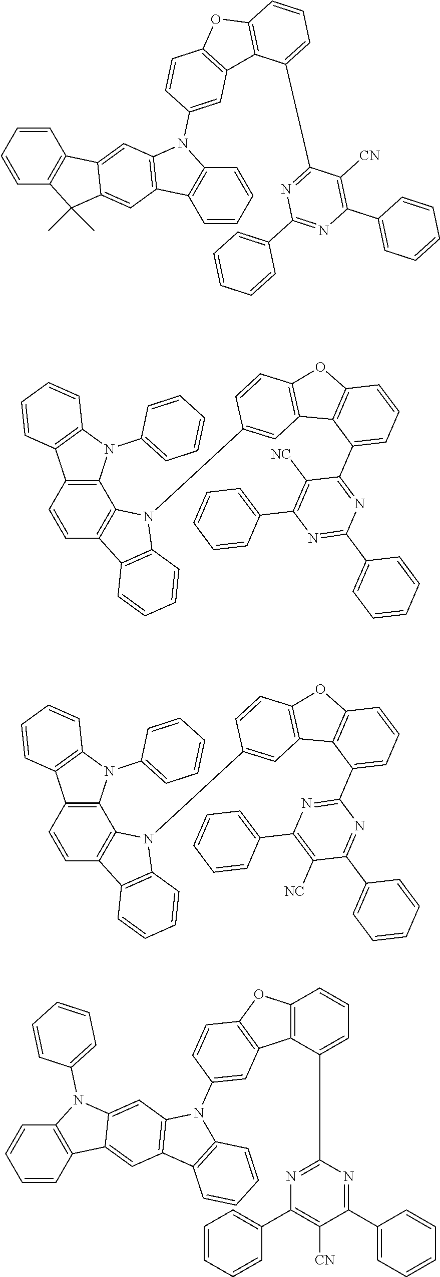 [SMILES: CC1(C)c2ccccc2-c2cc3c(cc21)c1ccccc1n3-c1ccc2oc3cccc(-c4nc(-c5ccccc5)nc(-c5ccccc5)c4C#N)c3c2c1 ; N#Cc1c(-c2ccccc2)nc(-c2ccccc2)nc1-c1cccc2oc3ccc(-n4c5ccccc5c5ccc6c7ccccc7n(-c7ccccc7)c6c54)cc3c12 ; N#Cc1c(-c2ccccc2)nc(-c2cccc3oc4ccc(-n5c6ccccc6c6ccc7c8ccccc8n(-c8ccccc8)c7c65)cc4c23)nc1-c1ccccc1 ; N#Cc1c(-c2ccccc2)nc(-c2cccc3oc4ccc(-n5c6ccccc6c6cc7c8ccccc8n(-c8ccccc8)c7cc65)cc4c23)nc1-c1ccccc1]

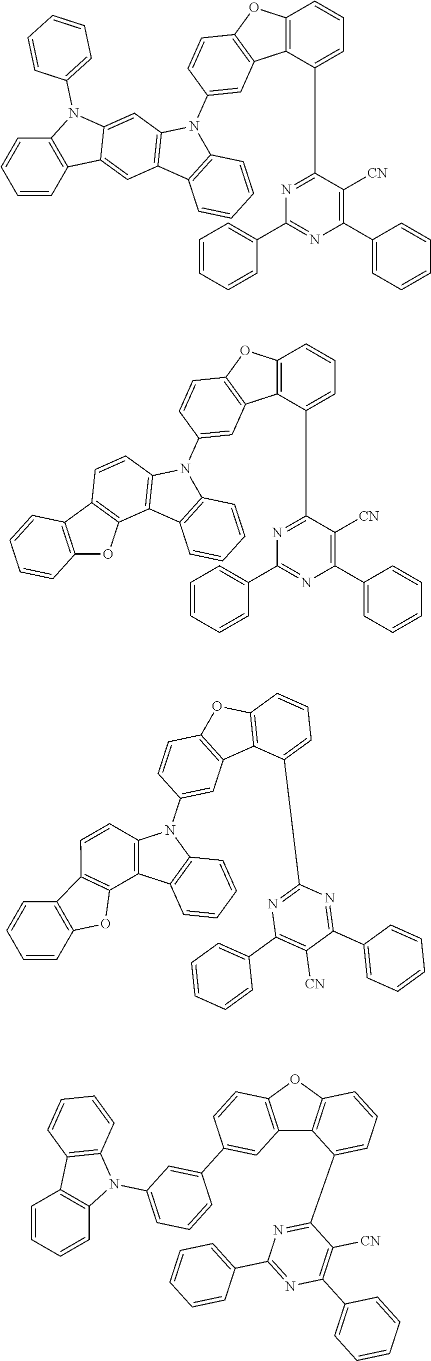 [SMILES: N#Cc1c(-c2ccccc2)nc(-c2ccccc2)nc1-c1cccc2oc3ccc(-n4c5ccccc5c5cc6c7ccccc7n(-c7ccccc7)c6cc54)cc3c12 ; N#Cc1c(-c2ccccc2)nc(-c2ccccc2)nc1-c1cccc2oc3ccc(-n4c5ccccc5c5c6oc7ccccc7c6ccc54)cc3c12 ; N#Cc1c(-c2ccccc2)nc(-c2cccc3oc4ccc(-n5c6ccccc6c6c7oc8ccccc8c7ccc65)cc4c23)nc1-c1ccccc1 ; N#Cc1c(-c2ccccc2)nc(-c2ccccc2)nc1-c1cccc2oc3ccc(-c4cccc(-n5c6ccccc6c6ccccc65)c4)cc3c12]

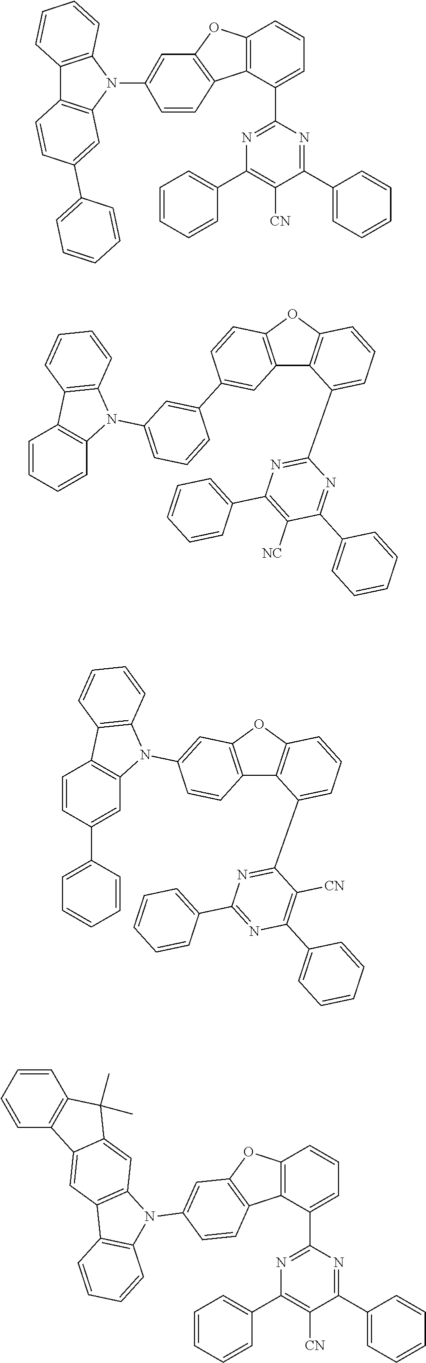 [SMILES: N#Cc1c(-c2ccccc2)nc(-c2cccc3oc4cc(-n5c6ccccc6c6ccc(-c7ccccc7)cc65)ccc4c23)nc1-c1ccccc1 ; N#Cc1c(-c2ccccc2)nc(-c2cccc3oc4ccc(-c5cccc(-n6c7ccccc7c7ccccc76)c5)cc4c23)nc1-c1ccccc1 ; N#Cc1c(-c2ccccc2)nc(-c2ccccc2)nc1-c1cccc2oc3cc(-n4c5ccccc5c5ccc(-c6ccccc6)cc54)ccc3c12 ; CC1(C)c2ccccc2-c2cc3c4ccccc4n(-c4ccc5c(c4)oc4cccc(-c6nc(-c7ccccc7)c(C#N)c(-c7ccccc7)n6)c45)c3cc21]

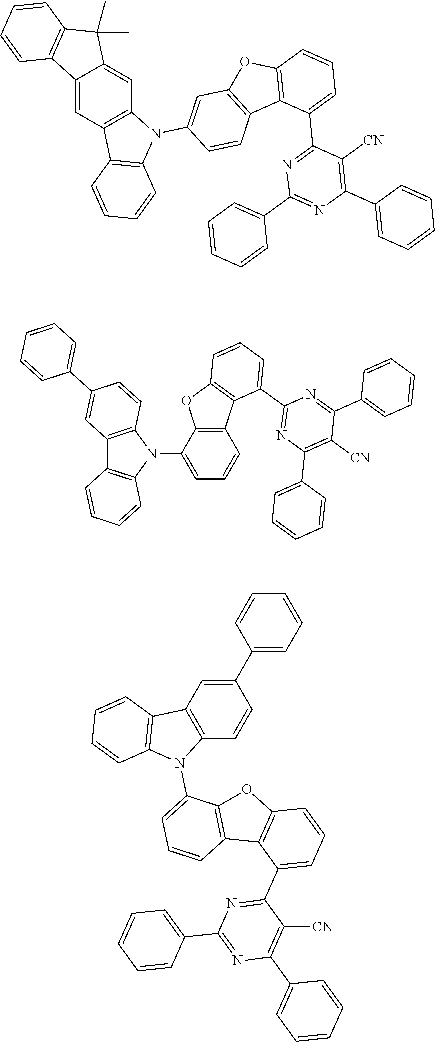 [SMILES: CC1(C)c2ccccc2-c2cc3c4ccccc4n(-c4ccc5c(c4)oc4cccc(-c6nc(-c7ccccc7)nc(-c7ccccc7)c6C#N)c45)c3cc21 ; N#Cc1c(-c2ccccc2)nc(-c2cccc3oc4c(-n5c6ccccc6c6cc(-c7ccccc7)ccc65)cccc4c23)nc1-c1ccccc1 ; N#Cc1c(-c2ccccc2)nc(-c2ccccc2)nc1-c1cccc2oc3c(-n4c5ccccc5c5cc(-c6ccccc6)ccc54)cccc3c12]

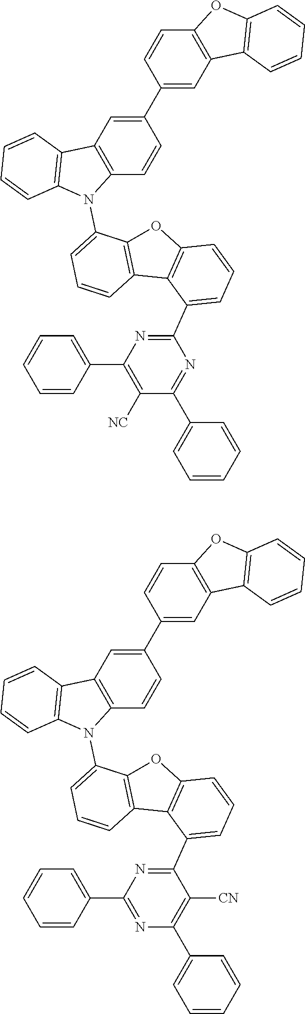 [SMILES: N#Cc1c(-c2ccccc2)nc(-c2cccc3oc4c(-n5c6ccccc6c6cc(-c7ccc8oc9ccccc9c8c7)ccc65)cccc4c23)nc1-c1ccccc1 ; N#Cc1c(-c2ccccc2)nc(-c2ccccc2)nc1-c1cccc2oc3c(-n4c5ccccc5c5cc(-c6ccc7oc8ccccc8c7c6)ccc54)cccc3c12]

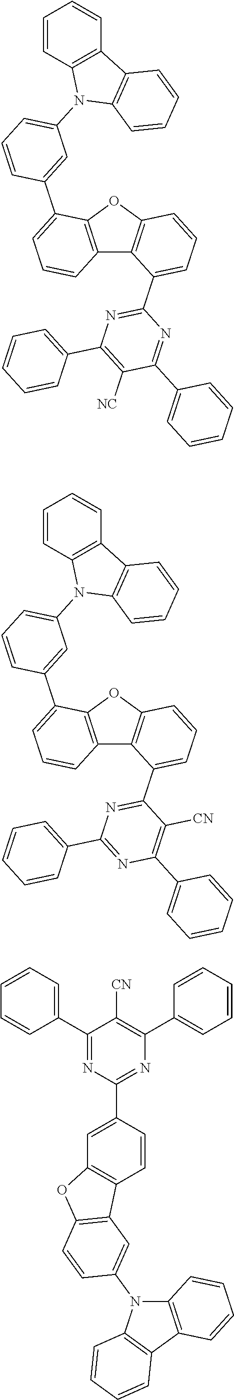

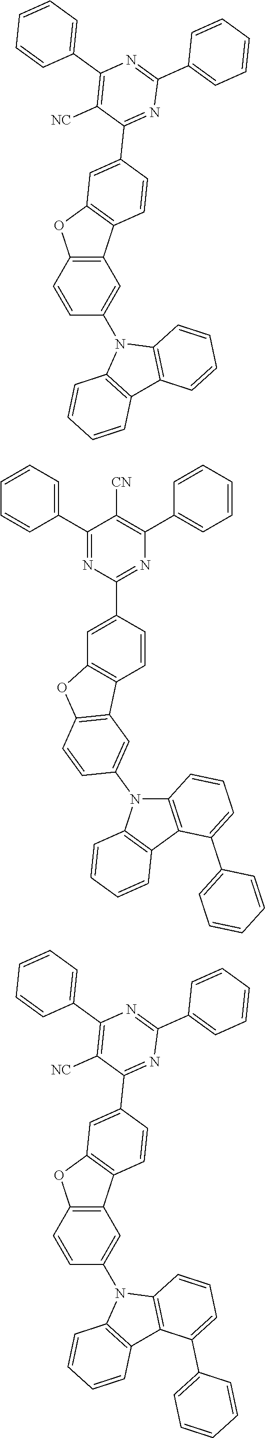

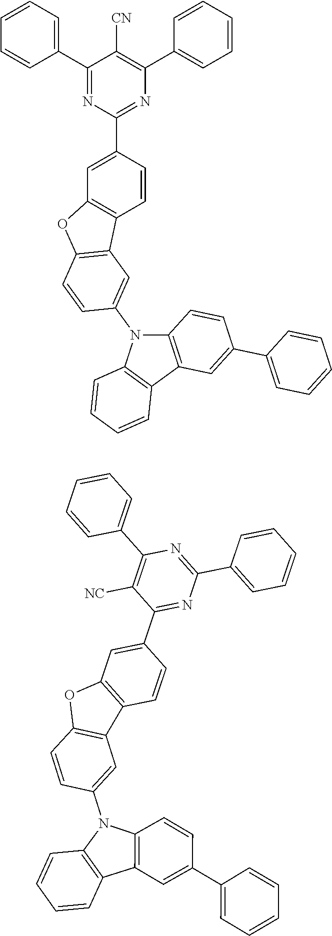

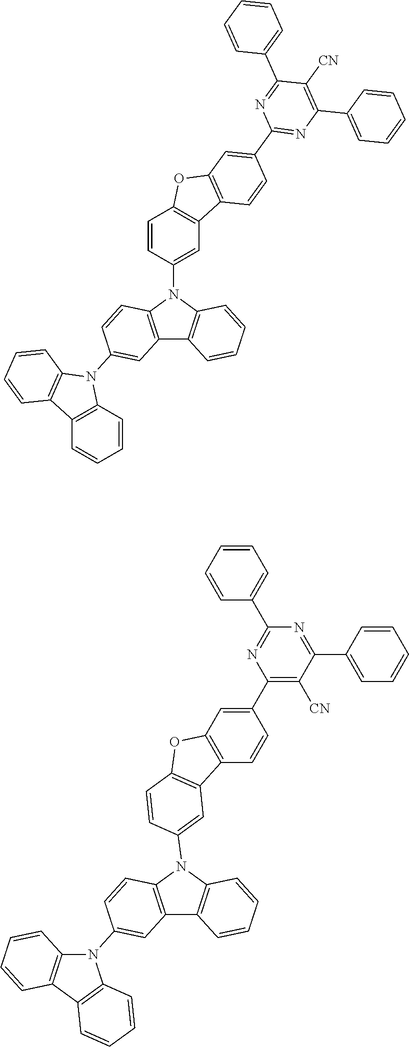 [SMILES: N#Cc1c(-c2ccccc2)nc(-c2ccc3c(c2)oc2ccc(-n4c5ccccc5c5cc(-n6c7ccccc7c7ccccc76)ccc54)cc23)nc1-c1ccccc1 ; N#Cc1c(-c2ccccc2)nc(-c2ccccc2)nc1-c1ccc2c(c1)oc1ccc(-n3c4ccccc4c4cc(-n5c6ccccc6c6ccccc65)ccc43)cc12]

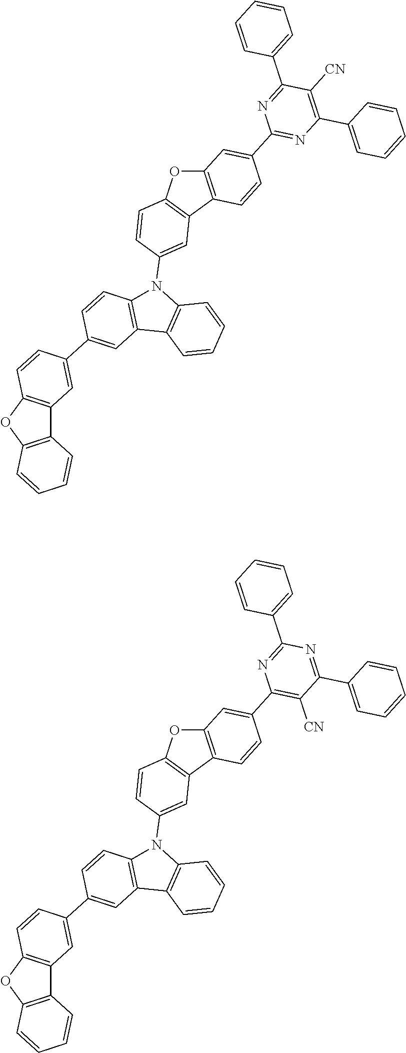 [SMILES: N#Cc1c(-c2ccccc2)nc(-c2ccc3c(c2)oc2ccc(-n4c5ccccc5c5cc(-c6ccc7oc8ccccc8c7c6)ccc54)cc23)nc1-c1ccccc1 ; N#Cc1c(-c2ccccc2)nc(-c2ccccc2)nc1-c1ccc2c(c1)oc1ccc(-n3c4ccccc4c4cc(-c5ccc6oc7ccccc7c6c5)ccc43)cc12]

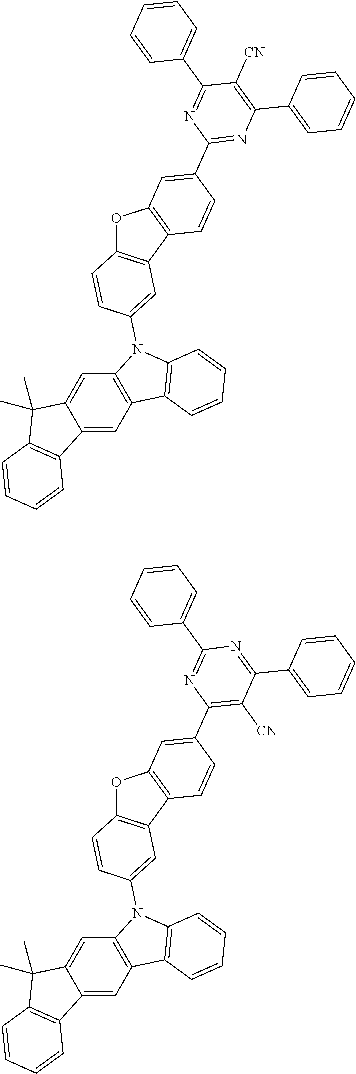 [SMILES: CC1(C)c2ccccc2-c2cc3c4ccccc4n(-c4ccc5oc6cc(-c7nc(-c8ccccc8)c(C#N)c(-c8ccccc8)n7)ccc6c5c4)c3cc21 ; CC1(C)c2ccccc2-c2cc3c4ccccc4n(-c4ccc5oc6cc(-c7nc(-c8ccccc8)nc(-c8ccccc8)c7C#N)ccc6c5c4)c3cc21]

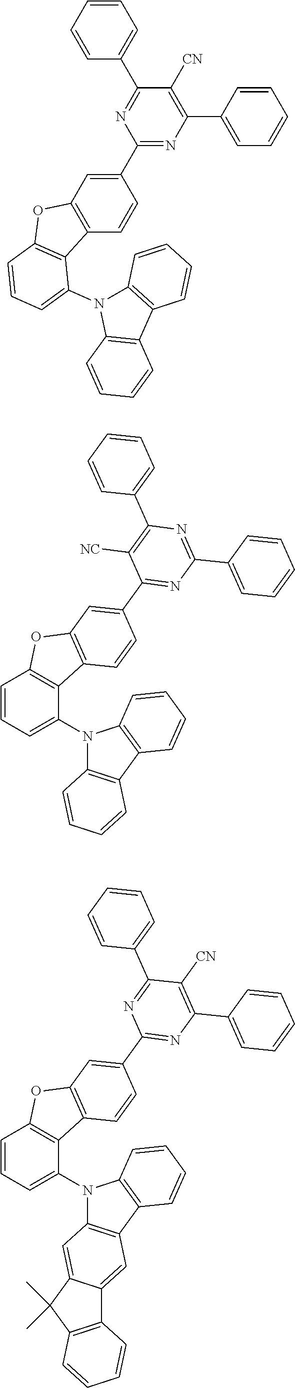

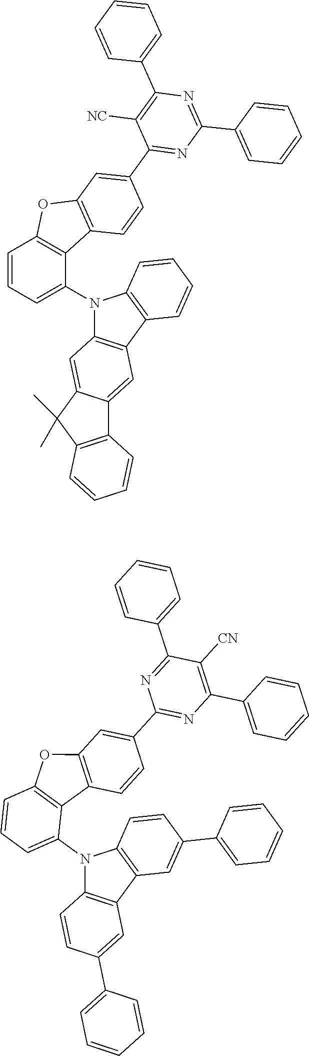 [SMILES: CC1(C)c2ccccc2-c2cc3c4ccccc4n(-c4cccc5oc6cc(-c7nc(-c8ccccc8)nc(-c8ccccc8)c7C#N)ccc6c45)c3cc21 ; N#Cc1c(-c2ccccc2)nc(-c2ccc3c(c2)oc2cccc(-n4c5ccc(-c6ccccc6)cc5c5cc(-c6ccccc6)ccc54)c23)nc1-c1ccccc1]

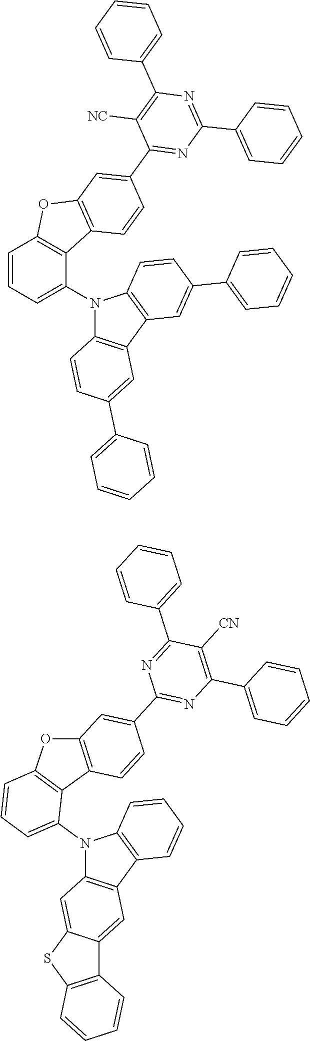

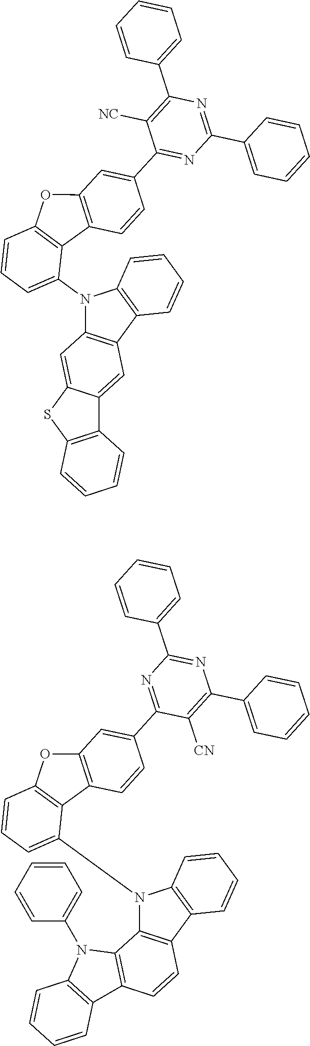 [SMILES: N#Cc1c(-c2ccccc2)nc(-c2ccccc2)nc1-c1ccc2c(c1)oc1cccc(-n3c4ccccc4c4cc5c(cc43)sc3ccccc35)c12 ; N#Cc1c(-c2ccccc2)nc(-c2ccccc2)nc1-c1ccc2c(c1)oc1cccc(-n3c4ccccc4c4ccc5c6ccccc6n(-c6ccccc6)c5c43)c12]

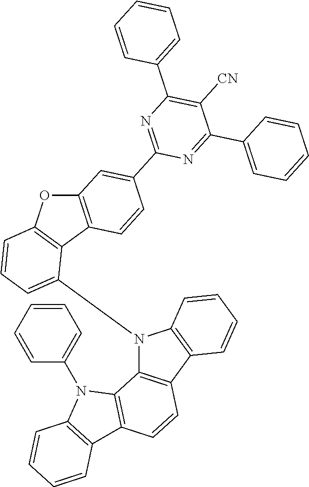 [SMILES: N#Cc1c(-c2ccccc2)nc(-c2ccc3c(c2)oc2cccc(-n4c5ccccc5c5ccc6c7ccccc7n(-c7ccccc7)c6c54)c23)nc1-c1ccccc1]

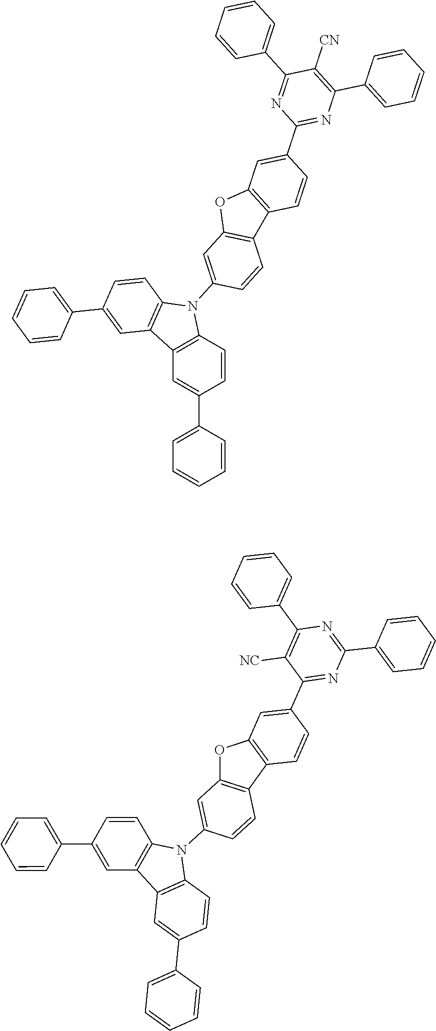 [SMILES: N#Cc1c(-c2ccccc2)nc(-c2ccc3c(c2)oc2cc(-n4c5ccc(-c6ccccc6)cc5c5cc(-c6ccccc6)ccc54)ccc23)nc1-c1ccccc1 ; N#Cc1c(-c2ccccc2)nc(-c2ccccc2)nc1-c1ccc2c(c1)oc1cc(-n3c4ccc(-c5ccccc5)cc4c4cc(-c5ccccc5)ccc43)ccc12]

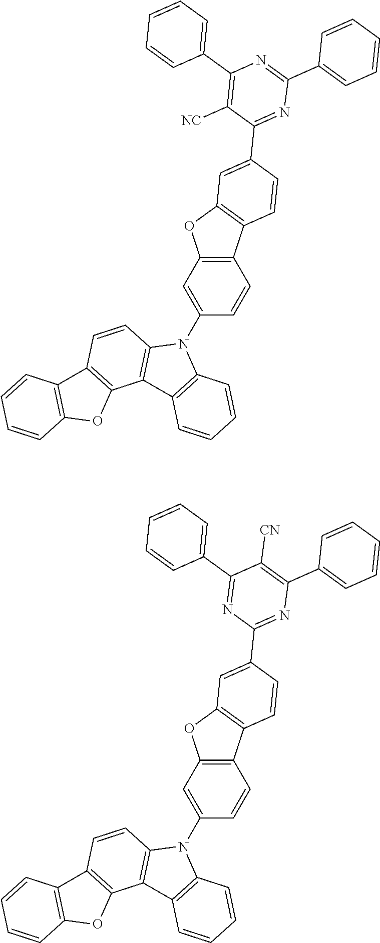 [SMILES: N#Cc1c(-c2ccccc2)nc(-c2ccccc2)nc1-c1ccc2c(c1)oc1cc(-n3c4ccccc4c4c5oc6ccccc6c5ccc43)ccc12 ; N#Cc1c(-c2ccccc2)nc(-c2ccc3c(c2)oc2cc(-n4c5ccccc5c5c6oc7ccccc7c6ccc54)ccc23)nc1-c1ccccc1]

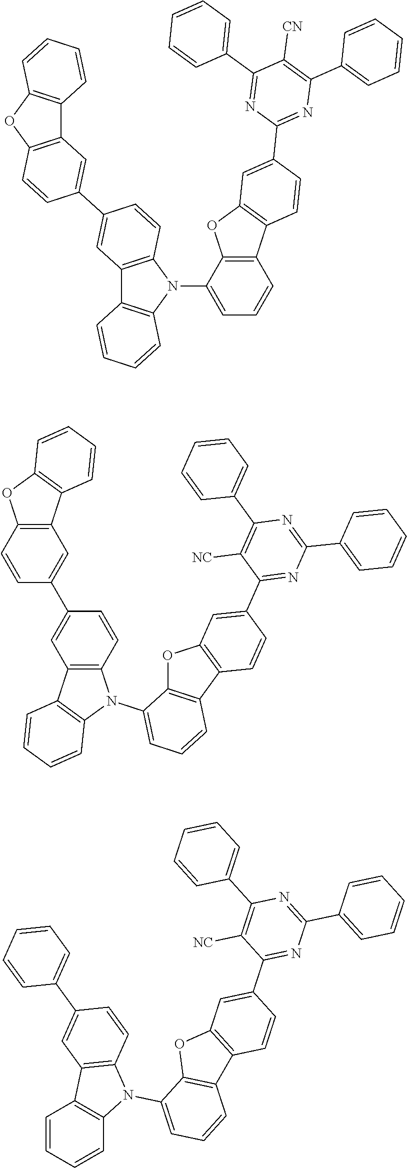 [SMILES: N#Cc1c(-c2ccccc2)nc(-c2ccc3c(c2)oc2c(-n4c5ccccc5c5cc(-c6ccc7oc8ccccc8c7c6)ccc54)cccc23)nc1-c1ccccc1 ; N#Cc1c(-c2ccccc2)nc(-c2ccccc2)nc1-c1ccc2c(c1)oc1c(-n3c4ccccc4c4cc(-c5ccc6oc7ccccc7c6c5)ccc43)cccc12 ; N#Cc1c(-c2ccccc2)nc(-c2ccccc2)nc1-c1ccc2c(c1)oc1c(-n3c4ccccc4c4cc(-c5ccccc5)ccc43)cccc12]

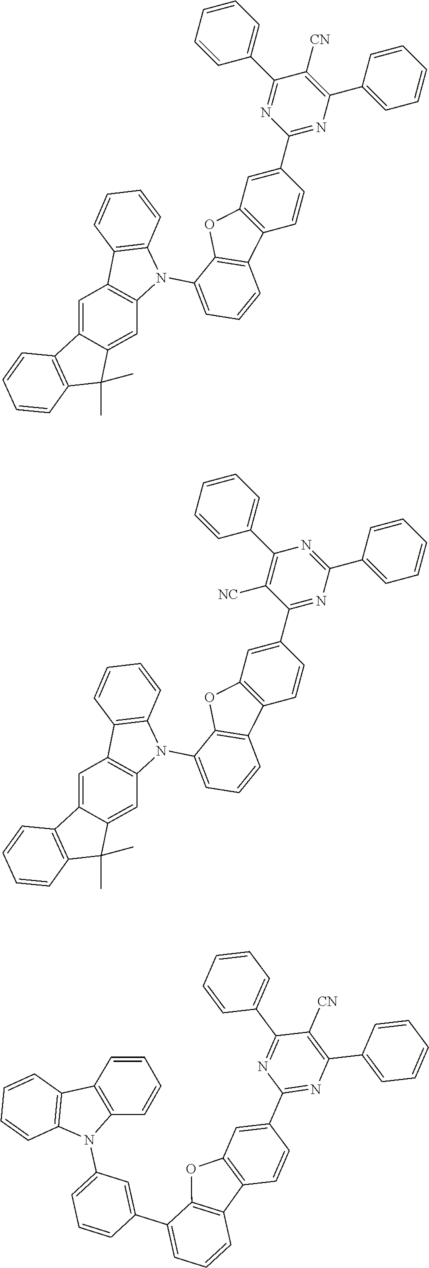 [SMILES: CC1(C)c2ccccc2-c2cc3c4ccccc4n(-c4cccc5c4oc4cc(-c6nc(-c7ccccc7)c(C#N)c(-c7ccccc7)n6)ccc45)c3cc21 ; CC1(C)c2ccccc2-c2cc3c4ccccc4n(-c4cccc5c4oc4cc(-c6nc(-c7ccccc7)nc(-c7ccccc7)c6C#N)ccc45)c3cc21 ; N#Cc1c(-c2ccccc2)nc(-c2ccc3c(c2)oc2c(-c4cccc(-n5c6ccccc6c6ccccc65)c4)cccc23)nc1-c1ccccc1]

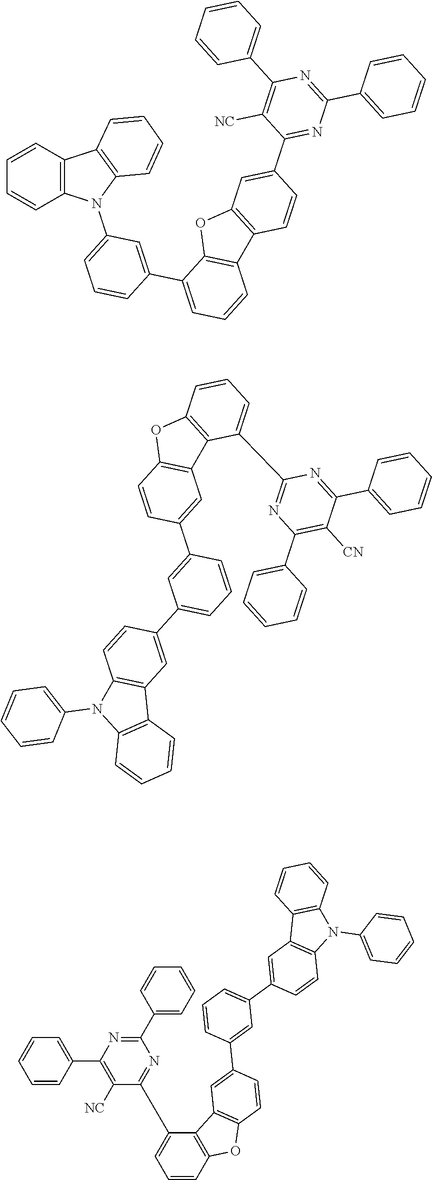 [SMILES: N#Cc1c(-c2ccccc2)nc(-c2ccccc2)nc1-c1ccc2c(c1)oc1c(-c3cccc(-n4c5ccccc5c5ccccc54)c3)cccc12 ; N#Cc1c(-c2ccccc2)nc(-c2cccc3oc4ccc(-c5cccc(-c6ccc7c(c6)c6ccccc6n7-c6ccccc6)c5)cc4c23)nc1-c1ccccc1 ; N#Cc1c(-c2ccccc2)nc(-c2ccccc2)nc1-c1cccc2oc3ccc(-c4cccc(-c5ccc6c(c5)c5ccccc5n6-c5ccccc5)c4)cc3c12]

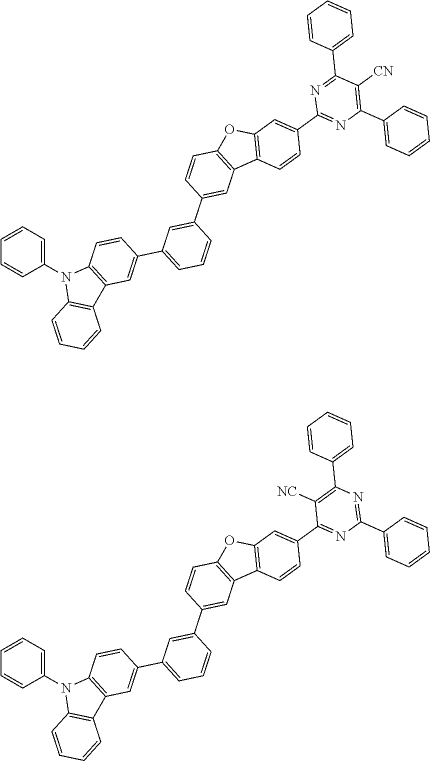 [SMILES: N#Cc1c(-c2ccccc2)nc(-c2ccc3c(c2)oc2ccc(-c4cccc(-c5ccc6c(c5)c5ccccc5n6-c5ccccc5)c4)cc23)nc1-c1ccccc1 ; N#Cc1c(-c2ccccc2)nc(-c2ccccc2)nc1-c1ccc2c(c1)oc1ccc(-c3cccc(-c4ccc5c(c4)c4ccccc4n5-c4ccccc4)c3)cc12]

In another aspect, the present disclosure also provides a polymer comprising at least one repeating unit, where at least one of the repeating unit comprises a structure of formula (I). In some embodiments, the polymer is a non-conjugated polymer in which the structural unit of formula (I) is on a side chain. In some embodiments, the polymer is conjugated polymer.

The term “small molecule” herein refers to a molecule that is no one of following: a polymer, an oligomer, a dendrimer, or a blend. In particular, there are no repeating structures in the small molecule. The molecular weight of the small molecule ≤3000 g/mol, preferably ≤2000 g/mol, and most preferably ≤1500 g/mol.

The term “polymer” comprises homopolymer, copolymer, and block copolymer. Also in the present disclosure, the term “polymer” comprises dendrimer. For the synthesis and application of the dendrimers please refer to [Dendrimers and Dendrons, Wiley-VCH Verlag GmbH & Co. KGaA, 2002, Ed. George R. Newkome, Charles N. Moorefield, Fritz Vogtle.].

In some embodiments, the synthetic method of the polymer is selected from SUZUKI-, YAMAMOTO-, STILLE-, NIGESHI-, KUMADA-, HECK-, SONOGASHIRA-, HIYAMA-, FUKUYAMA-, HARTWIG-BUCHWALD-, or ULLMAN-.

In some embodiments, the glass transition temperature (Tg) of the polymer ≥100° C., preferably ≥120° C., more preferably ≥140° C., further preferably ≥160° C., and most preferably ≥180° C.

In some embodiments, the polydispersity index (PDI) of the polymer is preferably from 1 to 5, more preferably 1 to 4, even more preferably 1 to 3, further preferably 1 to 2, and most preferably 1 to 1.5.

In some embodiments, the weight-average molecular weight (M_{w}) of the polymer is preferably from 10 k to 1 million, more preferably 50 k to 500 k, even more preferably 100 k to 400 k, further preferably 150 k to 300 k, and most preferably 200 k to 250 k.

In yet another aspect, the present disclosure further provides a mixture comprising an organic compound or a polymer as described herein, and at least one other organic functional material, the at least one other organic functional material may be selected from the group consisting of a hole-injection material (HIM), a hole-transport material (HTM), an electron-transport material (ETM), an electron-injection material (EIM), an electron-blocking material (EBM), a hole-blocking material (HBM), an emitting material, a host material (Host), and an organic dye. These organic functional materials are described in detail, for example, in WO2010135519A1, US20090134784A1, and WO2011110277A1. The entire contents of these three documents are incorporated herein for reference. The at least one other organic functional material can be a small molecule and polymer material.

In some embodiments, the mixture comprises an organic compound or a polymer as described herein, and a phosphorescent emitter. The organic compound of the present disclosure may be used here as a host with the weight percentage ≤20 wt %, preferably ≤15 wt %, more preferably ≤10 wt %, and most preferably ≤8 wt %.

In some embodiments, the mixture comprises an organic compound or a polymer as described herein, another host material, and a phosphorescent emitter. The organic compound of the present disclosure is used as a co-host material with the weight percentage ≥10 wt %, preferably ≥20 wt %, more preferably ≥30 wt %, and most preferably ≥40 wt %.

In some embodiments, the mixture comprises an organic compound or a polymer as described herein, a phosphorescent emitter, and a host material. In some embodiments, the organic compound as described herein can be used as an auxiliary emitting material, and the weight ratio of the organic compound and the phosphorescent emitter ranges from 1:2 to 2:1. In some embodiments, the T1 of the organic compound as described herein is higher than the phosphorescent emitter.

In some embodiments, the mixture comprises an organic compound or a polymer as described herein, and another TADF material.

In some embodiments, the mixture as described herein comprises a first organic compound (H1) and a second organic compound (H2), where the first organic compound (H1) comprises at least one of the organic compound or a polymer as described herein, and the second organic compound (H2) has hole-transporting property; preferably, the molar ratio of the first organic compound (H1) to the second organic compound (H2) ranges from 1:9 to 9:1.

In some embodiments, the second organic compound (H2) is selected from a hole-injection material (HIM), a hole-transport material (HTM), or an organic host material (Host).

In some embodiments, in the first organic compound (H1) and the second organic compound (H2) of the mixture as described herein, at least one of them has ((LUMO+1)−LUMO) ≥0.2 eV, preferably ≥0.25 eV, more preferably ≥0.3 eV, further preferably ≥0.35 eV, particularly preferably ≥0.4 eV, and most preferably ≥0.45 eV.

In some embodiments, in the mixture as described herein, the ((LUMO+1)−LUMO) of the first organic compound (H1) ≥0.2 eV, preferably ≥0.25 eV, more preferably ≥0.3 eV, further preferably ≥0.35 eV, particularly preferably ≥0.4 eV, and most preferably ≥0.45 eV.

In some embodiments, in the first organic compound (H1) and the second organic compound (H2) of the mixture as described herein, at least one of them has (HOMO−(HOMO−1)) ≥0.2 eV, preferably ≥0.25 eV, more preferably ≥0.3 eV, further preferably ≥0.35 eV, particularly preferably ≥0.4 eV, and most preferably ≥0.45 eV.

In some embodiments, in the mixture as described herein, the (HOMO−(HOMO−1)) of the second organic compound (H2) ≥0.2 eV, preferably ≥0.25 eV, more preferably ≥0.3 eV, further preferably ≥0.35 eV, particularly preferably ≥0.4 eV, and most preferably ≥0.45 eV.

In some embodiments, in the mixture as described herein, 1) the (S1−T1) of the first organic compound (H1) ≤0.30 eV, preferably ≤0.25 eV, more preferably ≤0.20 eV, and most preferably ≤0.10 eV, and/or 2) the LUMO of the second organic compound (H2) is higher than the LUMO of the first organic compound (H1), and the HOMO of the second organic compound (H2) is lower than the HOMO of the first organic compound (H1).

In some embodiments, in the mixture as described herein, the first organic compound (H1) and the second organic compound (H2) can form type II heterojunction energy structure, and min(LUMO(H1)−HOMO(H2), LUMO(H2)−HOMO(H1))≤min(E_{T}(H1), E_{T}(H2))+0.1 eV, where LUMO(H1), HOMO(H1), and E_{T}(H1) respectively represent the lowest unoccupied orbital level, the highest occupied orbital level, and the triplet energy level of the first organic compound (H1); LUMO(H2), HOMO(H2), and E_{T}(H2) respectively represent the lowest unoccupied orbital level, the highest occupied orbital level, and the triplet energy level of the second organic compound (H2). Preferably min(LUMO(H1)−HOMO(H2), LUMO(H2)−HOMO(H1))≤min(E_{T}(H1), E_{T}(H2)); more preferably min (LUMO(H1)−HOMO(H2), LUMO(H2)−HOMO(H1))≤min (E_{T}(H1), E_{T}(H2))−0.1 e V.

In some embodiments, the first organic compound (H1) and the second organic compound (H2) can form type I heterojunction energy structure, the singlet-triplet energy level difference (S1−T1) of the first organic compound (H1) or the second organic compound (H2) ≤0.25 eV, preferably ≤0.20 eV, more preferably ≤ 0.15 eV, and most preferably ≤0.10 eV.

In some embodiments, in the mixture as described herein, the molar ratio of the first organic compound (H1) to the second organic compound (H2) is from 1:9 to 9:1; preferably from 2:8 to 8:2; more preferably from 3:7 to 7:3; further preferably from 4:6 to 6:4; and most preferably from 4.5:5.5 to 5.5:4.5.

In some embodiments, in the mixture as described herein, the molecular weight difference between the first organic compound (H1) and the second organic compound (H2) ≤100 Dalton, preferably ≤80 Dalton, more preferably ≤70 Dalton, further preferably ≤60 Dalton, particularly preferably ≤40 Dalton, and most preferably ≤30 Dalton.

In some embodiments, in the mixture as described herein, the sublimation temperature difference between the first organic compound (H1) and the second organic compound (H2)≤50 K, preferably ≤30 K, more preferably ≤20 K, and most preferably ≤10 K.

In some embodiments, in the first organic compound (H1) and the second organic compound (H2) of the mixture as described herein, at least one of them has a glass transition temperature (Tg)≥100° C.; in some embodiments, at least one of them has a Tg≥120° C.; in some embodiments, at least one of them has a Tg≥140°° C.; in some embodiments, at least one of them has a Tg≥160° C.; in some embodiments, the Tg of at least one of them ≥180° C.

In some embodiments, the second organic compound (H2) of the mixture is a combination of formula (III-1), and formula (III-2) or formula (III-3):

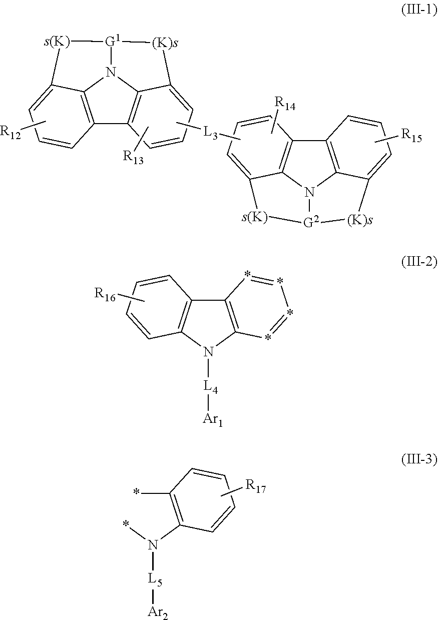

each of G₁ and G₂ at each occurrence is a substituted/unsubstituted aromatic or heteroaromatic group containing 5 to 30 ring atoms; K at each occurrence is a single bond or CR₁₈R₁₉; s at each occurrence is 0 or 1; the adjacent two *s in formula (III-2) are linked to formula (III-3), and the unlinked *s in both formula (III-2) and formula (III-3) are CR₂₀s; L₃-L₅ are identically defined as the above-mentioned L₁; R₁₂-R₁₇ are identically defined as the above-mentioned R₆; R₁₈-R₂₀ are identically defined as the above-mentioned R₁; each of Ar₁ and Ar₂ is a substituted/unsubstituted aromatic group containing 6 to 30 ring atom, or a substituted/unsubstituted heteroaromatic group containing 5 to 30 ring atom.

Further, each of Ar₁ and Ar₂ is independently selected from the following groups or any combination thereof:

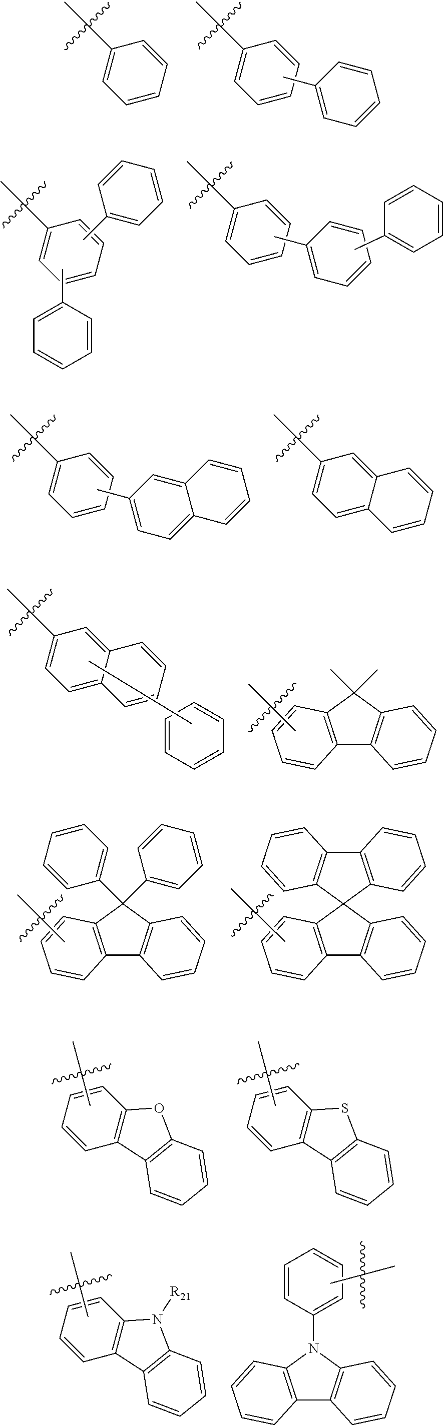

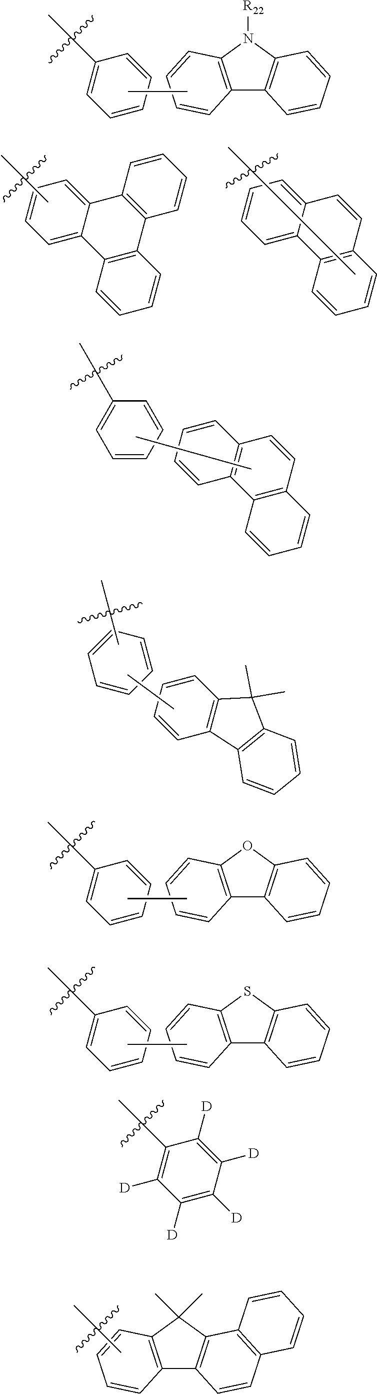

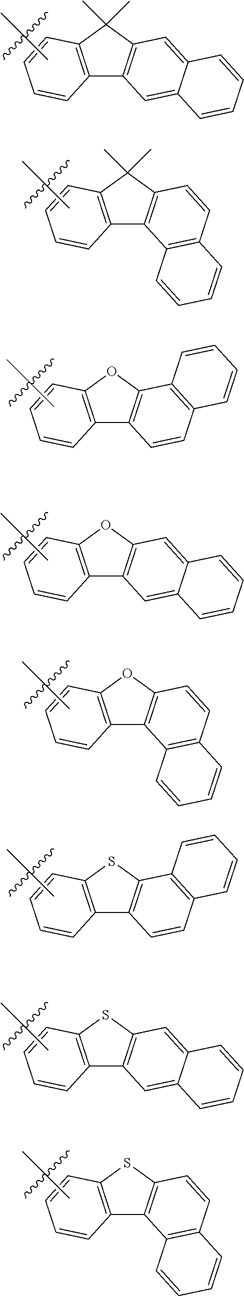

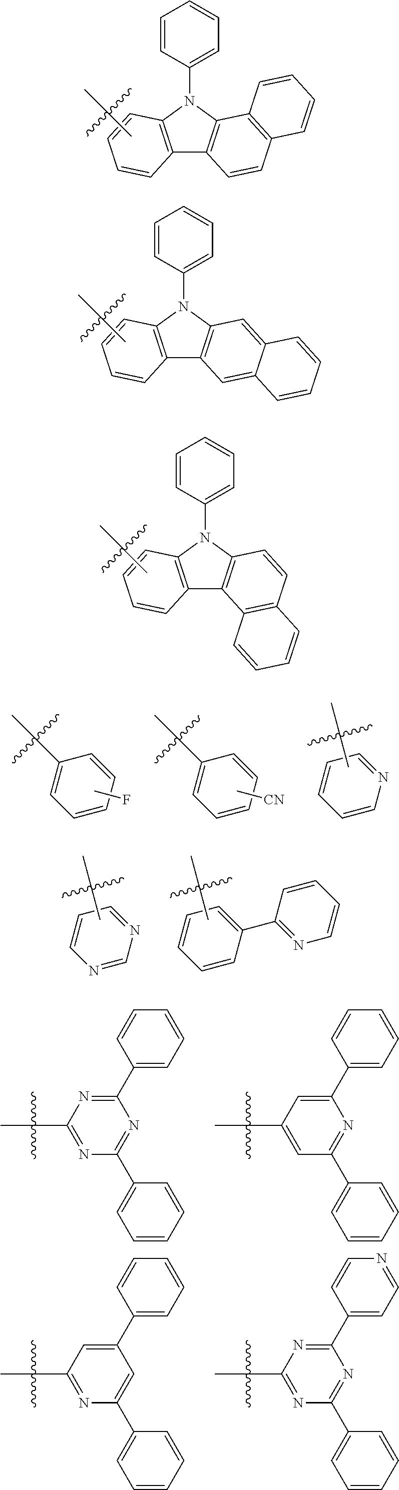

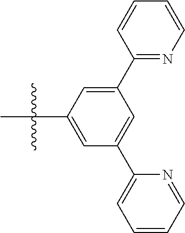 [SMILES: *c1cc(-c2ccccn2)cc(-c2ccccn2)c1]

R₂₁ and R₂₂ are identically defined as the above-mentioned R₁.

The above-mentioned groups are arbitrarily substituted with 0, 1, 2 or 3 groups that are selected from —D, —F, —Cl, —Br, a cyano group, a C₁-C₄ alkyl group, a C₁-C₃ haloalkyl group, a phenyl group, a naphthyl group, a fluorenyl group, a spirofluorenyl group, or a C₃-C₁₀ cycloalkyl group.

In some embodiments, the second organic compound (H2) of the mixture is a compound of formulas (IV-1)-(IV-6):

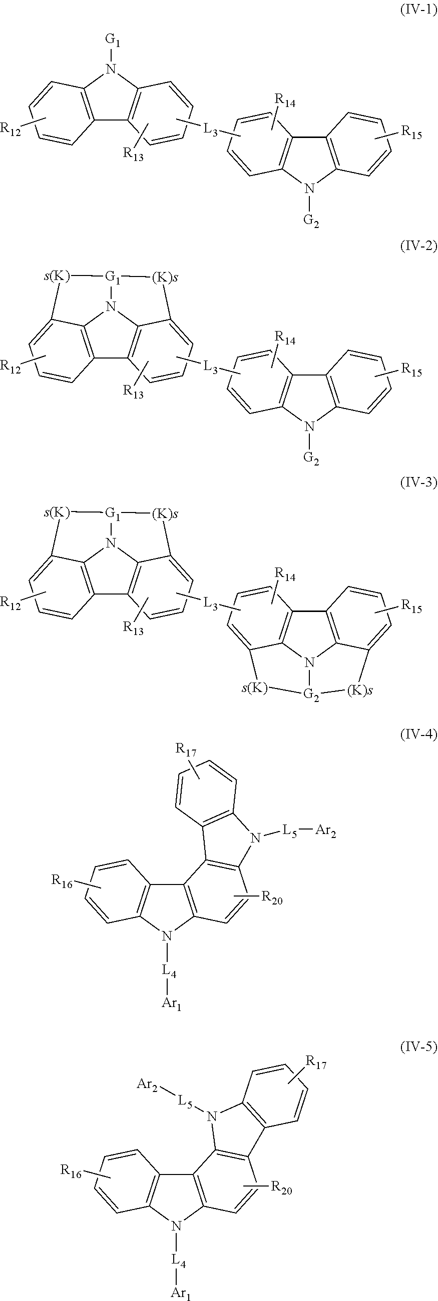

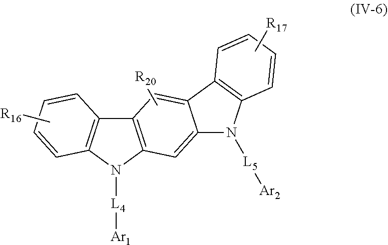

Where G₁, G₂, K, R₁₂-R₁₇, R₂₀, L₃-L₅, Ar₁, Ar₂, and s are identically defined as described herein.

Specific examples of the second organic compounds (H2) are listed below, but not limited thereto.

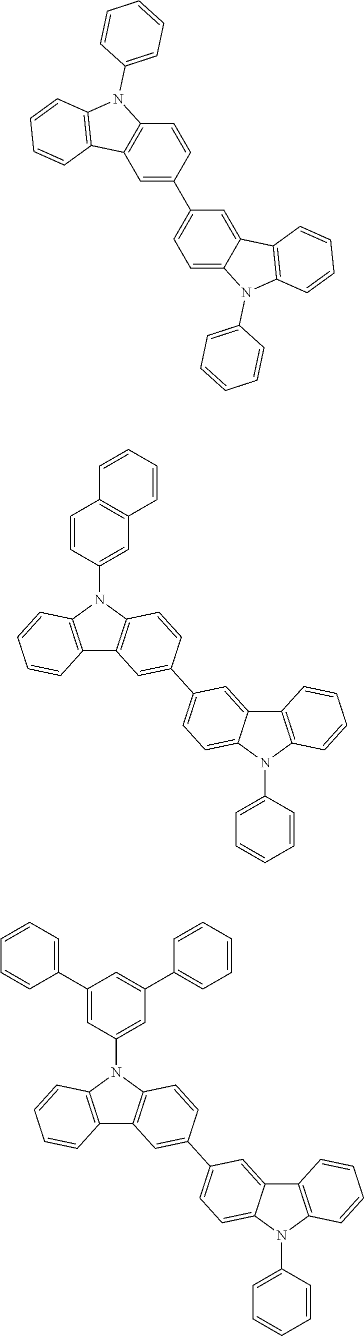 [SMILES: c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4ccc5ccccc5c4)ccc32)cc1 ; c1ccc(-c2cc(-c3ccccc3)cc(-n3c4ccccc4c4cc(-c5ccc6c(c5)c5ccccc5n6-c5ccccc5)ccc43)c2)cc1]

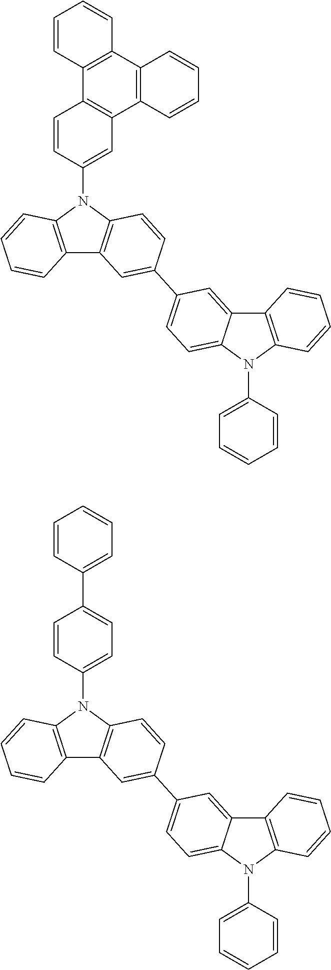 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-c2ccc(-n3c4ccccc4c4cc(-c5ccc6c(c5)c5ccccc5n6-c5ccccc5)ccc43)cc2)cc1]

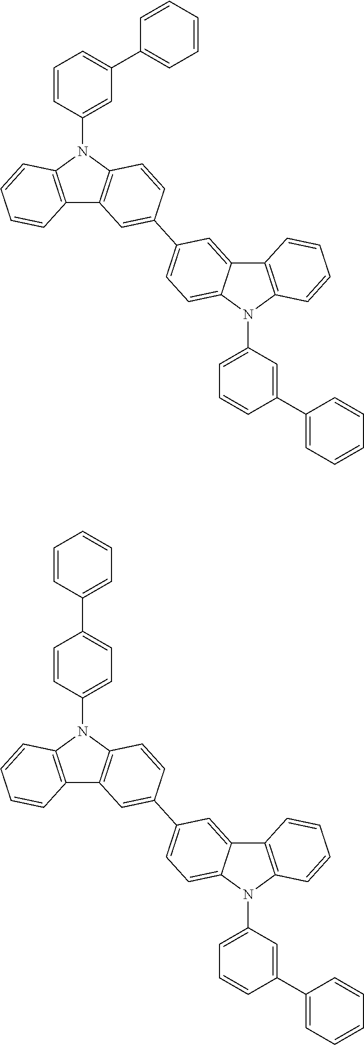 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

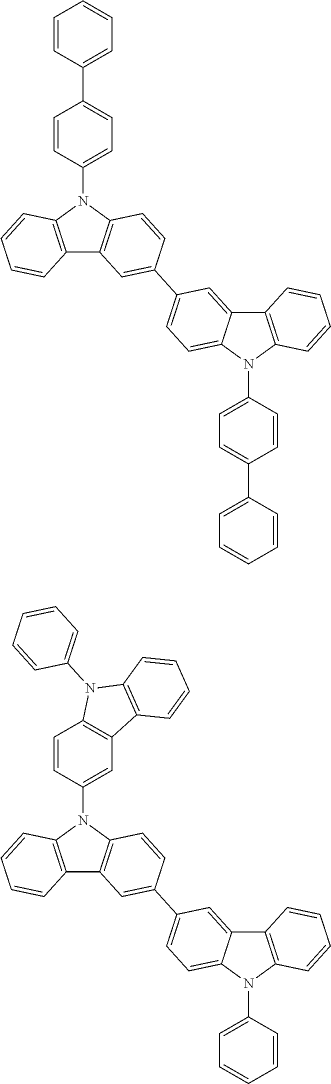 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-n2c3ccccc3c3cc(-c4ccc5c(c4)c4ccccc4n5-c4ccc5c(c4)c4ccccc4n5-c4ccccc4)ccc32)cc1]

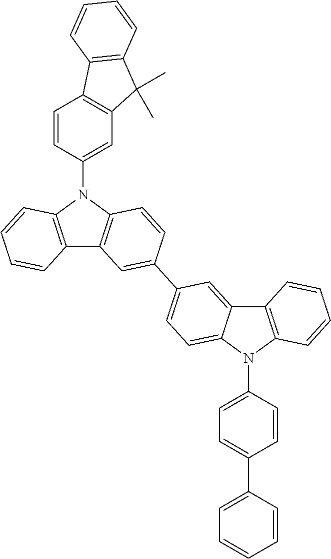 [SMILES: CC1(C)c2ccccc2-c2ccc(-n3c4ccccc4c4cc(-c5ccc6c(c5)c5ccccc5n6-c5ccc(-c6ccccc6)cc5)ccc43)cc21]

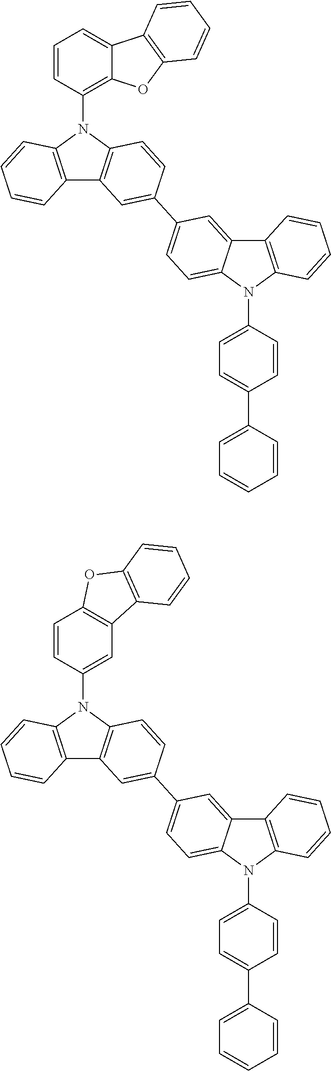 [SMILES: c1ccc(-c2ccc(-n3c4ccccc4c4cc(-c5ccc6c(c5)c5ccccc5n6-c5cccc6c5oc5ccccc56)ccc43)cc2)cc1 ; c1ccc(-c2ccc(-n3c4ccccc4c4cc(-c5ccc6c(c5)c5ccccc5n6-c5ccc6oc7ccccc7c6c5)ccc43)cc2)cc1]

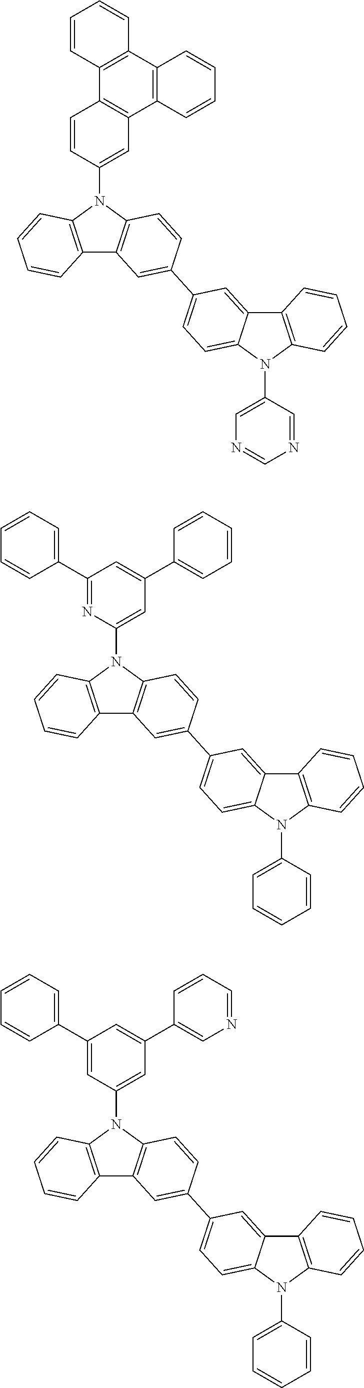 [SMILES: c1ccc2c(c1)c1ccccc1c1cc(-n3c4ccccc4c4cc(-c5ccc6c(c5)c5ccccc5n6-c5cncnc5)ccc43)ccc21 ; c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-c3cccnc3)cc(-n3c4ccccc4c4cc(-c5ccc6c(c5)c5ccccc5n6-c5ccccc5)ccc43)c2)cc1]

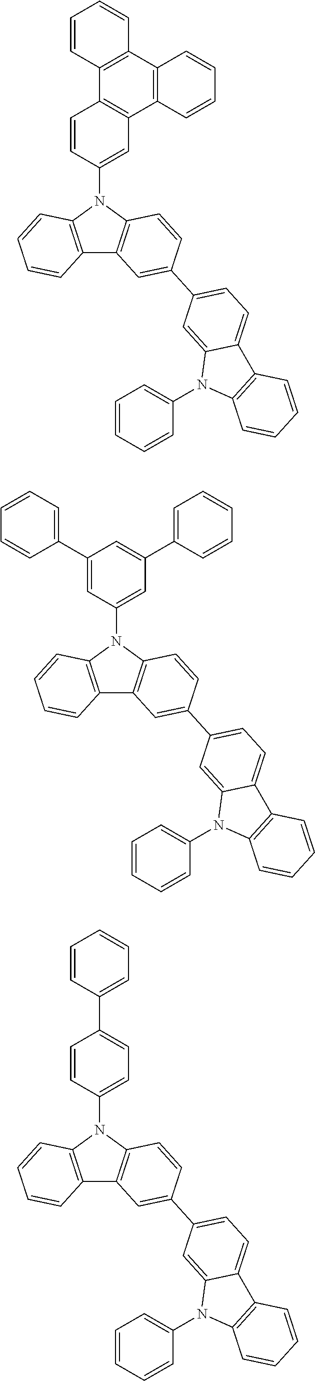 [SMILES: c1ccc(-n2c3ccccc3c3ccc(-c4ccc5c(c4)c4ccccc4n5-c4ccc5c6ccccc6c6ccccc6c5c4)cc32)cc1 ; c1ccc(-c2cc(-c3ccccc3)cc(-n3c4ccccc4c4cc(-c5ccc6c7ccccc7n(-c7ccccc7)c6c5)ccc43)c2)cc1 ; c1ccc(-c2ccc(-n3c4ccccc4c4cc(-c5ccc6c7ccccc7n(-c7ccccc7)c6c5)ccc43)cc2)cc1]

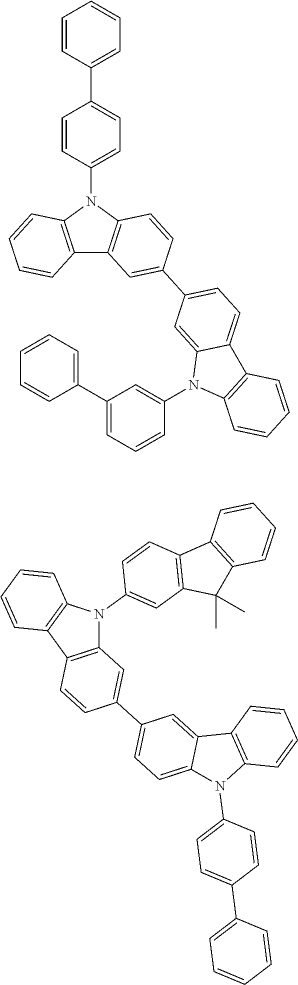 [SMILES: c1ccc(-c2ccc(-n3c4ccccc4c4cc(-c5ccc6c7ccccc7n(-c7cccc(-c8ccccc8)c7)c6c5)ccc43)cc2)cc1 ; CC1(C)c2ccccc2-c2ccc(-n3c4ccccc4c4ccc(-c5ccc6c(c5)c5ccccc5n6-c5ccc(-c6ccccc6)cc5)cc43)cc21]

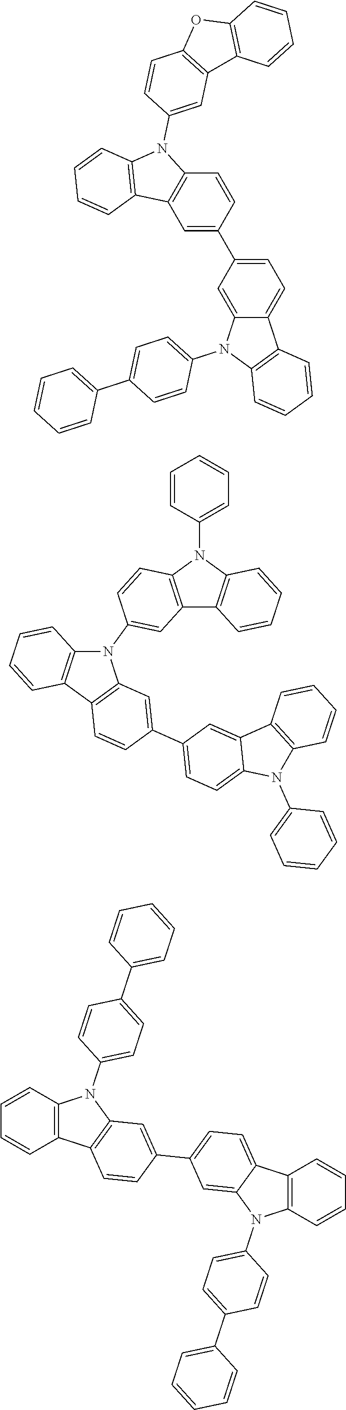 [SMILES: c1ccc(-c2ccc(-n3c4ccccc4c4ccc(-c5ccc6c(c5)c5ccccc5n6-c5ccc6oc7ccccc7c6c5)cc43)cc2)cc1 ; c1ccc(-n2c3ccccc3c3cc(-c4ccc5c6ccccc6n(-c6ccc7c(c6)c6ccccc6n7-c6ccccc6)c5c4)ccc32)cc1 ; c1ccc(-c2ccc(-n3c4ccccc4c4ccc(-c5ccc6c7ccccc7n(-c7ccc(-c8ccccc8)cc7)c6c5)cc43)cc2)cc1]

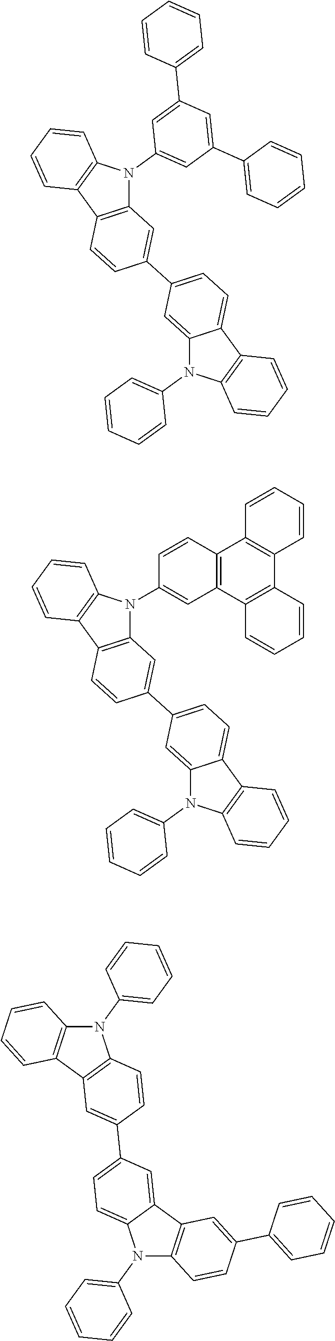 [SMILES: c1ccc(-c2cc(-c3ccccc3)cc(-n3c4ccccc4c4ccc(-c5ccc6c7ccccc7n(-c7ccccc7)c6c5)cc43)c2)cc1 ; c1ccc(-n2c3ccccc3c3ccc(-c4ccc5c6ccccc6n(-c6ccc7c8ccccc8c8ccccc8c7c6)c5c4)cc32)cc1 ; c1ccc(-c2ccc3c(c2)c2cc(-c4ccc5c(c4)c4ccccc4n5-c4ccccc4)ccc2n3-c2ccccc2)cc1]

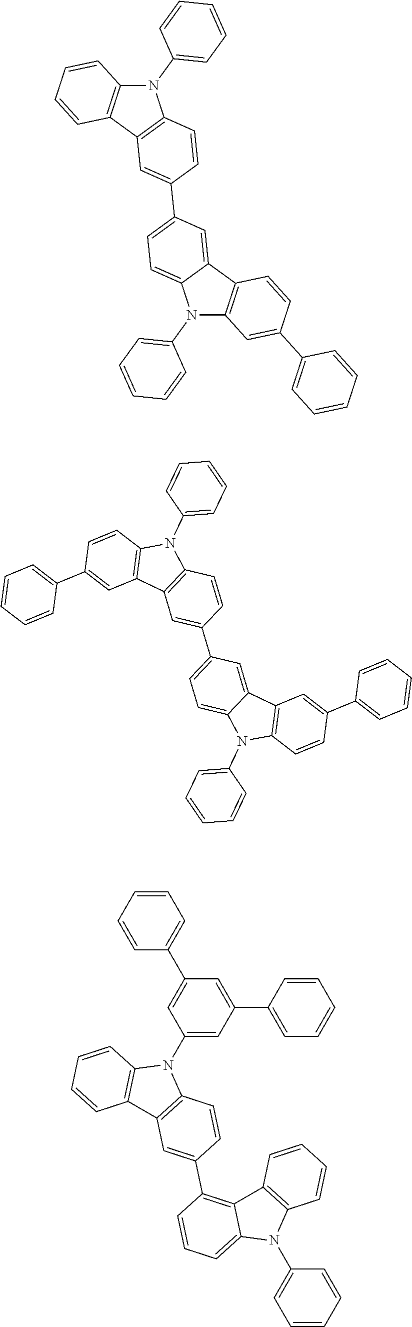 [SMILES: c1ccc(-c2ccc3c4cc(-c5ccc6c(c5)c5ccccc5n6-c5ccccc5)ccc4n(-c4ccccc4)c3c2)cc1 ; c1ccc(-c2ccc3c(c2)c2cc(-c4ccc5c(c4)c4cc(-c6ccccc6)ccc4n5-c4ccccc4)ccc2n3-c2ccccc2)cc1 ; c1ccc(-c2cc(-c3ccccc3)cc(-n3c4ccccc4c4cc(-c5cccc6c5c5ccccc5n6-c5ccccc5)ccc43)c2)cc1]

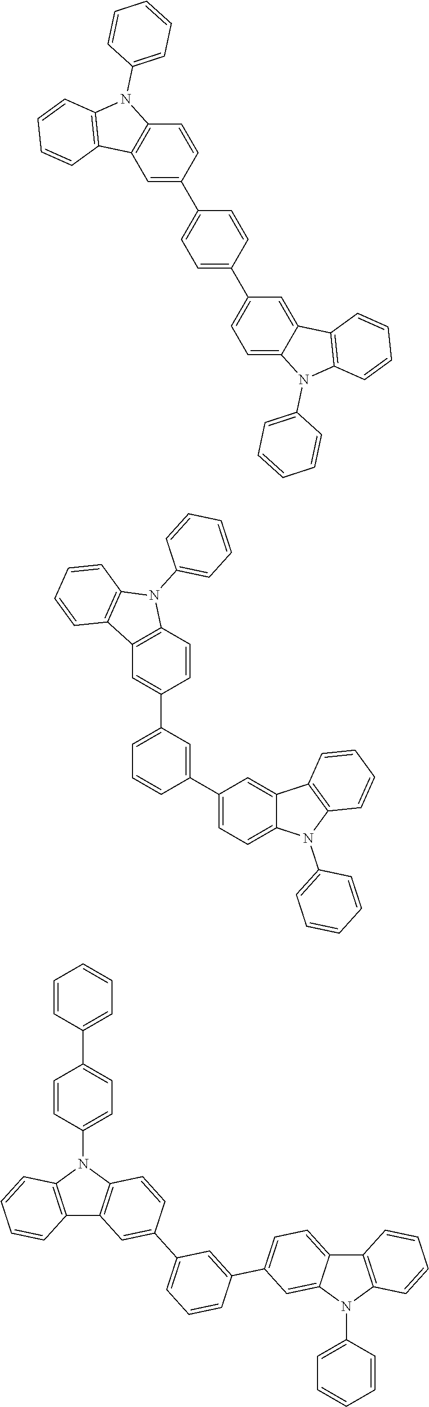 [SMILES: c1ccc(-n2c3ccccc3c3cc(-c4ccc(-c5ccc6c(c5)c5ccccc5n6-c5ccccc5)cc4)ccc32)cc1 ; c1ccc(-n2c3ccccc3c3cc(-c4cccc(-c5ccc6c(c5)c5ccccc5n6-c5ccccc5)c4)ccc32)cc1 ; c1ccc(-c2ccc(-n3c4ccccc4c4cc(-c5cccc(-c6ccc7c8ccccc8n(-c8ccccc8)c7c6)c5)ccc43)cc2)cc1]

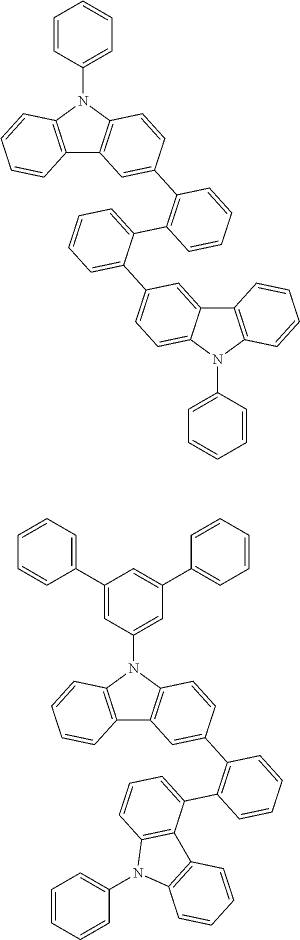 [SMILES: c1ccc(-n2c3ccccc3c3cc(-c4ccccc4-c4ccccc4-c4ccc5c(c4)c4ccccc4n5-c4ccccc4)ccc32)cc1 ; c1ccc(-c2cc(-c3ccccc3)cc(-n3c4ccccc4c4cc(-c5ccccc5-c5cccc6c5c5ccccc5n6-c5ccccc5)ccc43)c2)cc1]

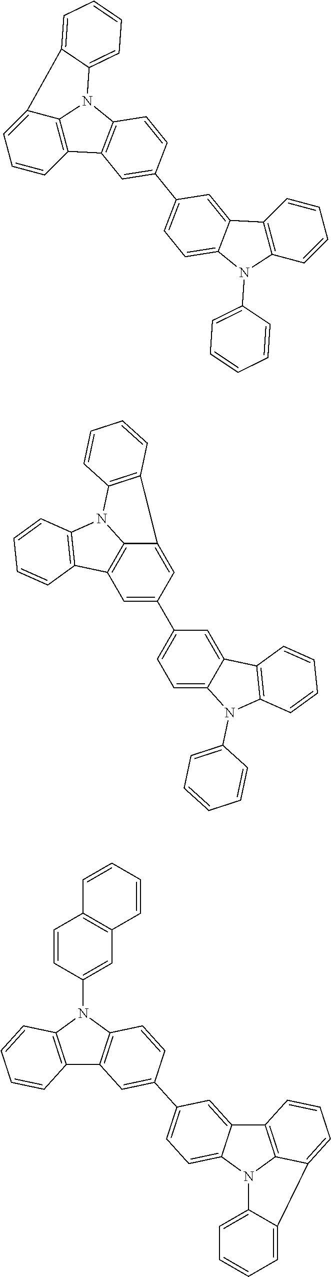 [SMILES: c1ccc(-n2c3ccccc3c3cc(-c4ccc5c(c4)c4cccc6c7ccccc7n5c64)ccc32)cc1 ; c1ccc(-n2c3ccccc3c3cc(-c4cc5c6ccccc6n6c7ccccc7c(c4)c56)ccc32)cc1 ; c1ccc2cc(-n3c4ccccc4c4cc(-c5ccc6c(c5)c5cccc7c8ccccc8n6c75)ccc43)ccc2c1]

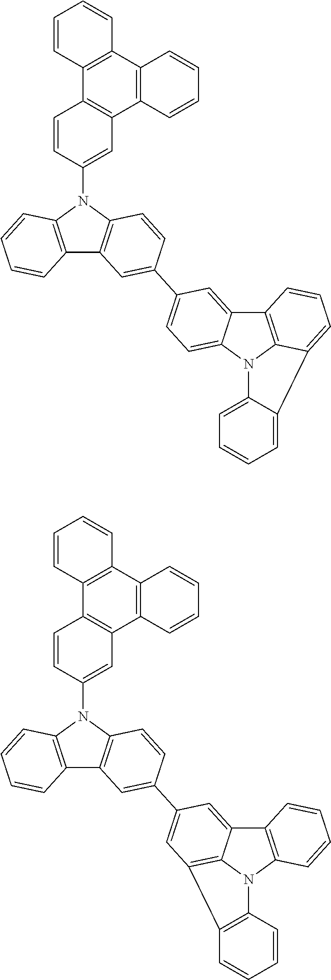 [SMILES: c1ccc2c(c1)c1ccccc1c1cc(-n3c4ccccc4c4cc(-c5ccc6c(c5)c5cccc7c8ccccc8n6c75)ccc43)ccc21 ; c1ccc2c(c1)c1ccccc1c1cc(-n3c4ccccc4c4cc(-c5cc6c7ccccc7n7c8ccccc8c(c5)c67)ccc43)ccc21]

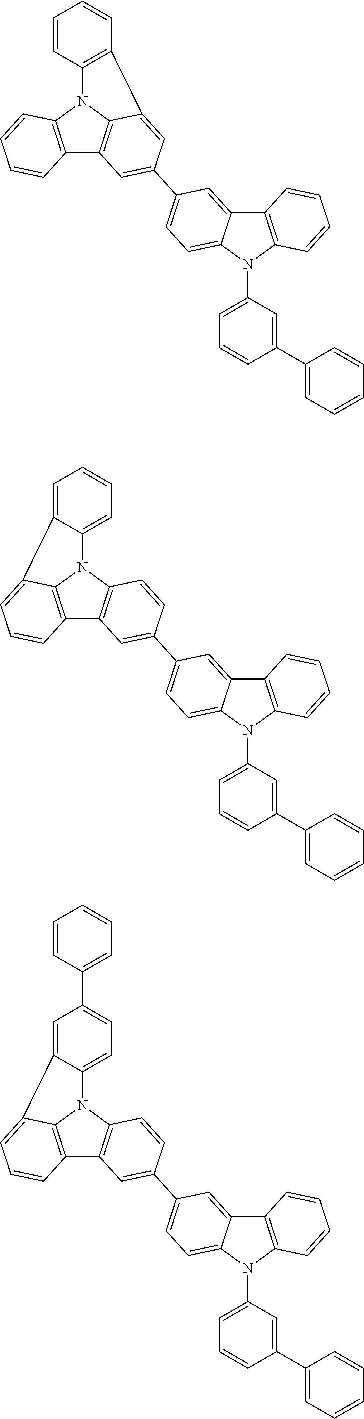 [SMILES: c1ccc(-c2cccc(-n3c4ccccc4c4cc(-c5cc6c7ccccc7n7c8ccccc8c(c5)c67)ccc43)c2)cc1 ; c1ccc(-c2cccc(-n3c4ccccc4c4cc(-c5ccc6c(c5)c5cccc7c8ccccc8n6c75)ccc43)c2)cc1 ; c1ccc(-c2cccc(-n3c4ccccc4c4cc(-c5ccc6c(c5)c5cccc7c8cc(-c9ccccc9)ccc8n6c75)ccc43)c2)cc1]

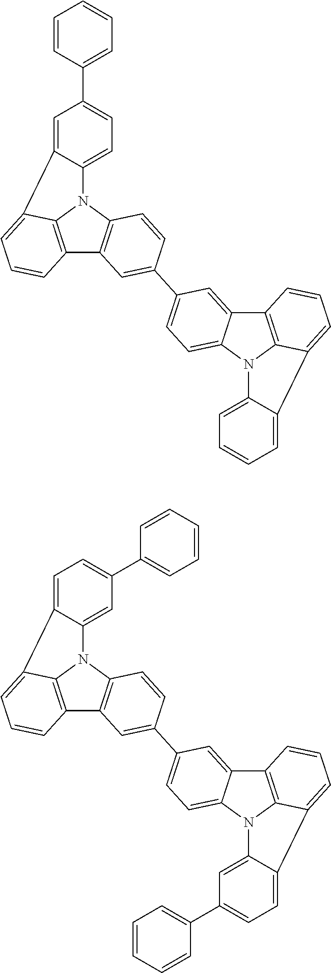 [SMILES: c1ccc(-c2ccc3c(c2)c2cccc4c5cc(-c6ccc7c(c6)c6cccc8c9ccccc9n7c86)ccc5n3c24)cc1 ; c1ccc(-c2ccc3c4cccc5c6cc(-c7ccc8c(c7)c7cccc9c%10ccc(-c%11ccccc%11)cc%10n8c79)ccc6n(c3c2)c54)cc1]

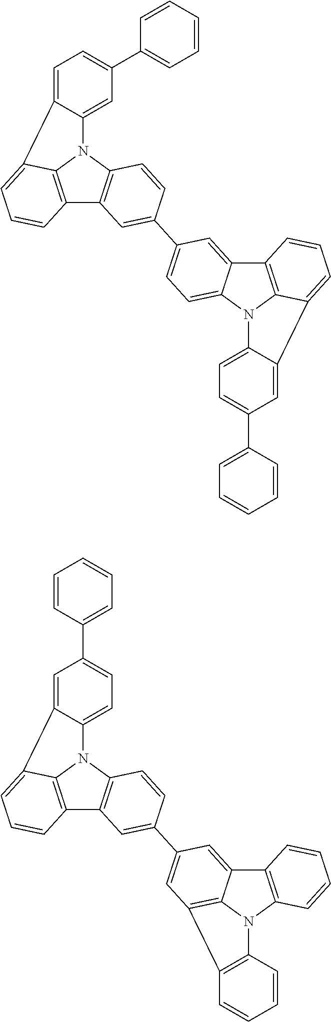 [SMILES: c1ccc(-c2ccc3c(c2)c2cccc4c5cc(-c6ccc7c(c6)c6cccc8c9ccc(-c%10ccccc%10)cc9n7c68)ccc5n3c24)cc1 ; c1ccc(-c2ccc3c(c2)c2cccc4c5cc(-c6cc7c8ccccc8n8c9ccccc9c(c6)c78)ccc5n3c24)cc1]

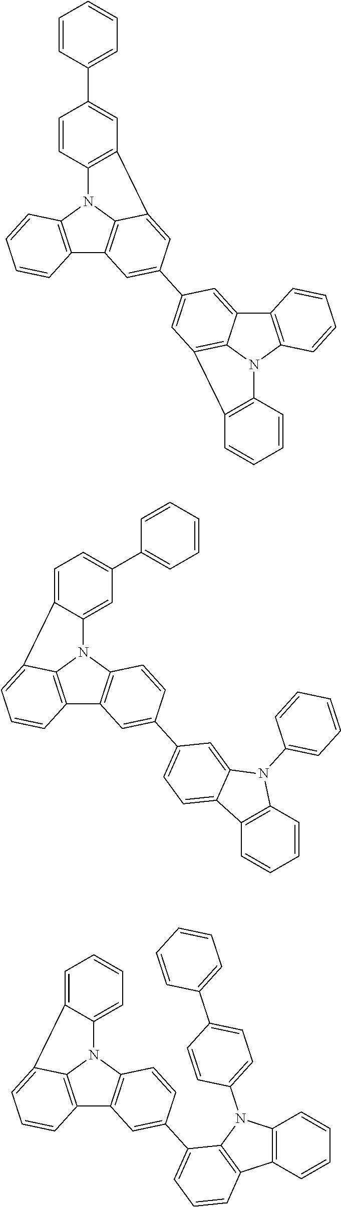 [SMILES: c1ccc(-c2ccc3c(c2)c2cc(-c4cc5c6ccccc6n6c7ccccc7c(c4)c56)cc4c5ccccc5n3c42)cc1 ; c1ccc(-c2ccc3c4cccc5c6cc(-c7ccc8c9ccccc9n(-c9ccccc9)c8c7)ccc6n(c3c2)c54)cc1 ; c1ccc(-c2ccc(-n3c4ccccc4c4cccc(-c5ccc6c(c5)c5cccc7c8ccccc8n6c75)c43)cc2)cc1]

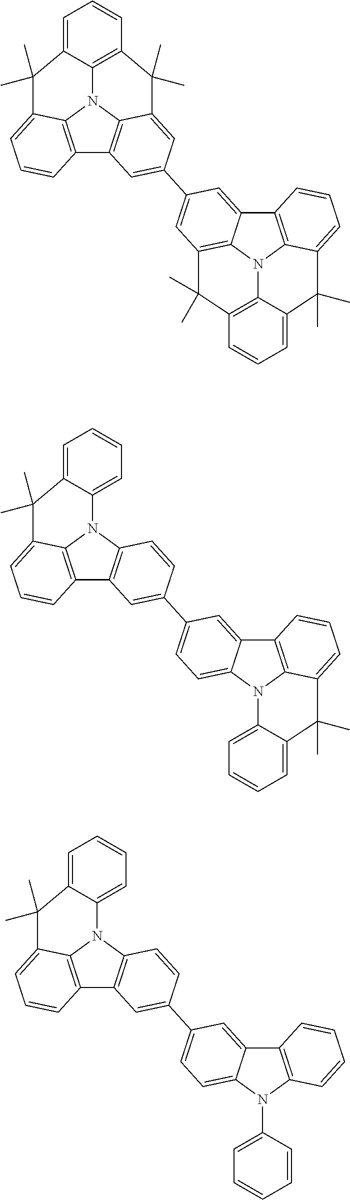

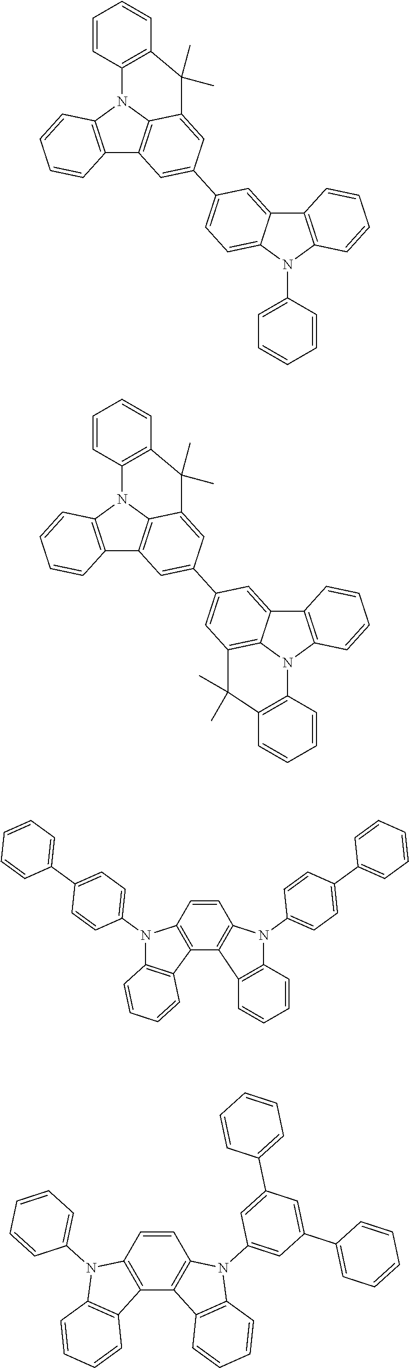 [SMILES: CC1(C)c2ccccc2-n2c3ccccc3c3cc(-c4ccc5c(c4)c4ccccc4n5-c4ccccc4)cc1c32 ; CC1(C)c2ccccc2-n2c3ccccc3c3cc(-c4cc5c6c(c4)c4ccccc4n6-c4ccccc4C5(C)C)cc1c32 ; c1ccc(-c2ccc(-n3c4ccccc4c4c5c6ccccc6n(-c6ccc(-c7ccccc7)cc6)c5ccc43)cc2)cc1 ; c1ccc(-c2cc(-c3ccccc3)cc(-n3c4ccccc4c4c5c6ccccc6n(-c6ccccc6)c5ccc43)c2)cc1]

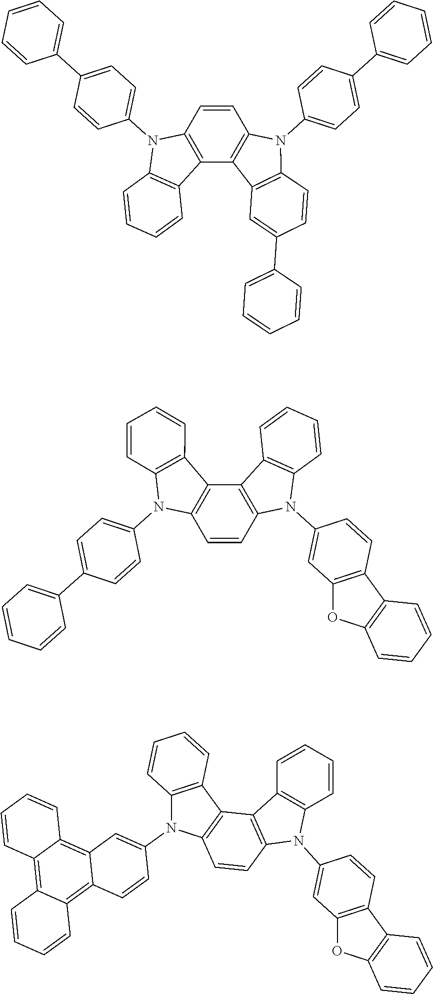 [SMILES: c1ccc(-c2ccc(-n3c4ccccc4c4c5c6cc(-c7ccccc7)ccc6n(-c6ccc(-c7ccccc7)cc6)c5ccc43)cc2)cc1 ; c1ccc(-c2ccc(-n3c4ccccc4c4c5c6ccccc6n(-c6ccc7c(c6)oc6ccccc67)c5ccc43)cc2)cc1 ; c1ccc2c(c1)oc1cc(-n3c4ccccc4c4c5c6ccccc6n(-c6ccc7c8ccccc8c8ccccc8c7c6)c5ccc43)ccc12]

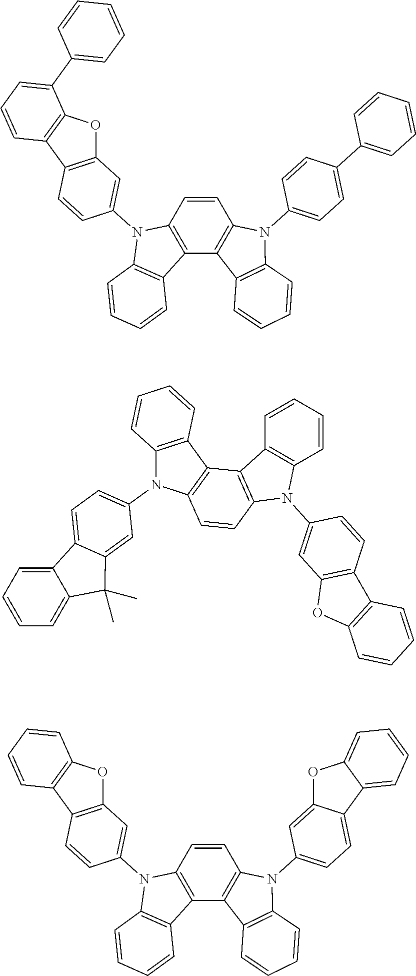 [SMILES: c1ccc(-c2ccc(-n3c4ccccc4c4c5c6ccccc6n(-c6ccc7c(c6)oc6c(-c8ccccc8)cccc67)c5ccc43)cc2)cc1 ; CC1(C)c2ccccc2-c2ccc(-n3c4ccccc4c4c5c6ccccc6n(-c6ccc7c(c6)oc6ccccc67)c5ccc43)cc21 ; c1ccc2c(c1)oc1cc(-n3c4ccccc4c4c5c6ccccc6n(-c6ccc7c(c6)oc6ccccc67)c5ccc43)ccc12]

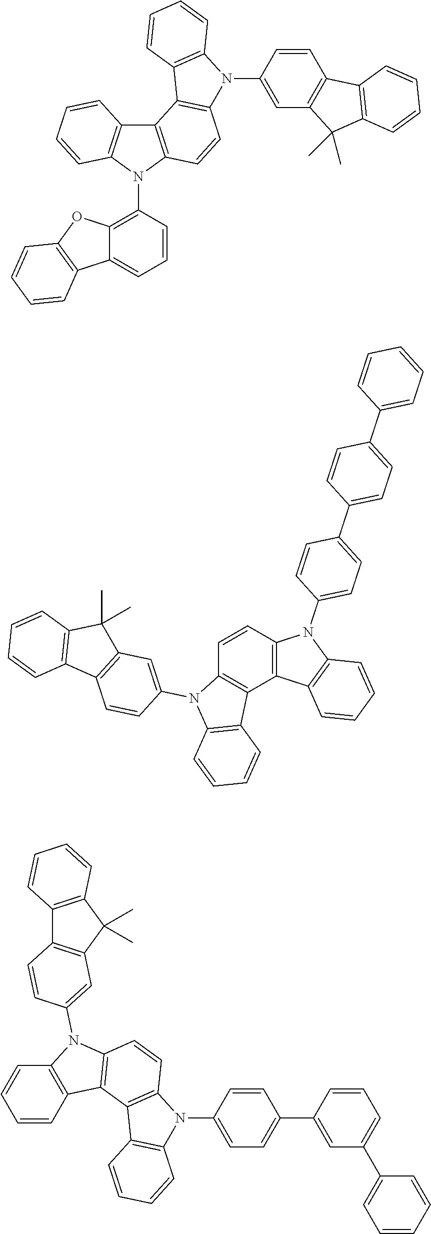 [SMILES: CC1(C)c2ccccc2-c2ccc(-n3c4ccccc4c4c5c6ccccc6n(-c6cccc7c6oc6ccccc67)c5ccc43)cc21 ; CC1(C)c2ccccc2-c2ccc(-n3c4ccccc4c4c5c6ccccc6n(-c6ccc(-c7ccc(-c8ccccc8)cc7)cc6)c5ccc43)cc21 ; CC1(C)c2ccccc2-c2ccc(-n3c4ccccc4c4c5c6ccccc6n(-c6ccc(-c7cccc(-c8ccccc8)c7)cc6)c5ccc43)cc21]

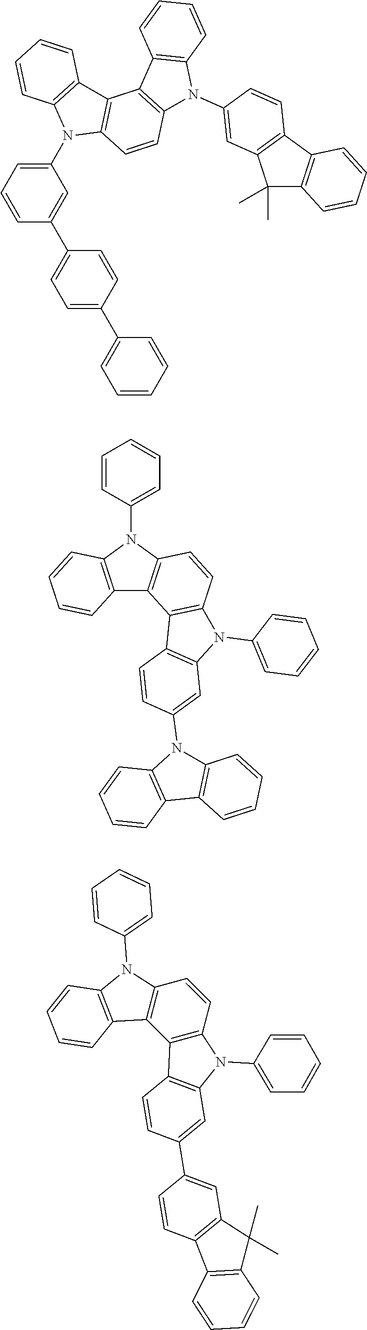

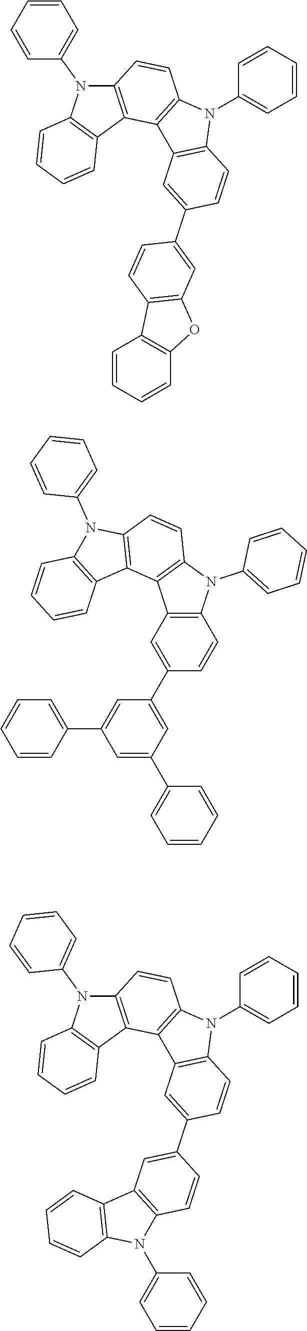 [SMILES: c1ccc(-n2c3ccccc3c3c4c5cc(-c6ccc7c(c6)oc6ccccc67)ccc5n(-c5ccccc5)c4ccc32)cc1 ; c1ccc(-c2cc(-c3ccccc3)cc(-c3ccc4c(c3)c3c5c6ccccc6n(-c6ccccc6)c5ccc3n4-c3ccccc3)c2)cc1 ; c1ccc(-n2c3ccccc3c3cc(-c4ccc5c(c4)c4c6c7ccccc7n(-c7ccccc7)c6ccc4n5-c4ccccc4)ccc32)cc1]

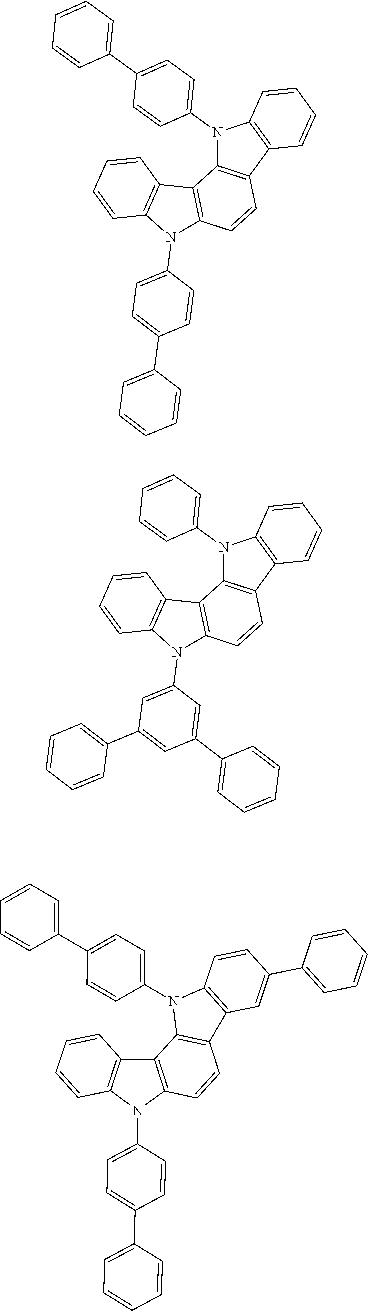 [SMILES: c1ccc(-c2ccc(-n3c4ccccc4c4c3ccc3c5ccccc5n(-c5ccc(-c6ccccc6)cc5)c34)cc2)cc1 ; c1ccc(-c2cc(-c3ccccc3)cc(-n3c4ccccc4c4c3ccc3c5ccccc5n(-c5ccccc5)c34)c2)cc1 ; c1ccc(-c2ccc(-n3c4ccccc4c4c3ccc3c5cc(-c6ccccc6)ccc5n(-c5ccc(-c6ccccc6)cc5)c34)cc2)cc1]

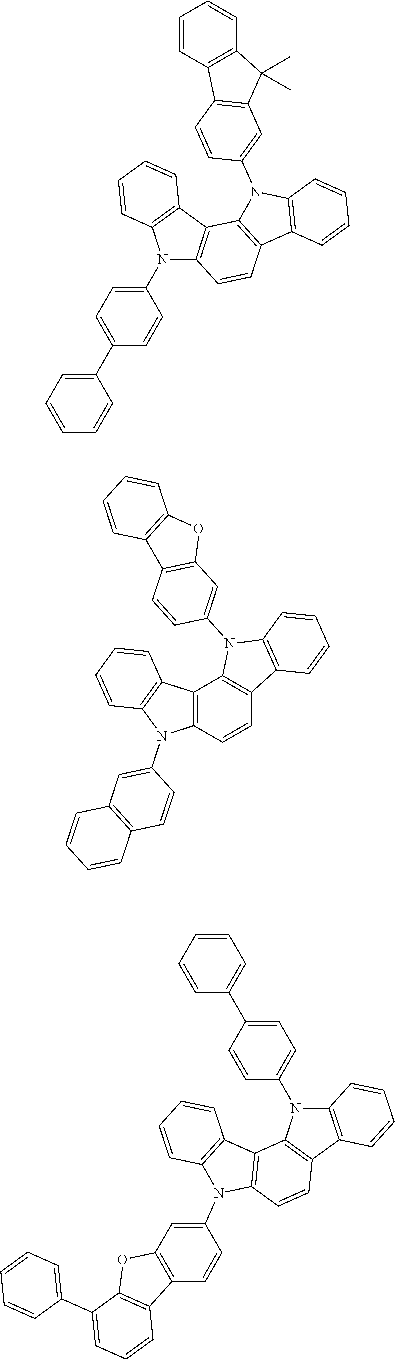 [SMILES: CC1(C)c2ccccc2-c2ccc(-n3c4ccccc4c4ccc5c(c6ccccc6n5-c5ccc(-c6ccccc6)cc5)c43)cc21 ; c1ccc2cc(-n3c4ccccc4c4c3ccc3c5ccccc5n(-c5ccc6c(c5)oc5ccccc56)c34)ccc2c1 ; c1ccc(-c2ccc(-n3c4ccccc4c4ccc5c(c6ccccc6n5-c5ccc6c(c5)oc5c(-c7ccccc7)cccc56)c43)cc2)cc1]

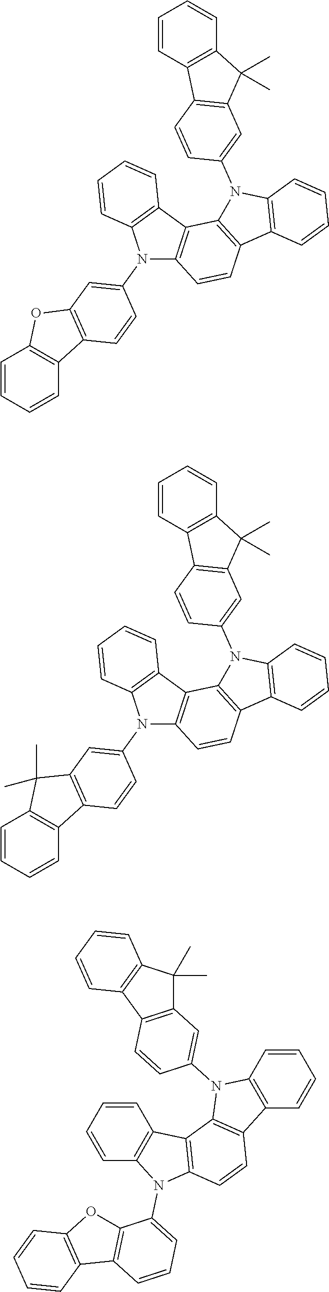 [SMILES: CC1(C)c2ccccc2-c2ccc(-n3c4ccccc4c4ccc5c(c6ccccc6n5-c5ccc6c(c5)oc5ccccc56)c43)cc21 ; CC1(C)c2ccccc2-c2ccc(-n3c4ccccc4c4c3ccc3c5ccccc5n(-c5ccc6c(c5)C(C)(C)c5ccccc5-6)c34)cc21 ; CC1(C)c2ccccc2-c2ccc(-n3c4ccccc4c4ccc5c(c6ccccc6n5-c5cccc6c5oc5ccccc56)c43)cc21]

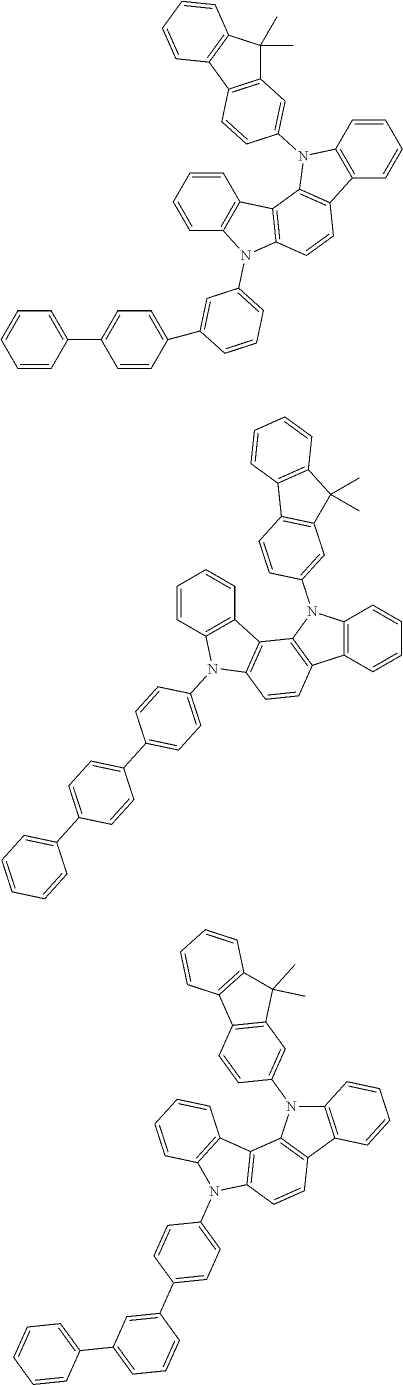 [SMILES: CC1(C)c2ccccc2-c2ccc(-n3c4ccccc4c4ccc5c(c6ccccc6n5-c5cccc(-c6ccc(-c7ccccc7)cc6)c5)c43)cc21 ; CC1(C)c2ccccc2-c2ccc(-n3c4ccccc4c4ccc5c(c6ccccc6n5-c5ccc(-c6ccc(-c7ccccc7)cc6)cc5)c43)cc21 ; CC1(C)c2ccccc2-c2ccc(-n3c4ccccc4c4ccc5c(c6ccccc6n5-c5ccc(-c6cccc(-c7ccccc7)c6)cc5)c43)cc21]

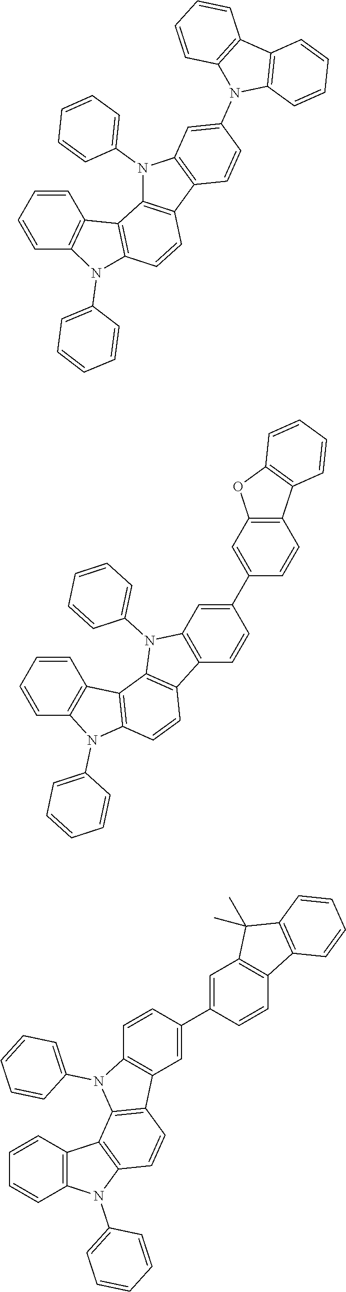 [SMILES: c1ccc(-n2c3ccccc3c3c2ccc2c4ccc(-n5c6ccccc6c6ccccc65)cc4n(-c4ccccc4)c23)cc1 ; c1ccc(-n2c3ccccc3c3c2ccc2c4ccc(-c5ccc6c(c5)oc5ccccc56)cc4n(-c4ccccc4)c23)cc1 ; CC1(C)c2ccccc2-c2ccc(-c3ccc4c(c3)c3ccc5c(c6ccccc6n5-c5ccccc5)c3n4-c3ccccc3)cc21]

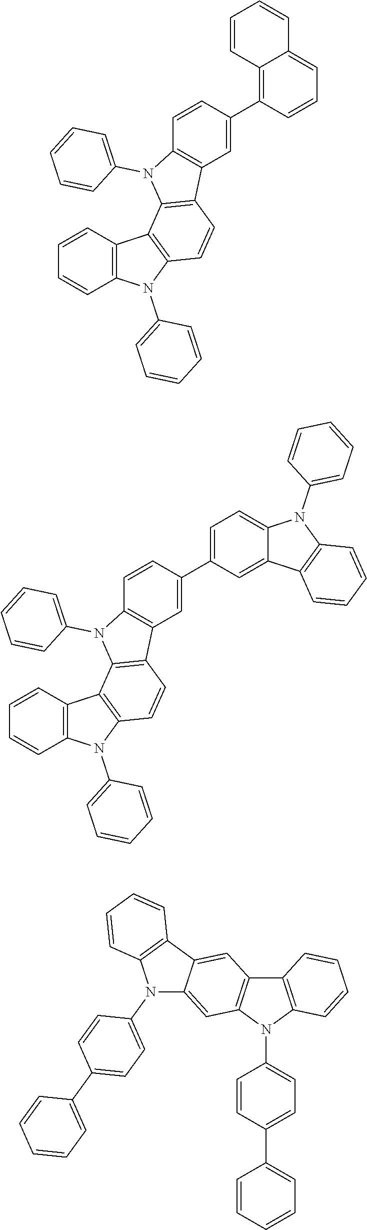 [SMILES: c1ccc(-n2c3ccccc3c3c2ccc2c4cc(-c5cccc6ccccc56)ccc4n(-c4ccccc4)c23)cc1 ; c1ccc(-n2c3ccccc3c3cc(-c4ccc5c(c4)c4ccc6c(c7ccccc7n6-c6ccccc6)c4n5-c4ccccc4)ccc32)cc1 ; c1ccc(-c2ccc(-n3c4ccccc4c4cc5c6ccccc6n(-c6ccc(-c7ccccc7)cc6)c5cc43)cc2)cc1]

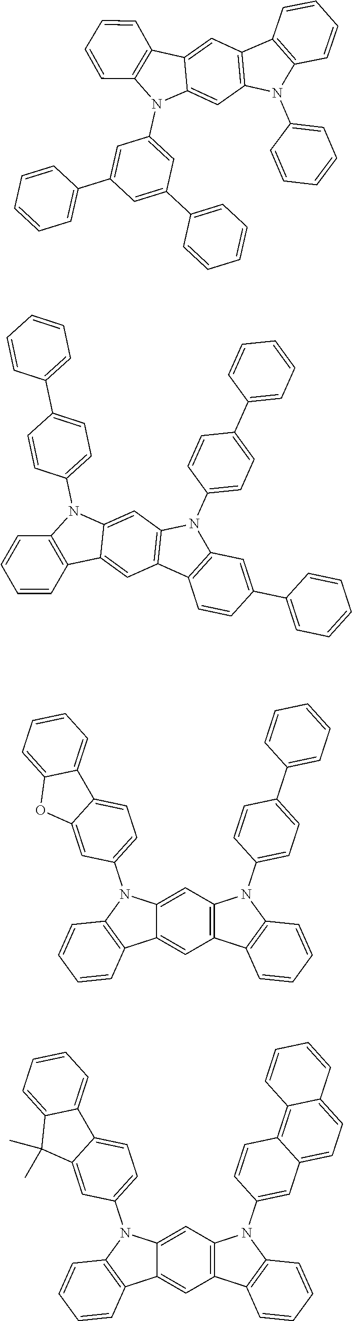 [SMILES: c1ccc(-c2cc(-c3ccccc3)cc(-n3c4ccccc4c4cc5c6ccccc6n(-c6ccccc6)c5cc43)c2)cc1 ; c1ccc(-c2ccc(-n3c4ccccc4c4cc5c6ccc(-c7ccccc7)cc6n(-c6ccc(-c7ccccc7)cc6)c5cc43)cc2)cc1 ; c1ccc(-c2ccc(-n3c4ccccc4c4cc5c6ccccc6n(-c6ccc7c(c6)oc6ccccc67)c5cc43)cc2)cc1 ; CC1(C)c2ccccc2-c2ccc(-n3c4ccccc4c4cc5c6ccccc6n(-c6ccc7c(ccc8ccccc87)c6)c5cc43)cc21]

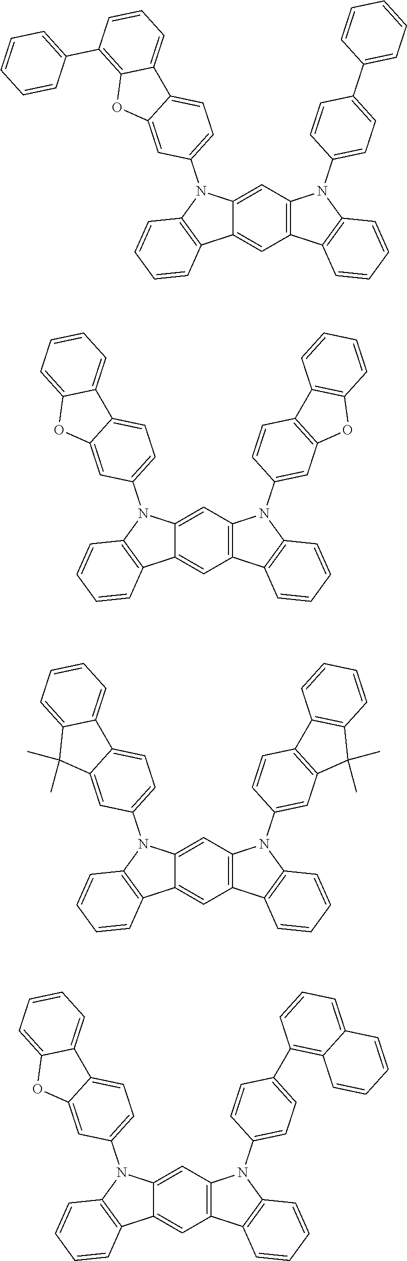

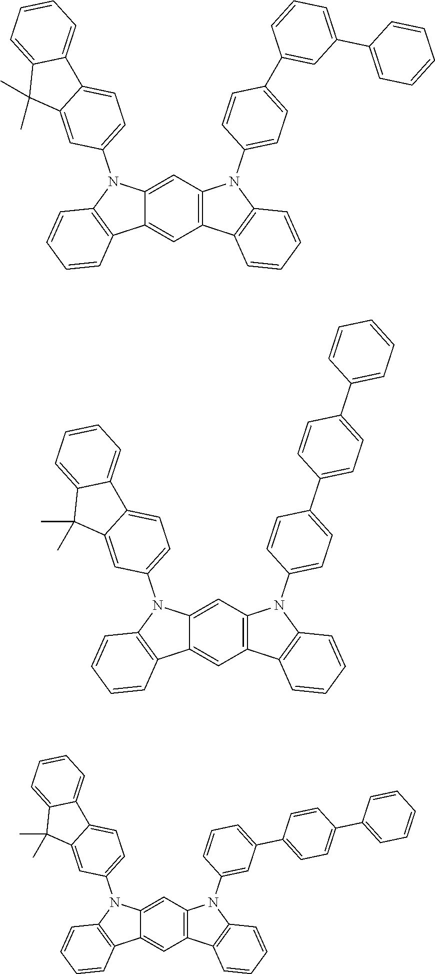 [SMILES: CC1(C)c2ccccc2-c2ccc(-n3c4ccccc4c4cc5c6ccccc6n(-c6ccc(-c7cccc(-c8ccccc8)c7)cc6)c5cc43)cc21 ; CC1(C)c2ccccc2-c2ccc(-n3c4ccccc4c4cc5c6ccccc6n(-c6ccc(-c7ccc(-c8ccccc8)cc7)cc6)c5cc43)cc21 ; CC1(C)c2ccccc2-c2ccc(-n3c4ccccc4c4cc5c6ccccc6n(-c6cccc(-c7ccc(-c8ccccc8)cc7)c6)c5cc43)cc21]

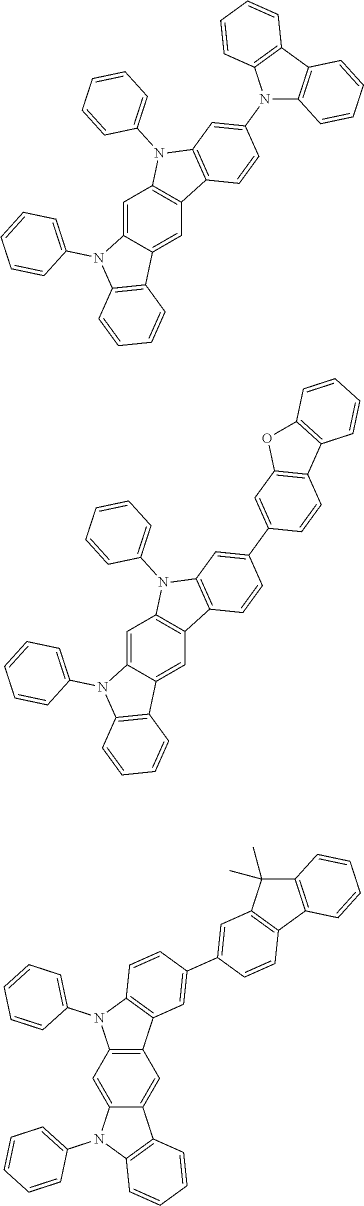 [SMILES: c1ccc(-n2c3ccccc3c3cc4c5ccc(-n6c7ccccc7c7ccccc76)cc5n(-c5ccccc5)c4cc32)cc1 ; c1ccc(-n2c3ccccc3c3cc4c5ccc(-c6ccc7c(c6)oc6ccccc67)cc5n(-c5ccccc5)c4cc32)cc1 ; CC1(C)c2ccccc2-c2ccc(-c3ccc4c(c3)c3cc5c6ccccc6n(-c6ccccc6)c5cc3n4-c3ccccc3)cc21]

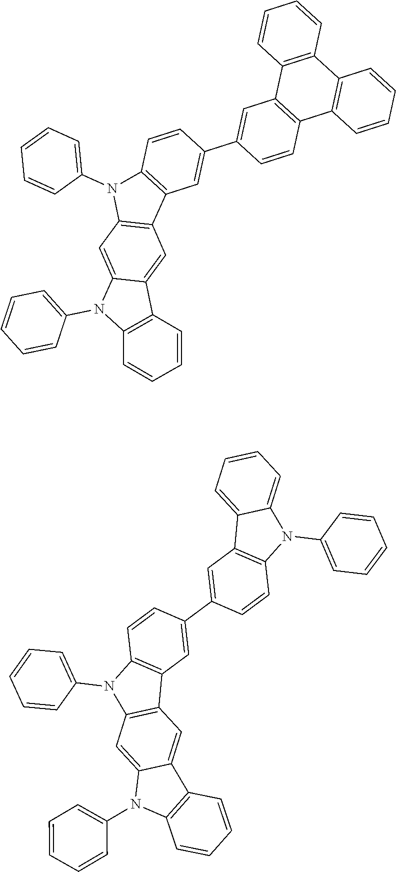 [SMILES: c1ccc(-n2c3ccccc3c3cc4c5cc(-c6ccc7c8ccccc8c8ccccc8c7c6)ccc5n(-c5ccccc5)c4cc32)cc1 ; c1ccc(-n2c3ccccc3c3cc(-c4ccc5c(c4)c4cc6c7ccccc7n(-c7ccccc7)c6cc4n5-c4ccccc4)ccc32)cc1]

The host material (triplet host), phosphorescent emitting material (triplet emitter), and TADF material are described in detail below (but not limited thereto).
1. Triplet Host

Examples of triplet host materials are not specially limited and any metal complex or organic compound may be used as the host material as long as its triplet energy is higher than that of the emitter, especially higher than that of the triplet emitter or a phosphorescent emitter. Examples of metal complexes that may be used as a triplet host include (but are not limited to) the following general structures.

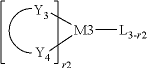

Where M3 is a metal; (Y₃-Y₄) is a bidentate ligand; Y₃ and Y₄ are each independently selected from C, N, O, P, or S; L is an auxiliary ligand; r2 is an integer with the value from 1 to the maximum coordination number of this metal.

In some embodiments, the metal complexes that can be used as the triplet host have the following form:

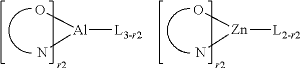

(O—N) is a bidentate ligand in which the metal is coordinated to the O atoms and the N atoms. r2 is an integer with a value from 1 to the maximum coordination number of this metal.

In some embodiments, M3 may be selected from Ir or Pt.

Examples of organic compounds that can be used as the triplet host are selected from: compounds containing cyclic aromatic hydrocarbon groups such as benzene, biphenyl, triphenylbenzene, benzofluorene; compounds containing aromatic heterocyclic groups such as dibenzothiophene, dibenzofuran, dibenzoselenophene, furan, thiophene, benzofuran, benzothiophene, benzoselenophene, carbazole, dibenzocarbazole, indole carbazole, pyridine indole, pyrrole dipyridine, pyrazole, imidazole, triazole, oxazole, thiazole, oxadiazole, oxatriazole, dioxazole, thiadiazole, pyridine, pyridazine, pyrimidine, pyrazine, triazines, oxazine, oxothiazine, oxadiazine, indole, benzimidazole, indazole, oxazole, dibenzoxazole, benzisoxazole, benzothiazole, quinoline, isoquinoline, cinnoline, quinazoline, quinoxaline, naphthalene, phthalide, pteridine, xanthene, acridine, phenazine, phenothiazine, Phenoxazine, benzofuranopyridine, furanopyridine, benzothienopyridine, thienopyridine, benzoselenophene pyridine or selenophene benzodipyridine; and groups containing 2 to 10 ring structures, which may be the same or different types of cyclic aromatic hydrocarbon groups or aromatic heterocyclic groups, linked to each other directly or by at least one of the following groups, such as oxygen atoms, nitrogen atoms, sulfur atoms, silicon atoms, phosphorus atoms, boron atoms, chain structural units and aliphatic ring groups, where each Ar can be further substituted and the substituents may selected from hydrogen, deuterium, cyano, halogen, alkyl, alkoxy, amino, alkene, alkyne, aryl, heteroalkyl, aryl or heteroaryl.

In some embodiments, the triplet host material may be selected from a compound comprising at least one of the following groups.

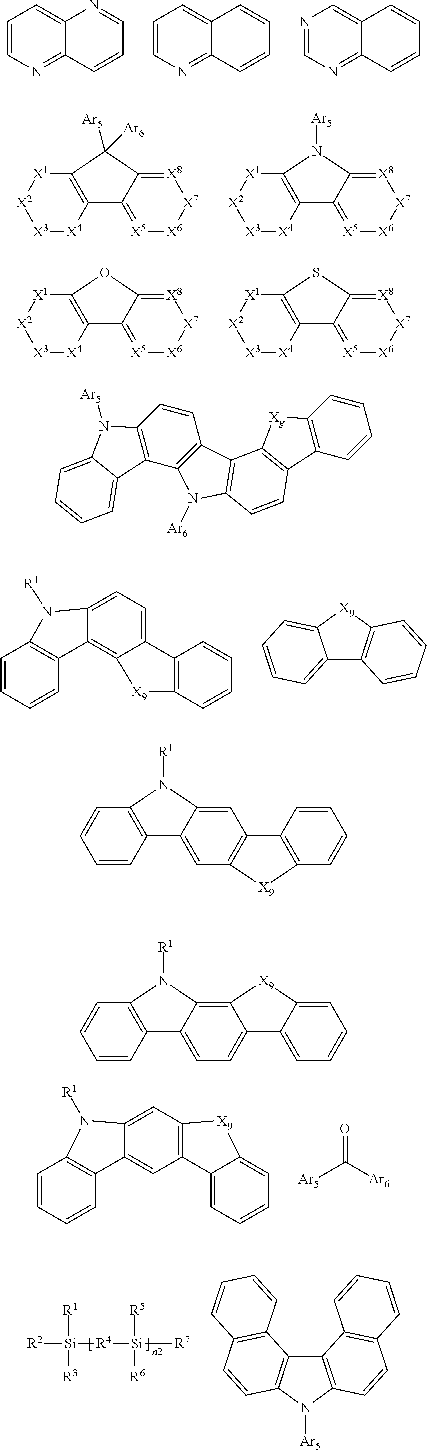

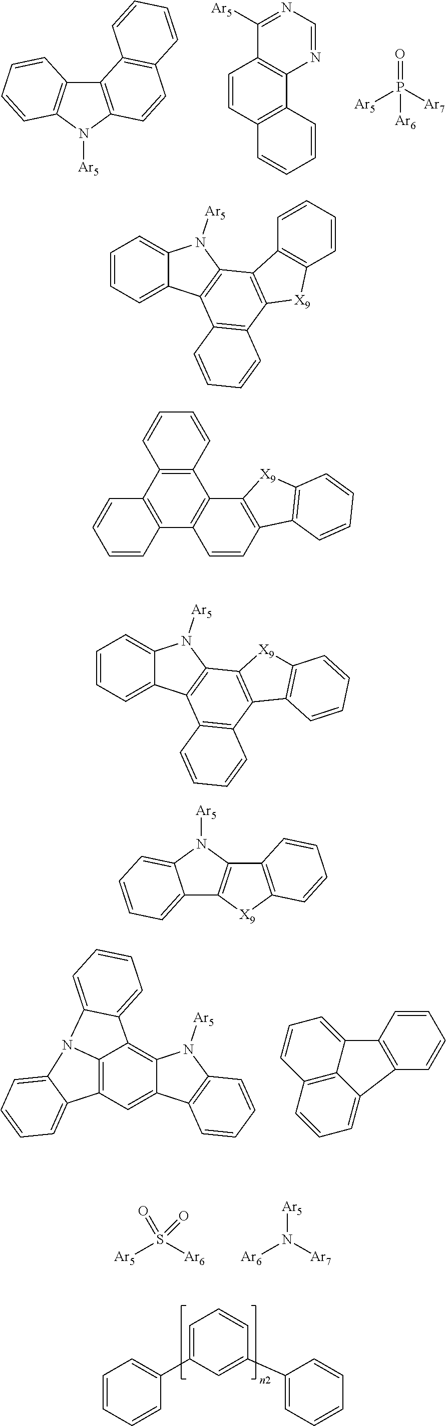

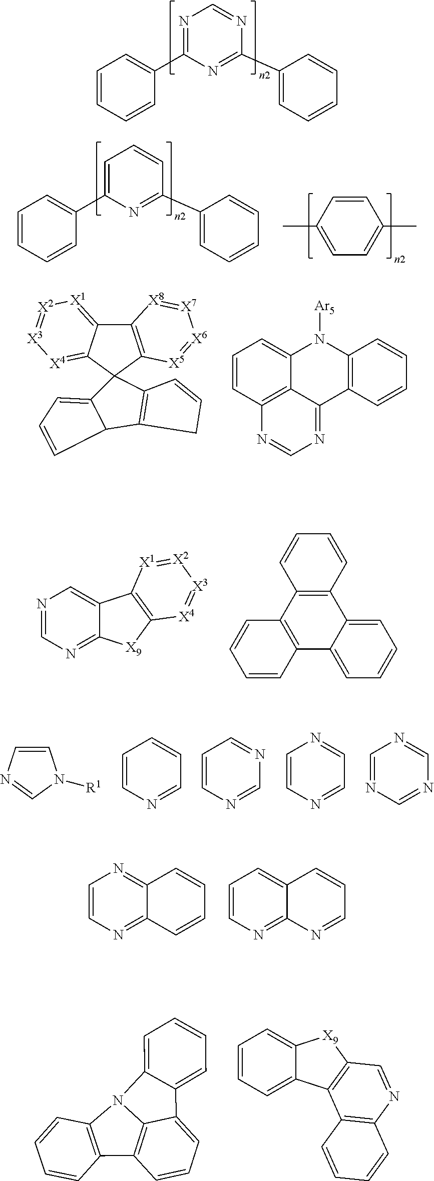

X¹-X⁸ and X₉ is CR⁸R⁹ or NR¹⁰; Y is CR⁸R⁹, NR¹⁰, O, or S; n2 is defined as the above-mentioned n; Ar₅-Ar₇ are identically defined as defined as the above-mentioned Ar₁; R¹ to R¹⁰ are identically defined as the above-mentioned R₁.

Examples of suitable triplet host materials are listed below, but not limited to:

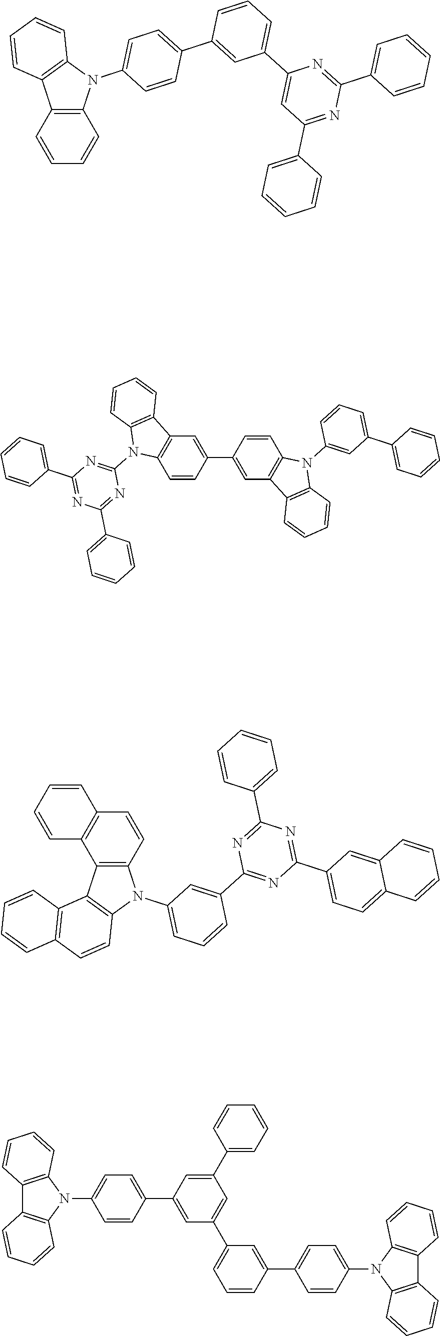 [SMILES: c1ccc(-c2cc(-c3cccc(-c4ccc(-n5c6ccccc6c6ccccc65)cc4)c3)nc(-c3ccccc3)n2)cc1 ; c1ccc(-c2cccc(-n3c4ccccc4c4cc(-c5ccc6c(c5)c5ccccc5n6-c5nc(-c6ccccc6)nc(-c6ccccc6)n5)ccc43)c2)cc1 ; c1ccc(-c2nc(-c3cccc(-n4c5ccc6ccccc6c5c5c6ccccc6ccc54)c3)nc(-c3ccc4ccccc4c3)n2)cc1 ; c1ccc(-c2cc(-c3ccc(-n4c5ccccc5c5ccccc54)cc3)cc(-c3cccc(-c4ccc(-n5c6ccccc6c6ccccc65)cc4)c3)c2)cc1]

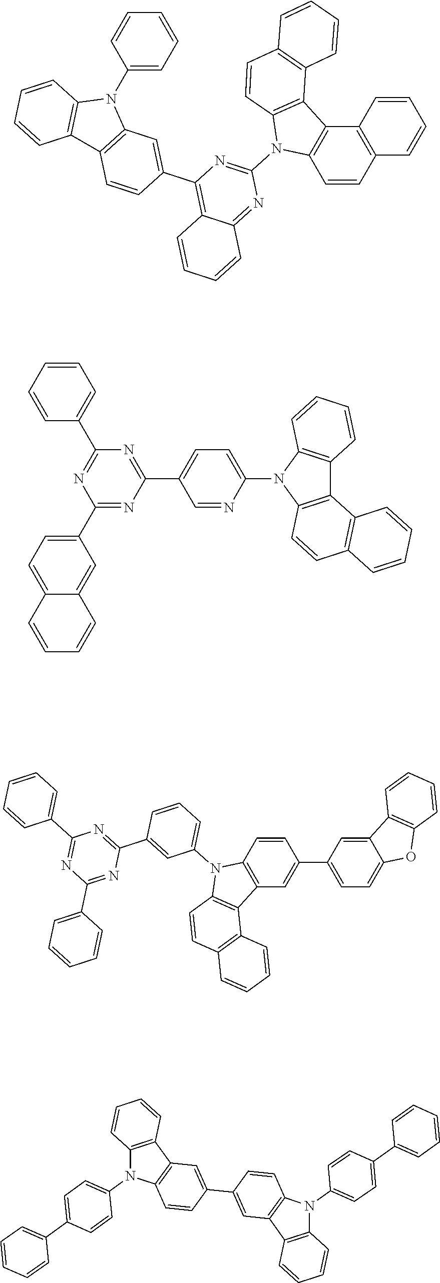 [SMILES: c1ccc(-n2c3ccccc3c3ccc(-c4nc(-n5c6ccc7ccccc7c6c6c7ccccc7ccc65)nc5ccccc45)cc32)cc1 ; c1ccc(-c2nc(-c3ccc(-n4c5ccccc5c5c6ccccc6ccc54)nc3)nc(-c3ccc4ccccc4c3)n2)cc1 ; c1ccc(-c2nc(-c3ccccc3)nc(-c3cccc(-n4c5ccc(-c6ccc7oc8ccccc8c7c6)cc5c5c6ccccc6ccc54)c3)n2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

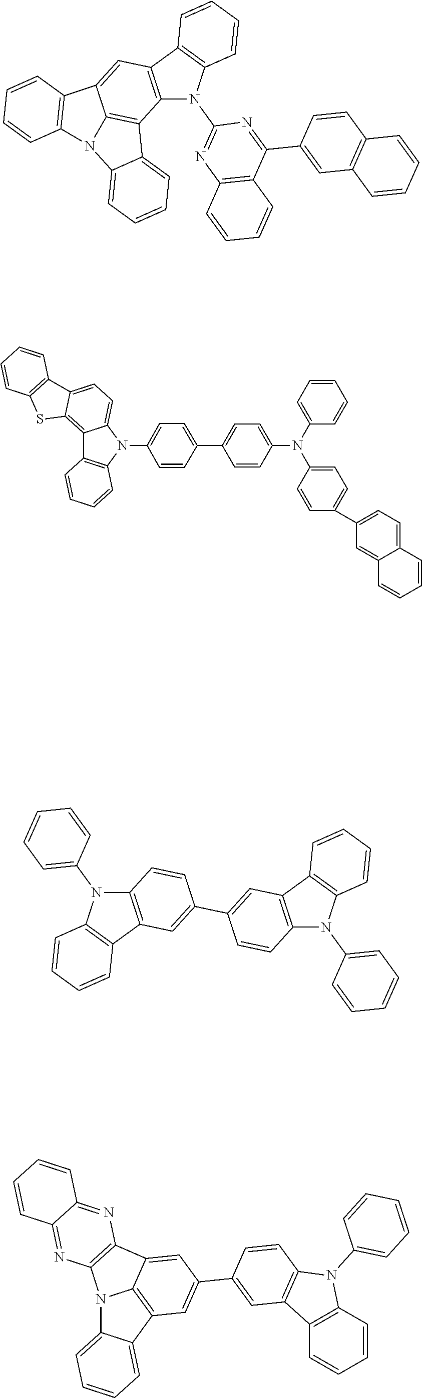 [SMILES: c1ccc2cc(-c3nc(-n4c5ccccc5c5cc6c7ccccc7n7c8ccccc8c(c54)c67)nc4ccccc34)ccc2c1 ; c1ccc(N(c2ccc(-c3ccc(-n4c5ccccc5c5c6sc7ccccc7c6ccc54)cc3)cc2)c2ccc(-c3ccc4ccccc4c3)cc2)cc1 ; c1ccc(-n2c3ccccc3c3cc(-c4ccc5c(c4)c4ccccc4n5-c4ccccc4)ccc32)cc1 ; c1ccc(-n2c3ccccc3c3cc(-c4cc5c6ccccc6n6c7nc8ccccc8nc7c(c4)c56)ccc32)cc1]

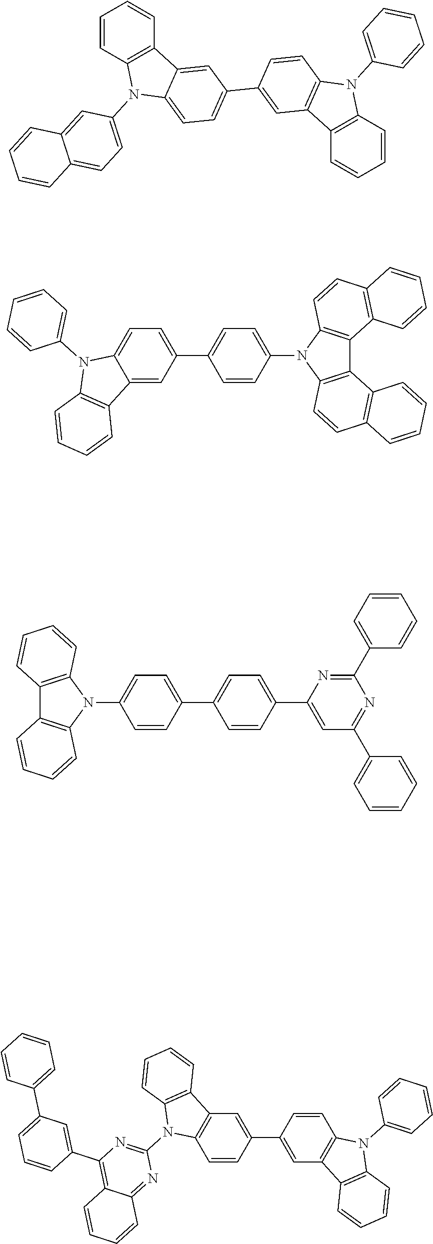 [SMILES: c1ccc(-n2c3ccccc3c3cc(-c4ccc5c(c4)c4ccccc4n5-c4ccc5ccccc5c4)ccc32)cc1 ; c1ccc(-n2c3ccccc3c3cc(-c4ccc(-n5c6ccc7ccccc7c6c6c7ccccc7ccc65)cc4)ccc32)cc1 ; c1ccc(-c2cc(-c3ccc(-c4ccc(-n5c6ccccc6c6ccccc65)cc4)cc3)nc(-c3ccccc3)n2)cc1 ; c1ccc(-c2cccc(-c3nc(-n4c5ccccc5c5cc(-c6ccc7c(c6)c6ccccc6n7-c6ccccc6)ccc54)nc4ccccc34)c2)cc1]

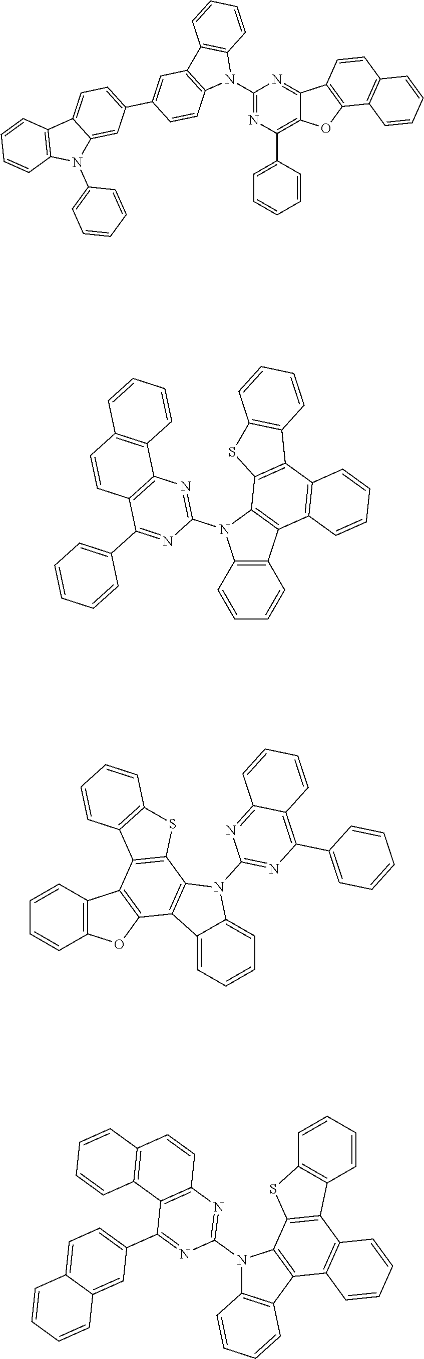 [SMILES: c1ccc(-c2nc(-n3c4ccccc4c4cc(-c5ccc6c7ccccc7n(-c7ccccc7)c6c5)ccc43)nc3c2oc2c4ccccc4ccc32)cc1 ; c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5oc6ccccc6c5c5c6ccccc6sc5c43)nc3ccccc23)cc1 ; c1ccc2cc(-c3nc(-n4c5ccccc5c5c6ccccc6c6c7ccccc7sc6c54)nc4ccc5ccccc5c34)ccc2c1]

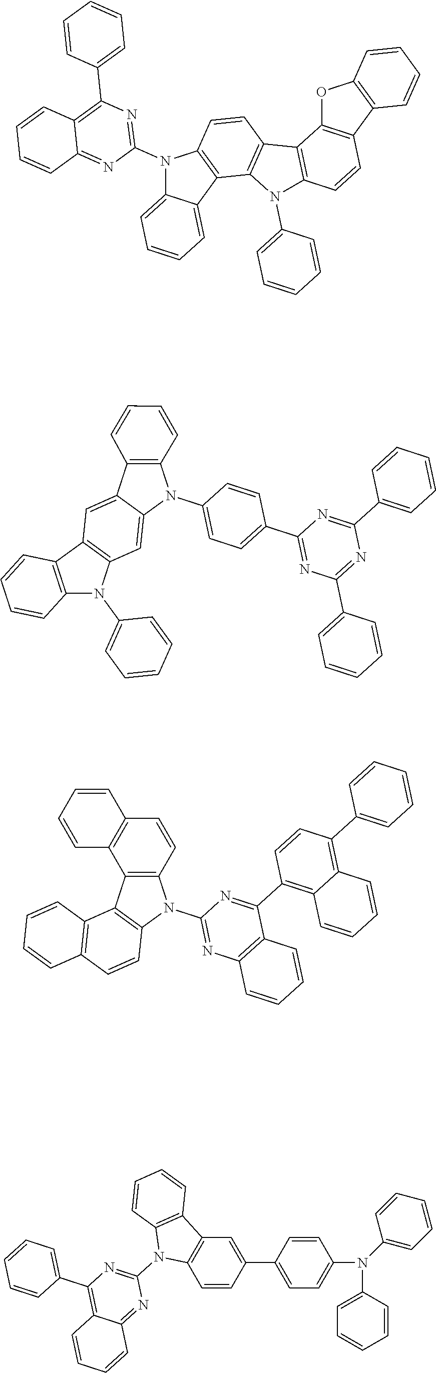 [SMILES: c1ccc(-c2nc(-n3c4ccccc4c4c3ccc3c5c6oc7ccccc7c6ccc5n(-c5ccccc5)c34)nc3ccccc23)cc1 ; c1ccc(-c2nc(-c3ccccc3)nc(-c3ccc(-n4c5ccccc5c5cc6c7ccccc7n(-c7ccccc7)c6cc54)cc3)n2)cc1 ; c1ccc(-c2ccc(-c3nc(-n4c5ccc6ccccc6c5c5c6ccccc6ccc54)nc4ccccc34)c3ccccc23)cc1 ; c1ccc(-c2nc(-n3c4ccccc4c4cc(-c5ccc(N(c6ccccc6)c6ccccc6)cc5)ccc43)nc3ccccc23)cc1]

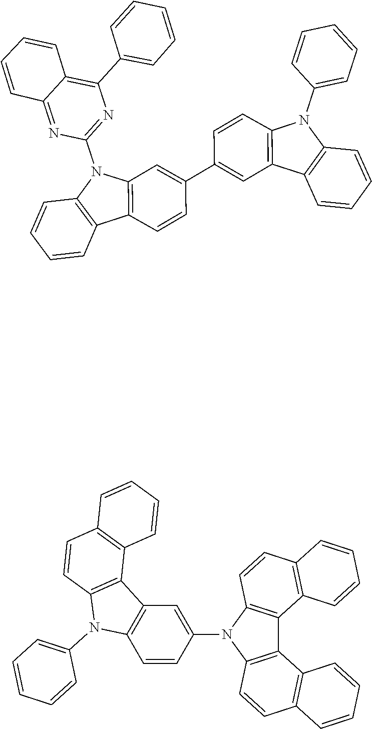 [SMILES: c1ccc(-c2nc(-n3c4ccccc4c4ccc(-c5ccc6c(c5)c5ccccc5n6-c5ccccc5)cc43)nc3ccccc23)cc1 ; c1ccc(-n2c3ccc(-n4c5ccc6ccccc6c5c5c6ccccc6ccc54)cc3c3c4ccccc4ccc32)cc1]

2. Triplet Emitter

The triplet emitter is also called a phosphorescent emitter. In some embodiments, the triplet emitter is a metal complex of formula M(L)ₙ, where M may be a metal atom; L may be a same or different organic ligand each time it is present, and may be bonded or coordinated to the metal atom M at one or more positions; n is an integer greater than 1, preferably is 1, 2, 3, 4, 5, or 6. Alternatively, these metal complexes may be attached to a polymer by one or more positions, most preferably through an organic ligand.

In some embodiments, the metal atom M may be selected from the group consisting of transition metal elements or lanthanides or actinides, preferably Ir, Pt, Pd, Au, Rh, Ru, Os, Sm, Eu, Gd, Tb, Dy, Re, Cu, or Ag, and particularly preferably Os, Ir, Ru, Rh, Re, Pd, Au, or Pt.

Preferably, the triplet emitter comprises a chelating ligand (i.e., a ligand) coordinating to the metal by at least two bonding sites, and it is particularly preferred that the triplet emitter comprises two or three identical or different bidentate or multidentate ligand. Chelating ligands help to improve stability of metal complexes.

Examples of organic ligands may be selected from the group consisting of phenylpyridine derivatives, 7,8-benzoquinoline derivatives, 2 (2-thienyl) pyridine derivatives, 2 (1-naphthyl) pyridine derivatives, or 2 phenylquinoline derivatives. All of these organic ligands may be substituted, for example, substituted with fluoromethyl or trifluoromethyl. The auxiliary ligand may be preferably selected from acetylacetonate or picric acid.

In some embodiments, the metal complex which may be used as the triplet emitter may have the following form:

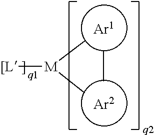

Where M is a metal selected from the group consisting of transition metal elements or lanthanides or actinides, and particularly preferably Ir, Pt, Au.

Ar¹ may be the same or different cyclic group each time it is present, which comprises a donor atom, that is an atom with a lone pair of electrons, such as nitrogen atom or phosphorus atom, which is coordinated to the metal through its ring group; Ar² may be the same or different cyclic group comprising a carbon atom and is coordinated to the metal through its ring group; Ar¹ and Ar² are covalently bonded together, where each of them may carry one or more substituents which may also be joined together by substituents; L′ may be the same or different at each occurrence and is a bidentate chelating ligand, and most preferably a monoanionic bidentate chelating ligand; q1 is 0, 1, 2, or 3, preferably is 2 or 3; q2 is 0, 1, 2, or 3, and preferably is 0 or 1.

Examples of triplet emitter materials that are extremely useful may be found in the following patent documents and references: WO200070655, WO200141512, WO200202714, WO200215645, EP1191613, EP1191612, EP1191614, WO2005033244, WO2005019373, US20050258742, WO2009146770, WO2010015307, WO2010031485, WO2010054731, WO2010054728, WO2010086089, WO2010099852, WO2010102709, US20070087219A1, US20090061681A1, US20010053462A1, Baldo, Thompson et al. Nature, 403, (2000), 750-753, Adachi et al. Appl. Phys. Lett., 78 (2001), 1622-1624, J. Kido et al. Appl. Phys. Lett., 65 (1994), 2124, Kido et al. Chem. Lett., 657, 1990, US20070252517A1, Johnson et al., JACS, 105, 1983, 1795, Wrighton, JACS, 96, 1974, 998, Ma et al., Synth. Metals, 94, 1998, 245, U.S. Pat. Nos. 6,824,895, 7,029,766, 6,835,469, 6,830,828, US20010053462A1, WO2007095118A1, US2012004407A1, WO2012007088A1, WO2012007087A1, WO2012007086A1, US2008027220A1, WO2011157339A1, CN102282150A, WO2009118087A1, WO2013107487A1, WO2013094620A1, WO2013174471A1, WO2014031977A1, WO2014112450A1, WO2014007565A1, WO2014038456A1, WO2014024131A1, WO2014008982A1, WO201402337A1. The patent documents listed above are specially incorporated herein by reference in their entirety.

Examples of some suitable triplet emitters are listed below:

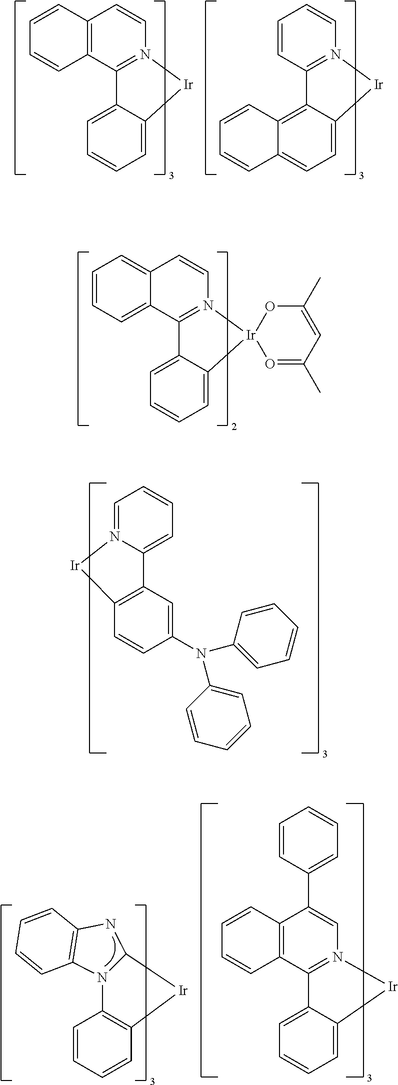

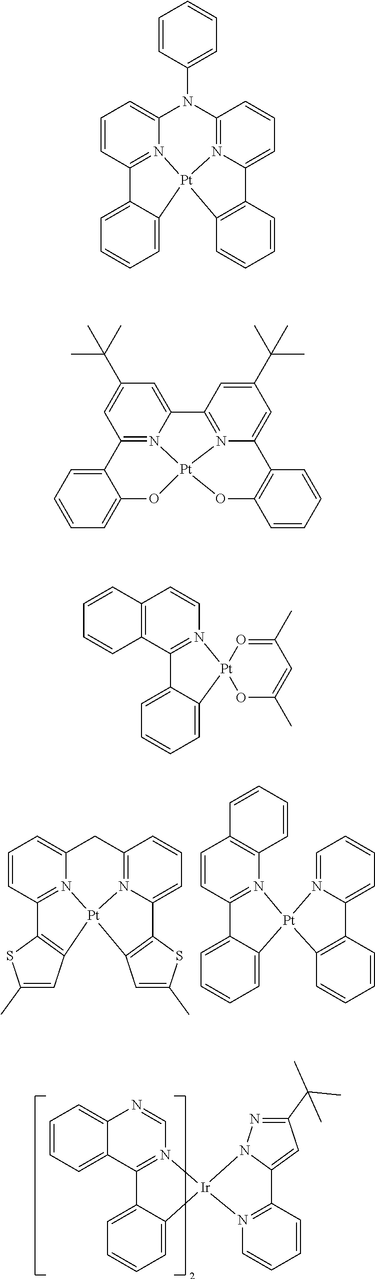

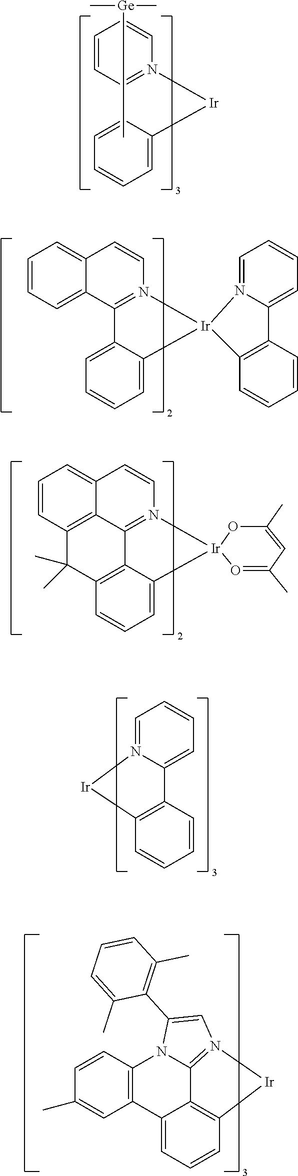

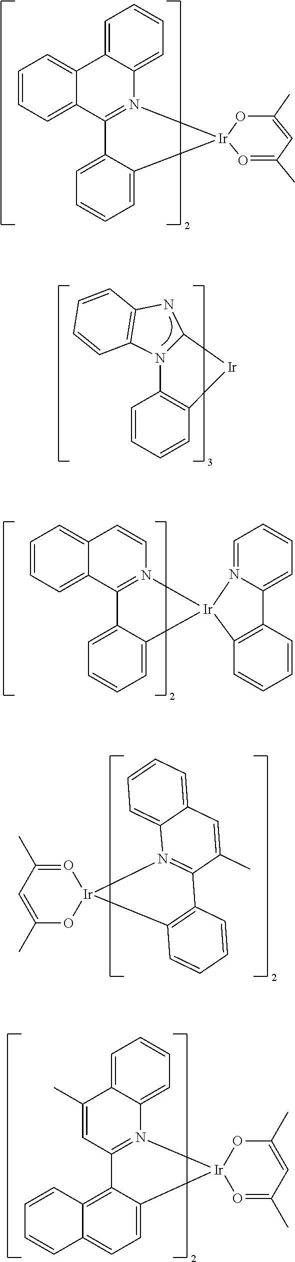

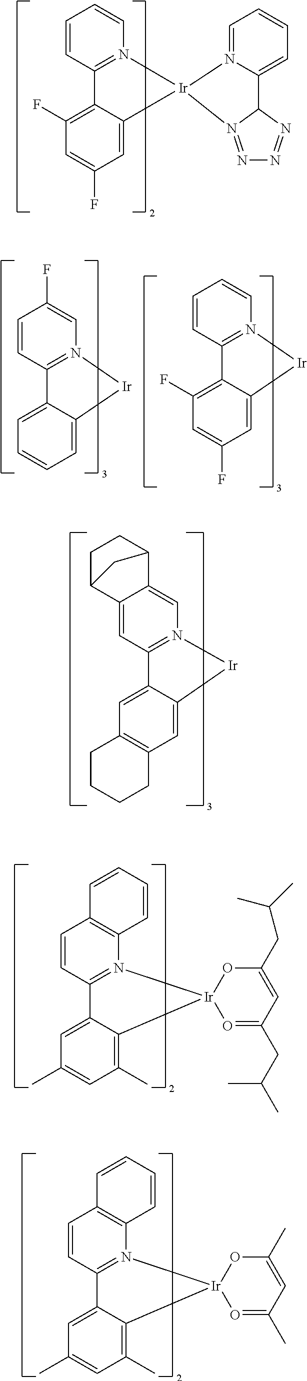

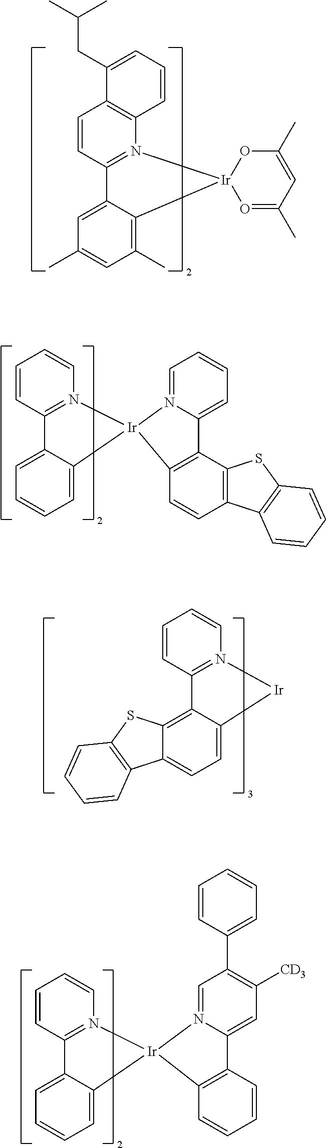

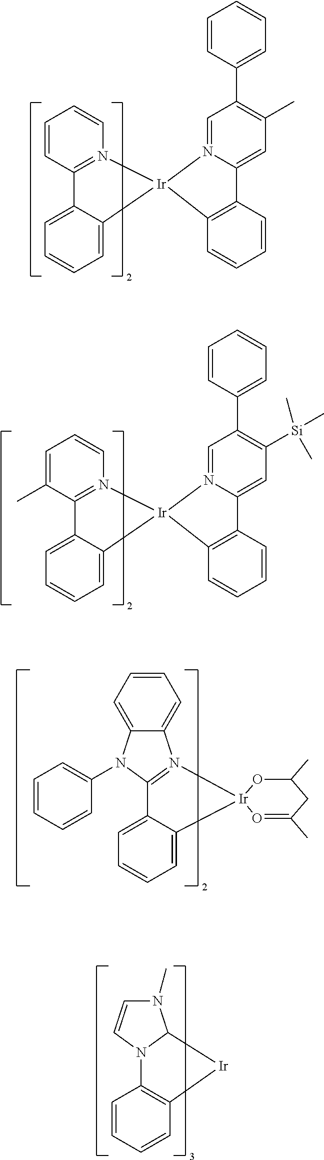

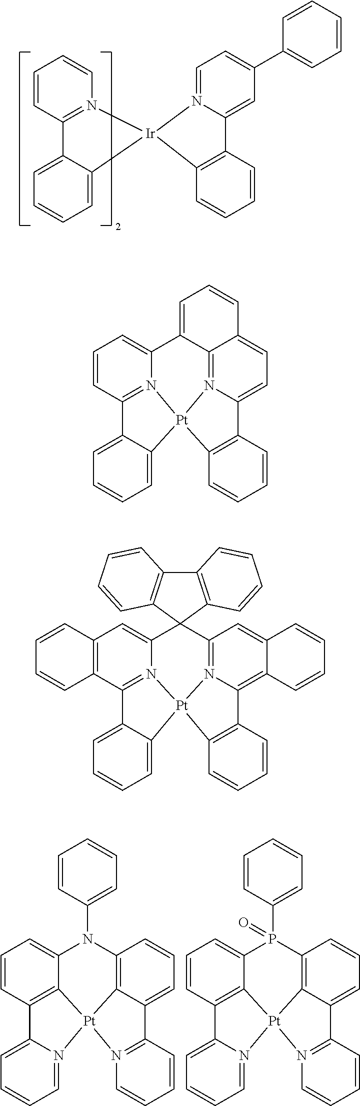

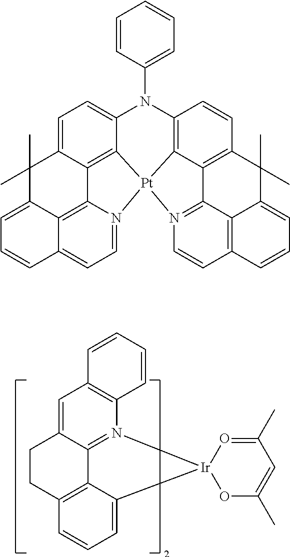

3. TADF Material

Conventional organic fluorescent materials can only emit light using 25% singlet excitonic luminescence formed by electrical excitation, and the devices have relatively low internal quantum efficiency (up to 25%). The phosphorescent material enhances the intersystem crossing due to the strong spin-orbit coupling of the heavy atom center, the singlet exciton and the triplet exciton luminescence formed by the electric excitation can be effectively utilized, so that the internal quantum efficiency of the device can reach 100%. However, the phosphorescent materials are expensive, the material stability is poor, and the device efficiency roll-off is a serious problem, which limit its application in OLED. Thermally-activated delayed fluorescent (TADF) materials are the third generation of organic emitting materials developed after organic fluorescent materials and organic phosphorescent materials. This type of material generally has a small singlet-triplet energy level difference (ΔEst), and triplet excitons can be converted to singlet excitons by intersystem crossing. This can make full use of the singlet excitons and triplet excitons formed under electric excitation. The device can achieve 100% internal quantum efficiency. Meanwhile, due to the controllable structure, stable property, low cost, TADF materials without precious metals have a wide application prospect in the OLED field.

The TADF material needs to have a small singlet-triplet energy level difference, preferably ΔEst<0.3 eV, more preferably ΔEst<0.2 eV, and most preferably ΔEst<0.1 eV. In some embodiments, the TADF material has a relatively small ΔEst. In some embodiments, the TADF has a high fluorescence quantum efficiency. Some TADF materials can be found in the following patent documents: CN103483332A, TW201309696A, TW201309778A, TW201343874A, TW201350558A, US20120217869A1, WO2013133359A1, WO2013154064A1, Adachi, et. al. Adv. Mater., 21, 2009, 4802, Adachi, et. al. Appl. Phys. Lett., 98, 2011, 083302, Adachi, et. al. Appl. Phys. Lett., 101, 2012, 093306, Adachi, et. al. Chem. Commun., 48, 2012, 11392, Adachi, et. al. Nature Photonics, 6, 2012, 253, Adachi, et. al. Nature, 492, 2012, 234, Adachi, et. al. J. Am. Chem. Soc., 134, 2012, 14706, Adachi, et. al. Angew. Chem. Int. Ed., 51, 2012, 11311, Adachi, et. al. Chem. Commun., 48, 2012, 9580, Adachi, et. al. Chem. Commun., 49, 2013, 10385, Adachi, et. al. Adv. Mater., 25, 2013, 3319, Adachi, et. al. Adv. Mater., 25, 2013, 3707, Adachi, et. al. Chem. Mater., 25, 2013, 3038, Adachi, et. al. Chem. Mater., 25, 2013,3766, Adachi, et. al. J. Mater. Chem. C., 1, 2013, 4599, Adachi, et. al. J. Phys. Chem. A., 117, 2013, 5607. The entire contents of the above listed patent or literature documents are hereby incorporated by reference.

Examples of some suitable TADF materials are listed below:

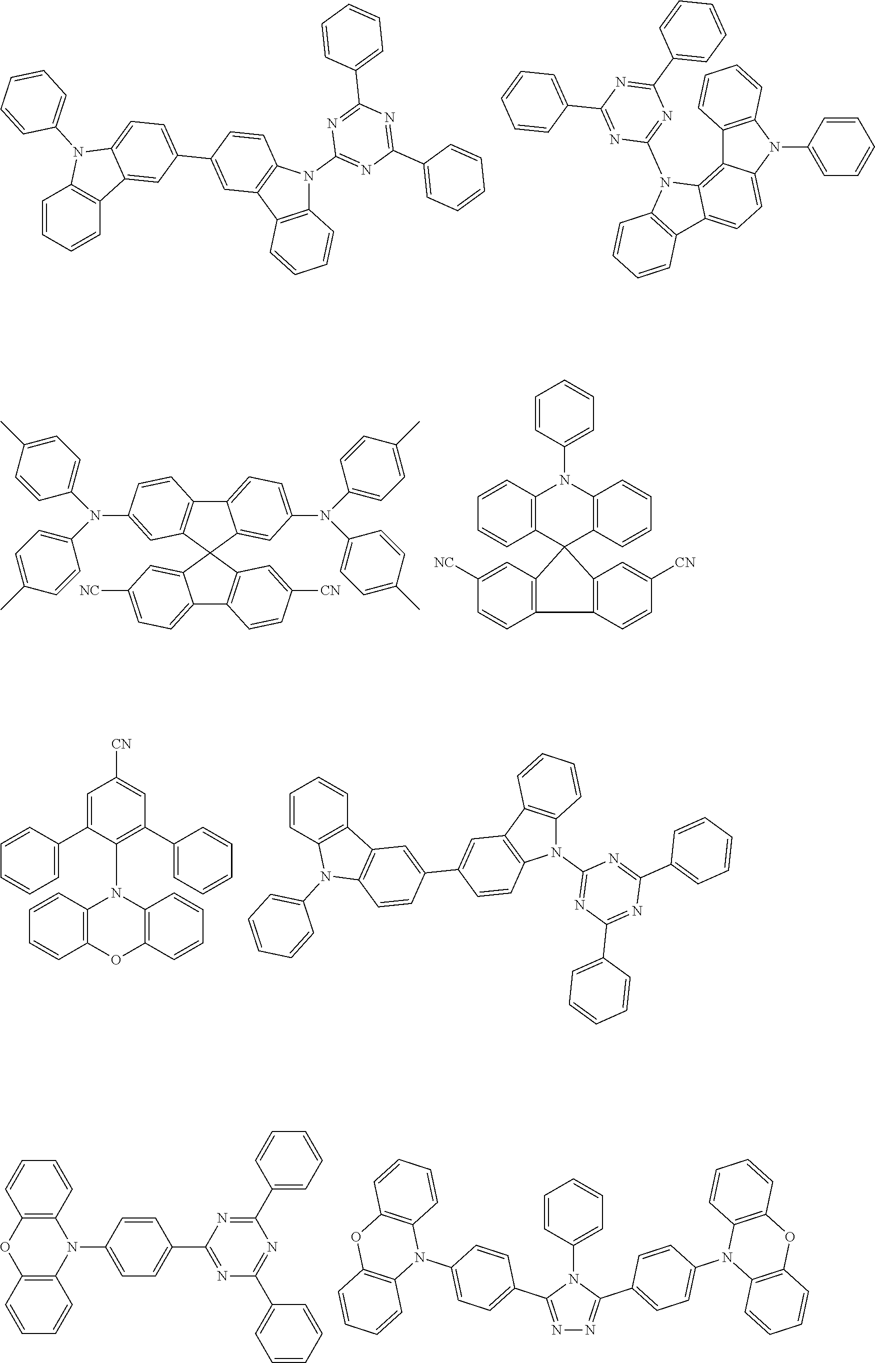

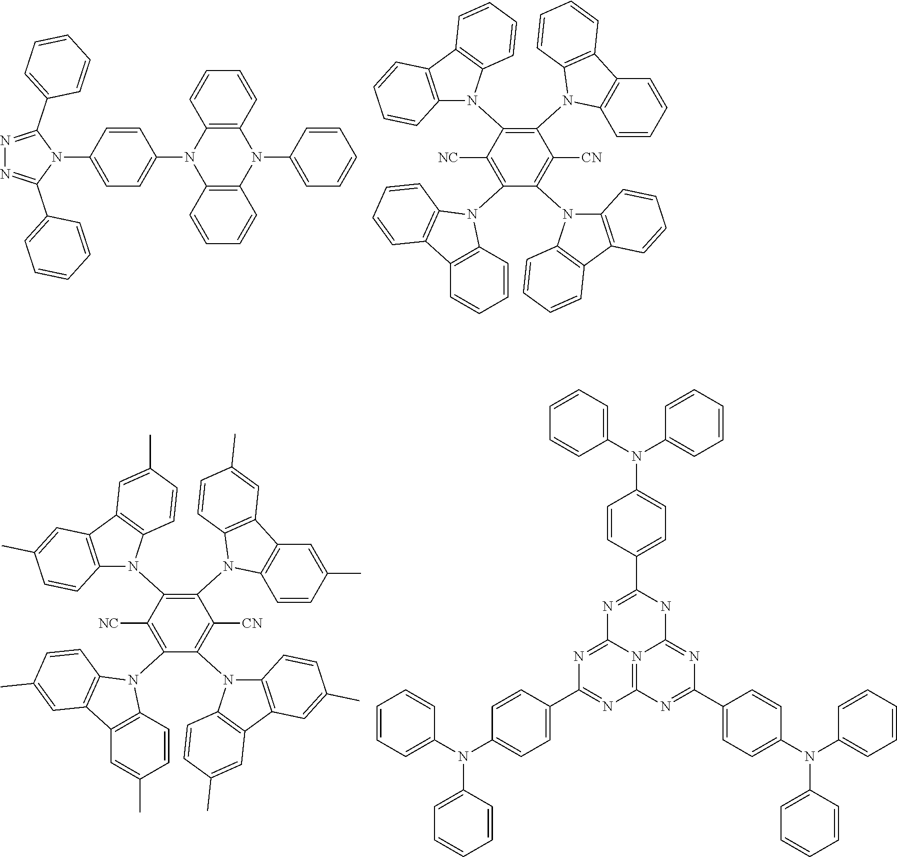

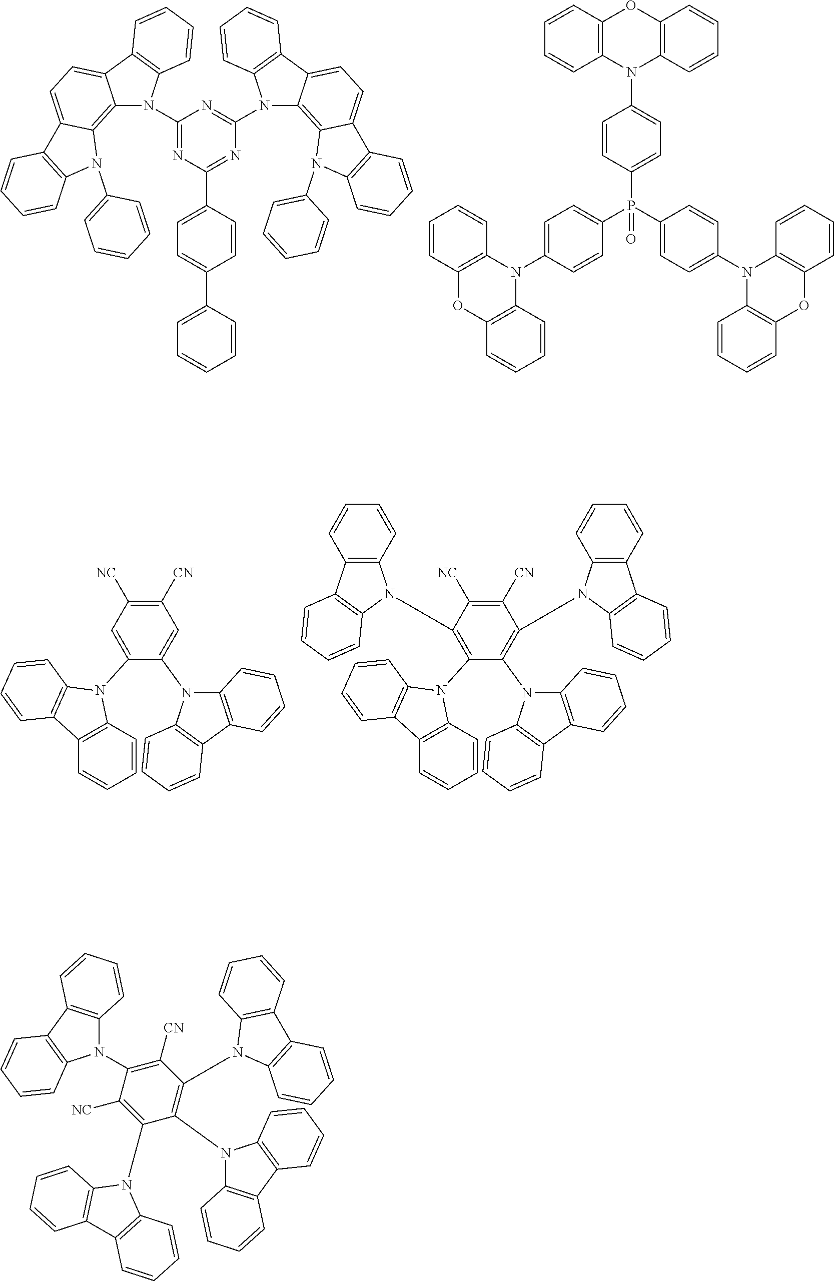

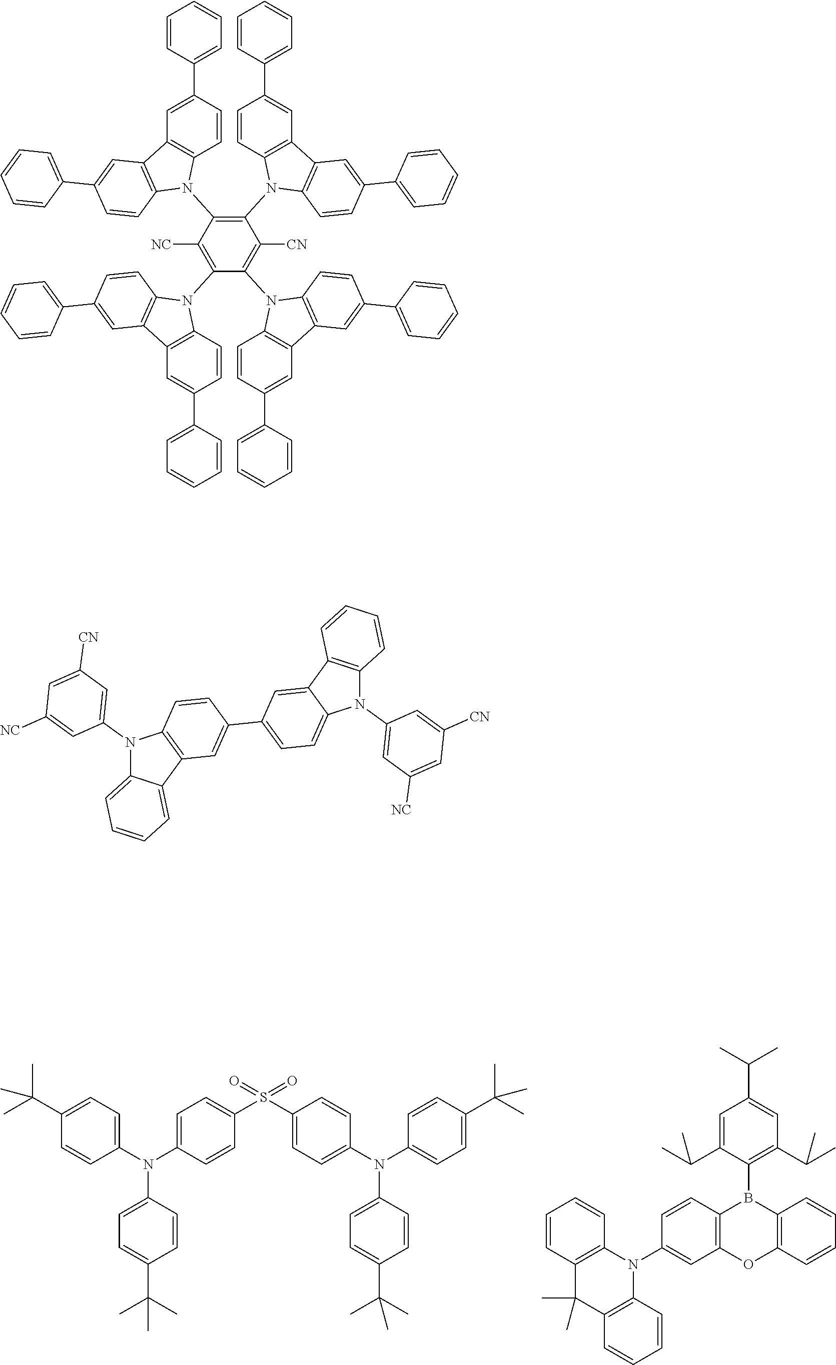 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(C#N)c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1cc(C#N)cc(-n2c3ccccc3c3cc(-c4ccc5c(c4)c4ccccc4n5-c4cc(C#N)cc(C#N)c4)ccc32)c1 ; CC(C)(C)c1ccc(N(c2ccc(C(C)(C)C)cc2)c2ccc(S(=O)(=O)c3ccc(N(c4ccc(C(C)(C)C)cc4)c4ccc(C(C)(C)C)cc4)cc3)cc2)cc1 ; CC(C)c1cc(C(C)C)c(B2c3ccccc3Oc3cc(N4c5ccccc5C(C)(C)c5ccccc54)ccc32)c(C(C)C)c1]

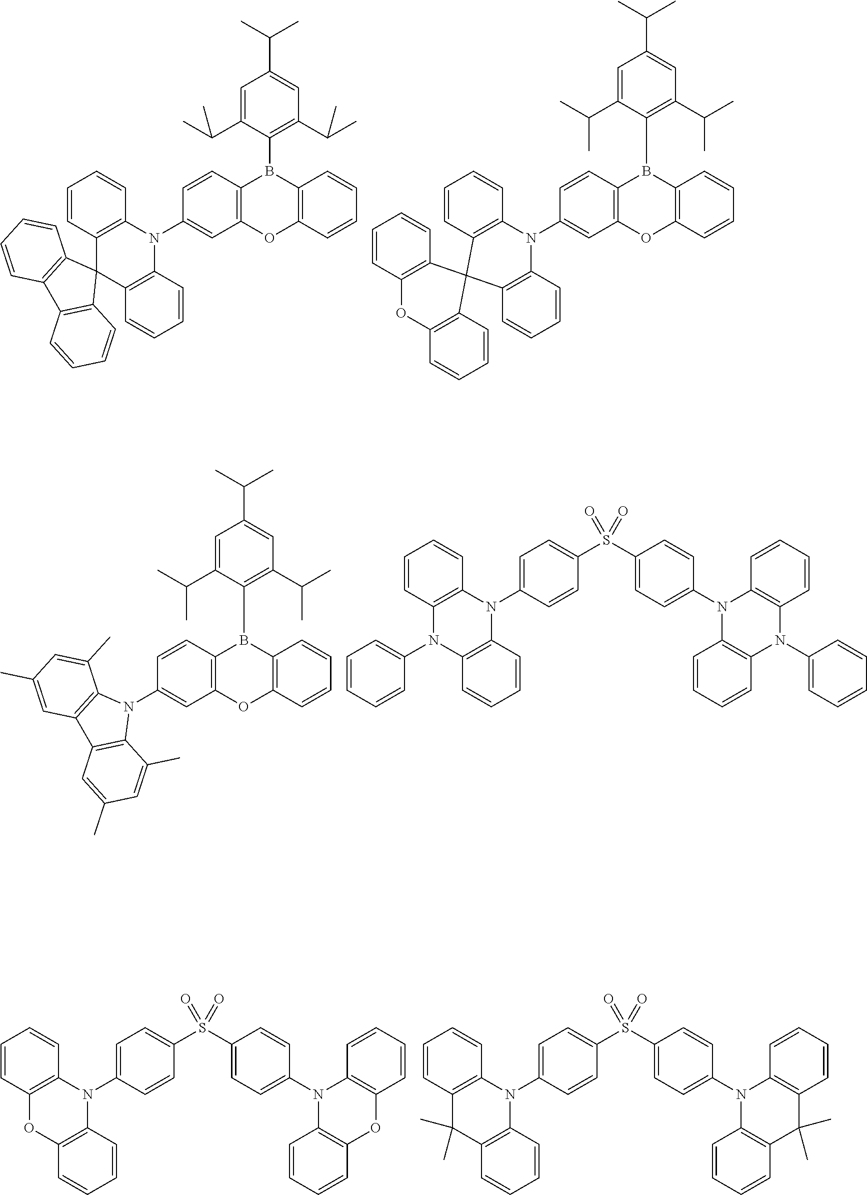

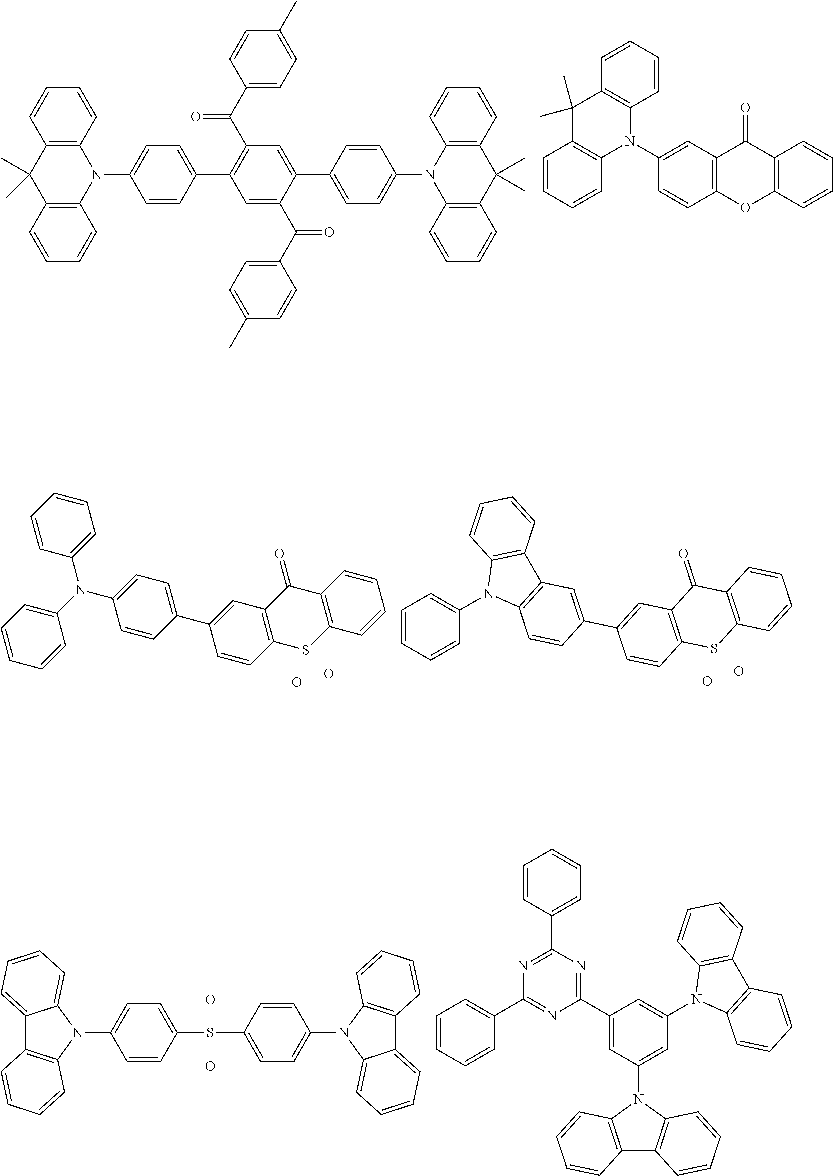

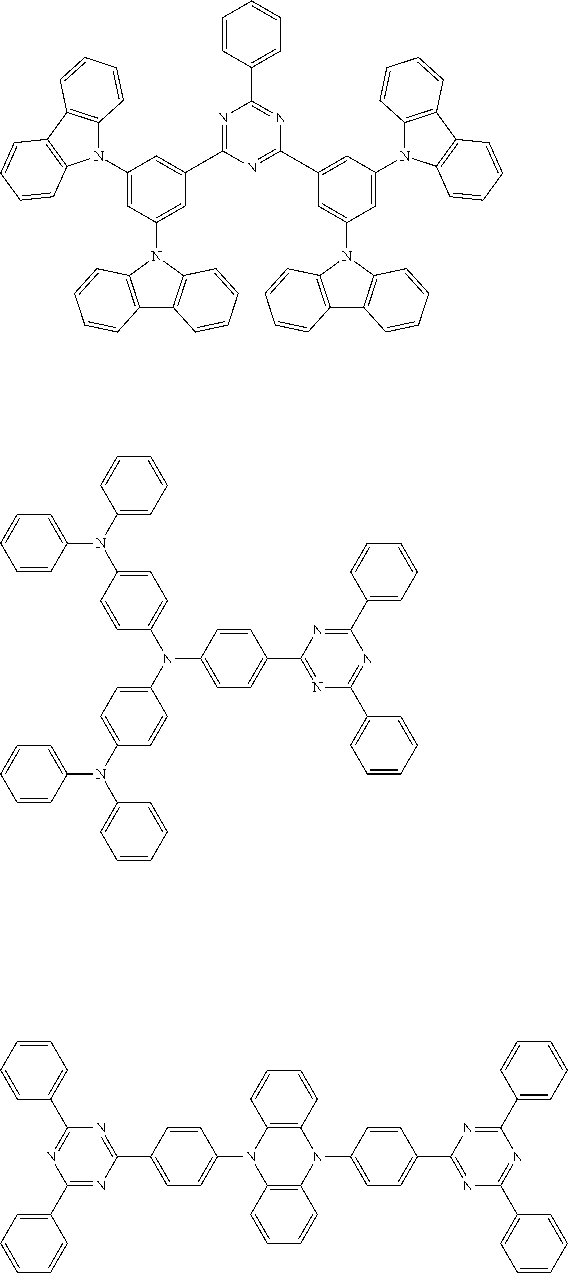 [SMILES: c1ccc(-c2nc(-c3cc(-n4c5ccccc5c5ccccc54)cc(-n4c5ccccc5c5ccccc54)c3)nc(-c3cc(-n4c5ccccc5c5ccccc54)cc(-n4c5ccccc5c5ccccc54)c3)n2)cc1 ; c1ccc(-c2nc(-c3ccccc3)nc(-c3ccc(N(c4ccc(N(c5ccccc5)c5ccccc5)cc4)c4ccc(N(c5ccccc5)c5ccccc5)cc4)cc3)n2)cc1 ; c1ccc(-c2nc(-c3ccccc3)nc(-c3ccc(N4c5ccccc5N(c5ccc(-c6nc(-c7ccccc7)nc(-c7ccccc7)n6)cc5)c5ccccc54)cc3)n2)cc1]

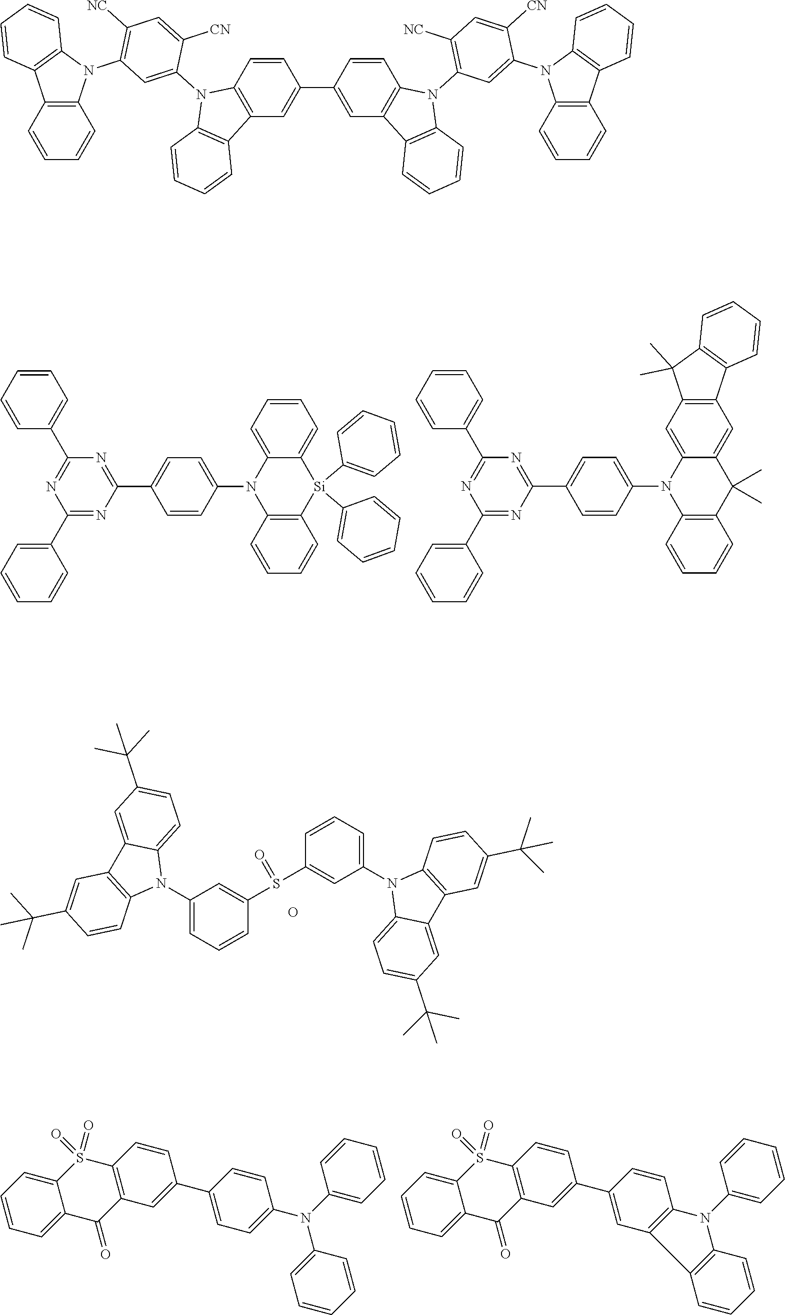

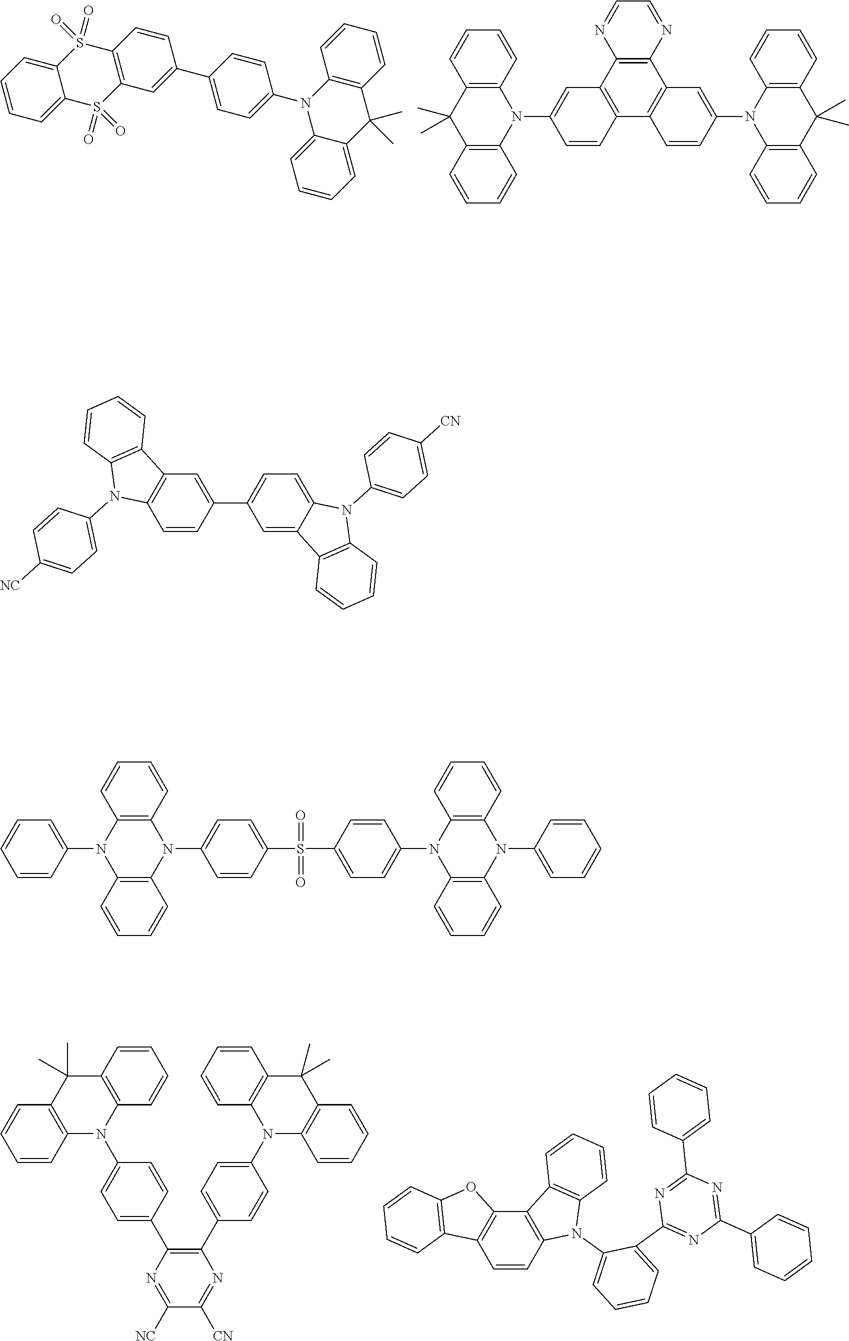

In some embodiments, the organic compound is used in evaporation-based OLED. For this purpose, the molecular weight of the organic compound ≤1000 g/mol, preferably ≤900 g/mol, more preferably ≤850 g/mol, further preferably ≤800 g/mol, and most preferably ≤700 g/mol.

Another object of the present disclosure is to provide a material for the printed OLEDs.

For this purpose, the molecular weight of the organic compound ≥700 g/mol, preferably ≥800 g/mol, more preferably ≥900 g/mol, further preferably ≥1000 g/mol, and most preferably ≥1100 g/mol.

In some embodiments, the organic compound as described herein has a solubility of ≥10 mg/mL in toluene at 25° C., preferably ≥15 mg/mL, and most preferably ≥20 mg/mL.

In yet another aspect, the present disclosure further provides a formulation or a ink comprising an organic compound or a polymer or a mixture as described herein, and at least one organic solvent.

The viscosity and surface tension of the ink are important parameters in printing processes. A suitable ink surface tension is required for the specific substrates and the specific printing methods.

In some embodiments, the surface tension of the ink of the present disclosure at 25° C. is in the range of 19 dyne/cm to 50 dyne/cm; more preferably in the range of 22 dyne/cm to 35 dyne/cm; and most preferably in the range of 25 dyne/cm to 33 dyne/cm.

In some embodiments, the viscosity of the ink of the present disclosure at 25° C. is in the range of from about 1 cps to 100 cps; particularly in the range of 1 cps to 50 cps; more particularly in the range of 1.5 cps to 20 cps; and most particularly in the range of 4.0 cps to 20 cps. The resulting formulation will be particularly suitable for ink-jet printing.

The viscosity can be adjusted by different methods, such as by the selection of appropriate organic solvent and the concentration of the functional materials in the ink. In the ink comprising the above-mentioned metal-organic complexes or polymers as described herein facilitate the adjustment of the printing ink in the appropriate range according to the printing method used. Generally, in the formulation comprising the functional material as described herein, the weight ratio of the functional material ranges from 0.3 wt % to 30 wt %, preferably in the range of 0.5 wt % to 20 wt %, more preferably in the range of 0.5 wt % to 15 wt %, further preferably in the range of 0.5 wt % to 10 wt %, and most preferably in the range of 1 wt % to 5 wt %.

In some embodiments, the at least one organic solvent of the ink as described herein is selected from aromatic-based or heteroaromatic-based solvents, particular in aliphatic chain/ring substituted aromatic solvents, aromatic ketone solvents, or aromatic ether solvents.

Examples of solvents suitable for the present disclosure include, but not limited to aromatic-based or heteroaromatic-based solvents, such as p-diisopropylbenzene, amylbenzene, tetralin, cyclohexylbenzene, chloronaphtalene, 1,4-dimethylnaphthalene, 3-isopropylbenzene, p-methylisopropylbenzene, dipentylbenzene, tripentylbenzene, pentyltoluene, o-xylene, m-xylene, p-xylene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diiisopropylbenzene, 1-methoxynaphthalene, cyclohexylbenzene, dimethylnaphthalene, 3-isopropylbiphenyl, p-methylisopropylbenzene, 1-methylnaphthalene, 1,2,4-trichlorobenzene, 1,3-dipropoxybenzene, 4,4-difluorobenzenemethane, 1,2-dimethoxy-4-(1-propenyl)benzene, diphenylmethane, 2-phenylpyridine, 3-phenylpyridine, N-methyldiphenylamine, 4-isopropylbipheny, α,α-dichlorodiphenylmethane, 4-(3-phenylpropyl)pyridine, benzyl benzoatel, 1,1-bis(3,4-dimethylphenyl) ethane, 2-isopropylnaphthalene, dibenzyl ether, etc; ketone-based solvents, such as 1-tetrahydronaphthalone, 2-tetrahydronaphthalone, 2-(phenylepoxy)tetrahydronaphthalone, 6-(methoxy)tetrahydronaphthalone, acetophenone, phenylacetone, benzophenone, and derivatives thereof such as 4-methyl acetophenone, 3-methyl acetophenone, 2-methyl acetophenone, 4-methyl propanone, 3-methyl propanone, 2-methyl propanone, isophorone, 2,6,8-trimethyl-4-nonanone, fenchone, 2-nonanone, 3-nonanone, 5-nonanone, 2-decanone, 2,5-hexanedione, phoron, di-n-amyl ketone, etc; aromatic ether solvents, such as 3-phenoxytoluene, butoxybenzene, benzylbutylbenzene, p-anisaldehyde dimethyl acetal, tetrahydro-2-phenoxy-2H-pyran, 1,2-dimethoxy-4-(1-propenyl)benzene, 1,4-benzodioxane, 1,3-dipropylbenzene, 2,5-dimethoxytoluene, 4-ethylphenyl ether, 1,2,4-trimethoxybenzene, 4-(1-propenyl)-1,2-dimethoxybenzene, 1,3-dimethoxybenzene, glycidyl phenyl ether, dibenzyl ether, 4-tert-butyl anisole, trans-anethole, 1,2-dimethoxybenzene, 1-methoxynaphthalene, diphenyl ether, 2-phenoxymethyl ether, 2-phenoxytetrahydrofuran, ethyl-2-naphthyl ether, dipentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, etc; ester solvent, such as alkyl octanoate, alkyl sebacate, alkyl stearate, alkyl benzoate, alkyl phenylacetate, alkyl cinnamate, alkyl oxalate, alkyl maleate, alkyl lactone, alkyl oleate, etc.

Further, the at least one organic solvent of the ink as described herein can be selected from aliphatic ketones, such as 2-nonanone, 3-nonanone, 5-nonanone, 2-decanone, 2,5-hexanedione, 2,6,8-trimethyl-4-nonanone, phorone, 6-undecanone, etc; and the at least one organic solvent of the ink can be selected from aliphatic, ethers, such as, dipentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, etc.

In some embodiments, the printing ink further comprises another organic solvent. Examples of the another organic solvent include, but not limited to: methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxytoluene, 1,1,1-trichloroethane, 1,1,2,2-tetrachloroethane, ethyl acetate, butyl acetate, dimethylformamide, dimethylacetamide, dimethylsulfoxide, tetraline, decalin, indene, and/or mixtures thereof.

In some embodiments, the formulation as described herein is a solution.

In some embodiments, the formulation as described herein is a dispersion.

The formulations in the embodiments of the present disclosure may comprise the organic compound or the polymer or the mixture of 0.01 wt % to 20 wt %, preferably 0.1 wt % to 15 wt %, more preferably 0.2 wt % to 10 wt %, and most preferably 0.25 wt % to 5 wt %.

The present disclosure also relates to the use of the formulation as coatings or printing inks in the preparation of organic electronic devices, particularly preferably by printing or coating processing methods.

Where suitable printing or coating techniques include, but not limited to ink-jet printing, nozzle printing, gravure printing, typographic printing, screen printing, dip coating, spin coating, blade coating, roller printing, torsion roll printing, planographic printing, flexographic printing, rotary printing, spray coating, brush coating, pad printing, slit die coating, and so on. Preferred techniques are ink-jet printing, nozzle printing, and gravure printing. The solution or dispersion may additionally comprise one or more components, such as surface active compounds, lubricants, wetting agents, dispersing agents, hydrophobic agents, binders, etc., which are used to adjust the viscosity and film forming properties, or to improve adhesion, etc. For more information on printing technologies and their requirements for solutions, such as solvent, concentration, viscosity, etc, please refer to Handbook of Print Media: Technologies and Production Methods, edited by Helmut Kipphan, ISBN 3-540-67326-1.

The present disclosure further provides an application of the organic compound as described herein in organic electronic devices. The organic electronic device may be selected from, but not limited to, an organic light-emitting diode (OLED), an organic photovoltaic cell (OPV), an organic light emitting electrochemical cell (OLEEC), an organic field effect transistor (OFET), an organic light emitting field effect transistor, an organic laser, an organic spintronic electronic device, an organic sensor, an organic plasmon emitting diode (OPED), etc., particularly preferably is an OLED.

Preferably, the organic compound is used in the light-emitting layer of the OLED.

In yet another aspect, the present disclosure further provides an organic electronic device comprising an organic compound or a polymer or a mixture or a formulation as described herein; preferably, the organic electronic device comprises at least one functional layer, where at least one of the functional layer comprises an organic compound or a polymer or a mixture or a formulation as described herein. Further, the organic electronic device comprises a cathode, an anode and at least one functional layer, where at least of the functional layer comprises an organic compound or a polymer or a mixture as described herein or is formed by using the formulation as described herein. At least one of the functional layer is selected from a hole-injection layer (HIL), a hole-transport layer (HTL), a light-emitting layer (EML), an electron-blocking layer (EBL), an electron-injection layer (EIL), an electron-transport layer (ETL), a hole-blocking layer (HBL), or a charge generation layer (CGL).

The organic electronic device may be selected from, but not limited to, an organic light-emitting diode (OLED), an organic photovoltaic cell (OPV), an organic light emitting electrochemical cell (OLEEC), an organic field effect transistor (OFET), an organic light emitting field effect transistor, an organic laser, an organic spintronic device, an organic sensor, or an organic plasmon emitting diode (OPED), etc., particularly preferably is an organic electroluminescent device, such as an OLED, an OLEEC, an organic light emitting field effect transistor.

Preferably, the organic electronic device as described herein is an electroluminescent device, which comprises a substrate, an anode, at least one light-emitting layer, a cathode, and further optionally comprises a hole-transport layer. In some embodiments, the hole-transport layer comprises an organic compound or a polymer or a mixture as described herein.

In some embodiments, the organic electronic device comprises a light-emitting layer, where the light-emitting layer comprises an organic compound or a polymer or a mixture as described herein, more preferably, the light-emitting layer comprises an organic compound or a polymer or a mixture as described herein, and at least one emitting material, which is preferably selected from a fluorescent emitting material, a phosphorescent emitting material, or a TADF material.

The device structure of the electroluminescent device is described below, but not limited to.

The substrate should be opaque or transparent. A transparent substrate could be used to produce a transparent light-emitting device (for example: Bulovic et al., Nature, 1996, 380, p29, and Gu et al., Appl. Phys. Lett., 1996, 68, p2606). The substrate can be rigid or flexible, e.g. it can be plastic, metal, semiconductor wafer, or glass. Preferably, the substrate has a smooth surface. Particularly ideal are substrates without surface defects. In some embodiments, the substrate is flexible and can be selected from a polymer film or plastic with a glass transition temperature (Tg)>150° C., preferably >200° C., more preferably >250° C., and most preferably >300° C. Examples of the suitable flexible substrates include poly ethylene terephthalate (PET) and polyethylene glycol (2,6-naphthalene) (PEN).

The anode may be a conductive metal, or a metal oxide, or a conductive polymer. The anode should be able to easily inject holes into a hole-injection layer (HIL), a hole-transport layer (HTL), or a light-emitting layer. In some embodiments, the absolute value of the difference between the work function of the anode and the HOMO energy level/valence band energy level of the emitter of the light-emitting layer or the p-type semiconductor materials of the hole-injection layer (HIL)/hole-transport layer (HTL)/electron-blocking layer (EBL)<0.5 eV, preferably <0.3 eV, and most preferably <0.2 eV. Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by the general technicians in this field. The anode materials can be deposited using any suitable technique, such as a suitable physical vapor deposition method including RF magnetron sputtering, vacuum thermal evaporation, e-beam, etc. In some embodiments, the anode is patterned. Patterned conductive ITO substrates are commercially available and can be used to produce the devices as described herein.

The cathode may be a conductive metal or a metal oxide. The cathode should be able to easily inject electrons into the electron-injection layer (EIL), the electron-transport layer (ETL), or the directly into the light-emitting layer. In some embodiments, the absolute value of the difference between the work function of the cathode and the LUMO energy level/conduction band energy level of the emitter of the light-emitting layer, or the n-type semiconductor materials of the electron-injection layer (EIL)/electron-transport layer (ETL)/hole-blocking layer (HBL) <0.5 eV, preferably <0.3 eV, and most preferably <0.2 eV. In principle, all materials those can be used as cathodes for OLEDs may be applied as cathode materials for the devices as described herein. Examples of cathode materials include, but not limited to: Al, Au, Ag, Ca, Ba, Mg, LiF/Al, MgAg alloy, BaF₂/Al, Cu, Fe, Co, Ni, Mn, Pd, Pt, ITO, etc. The cathode materials can be deposited using any suitable technique, such as the suitable physical vapor deposition method including RF magnetron sputtering, vacuum thermal evaporation, e-beam, etc.

The OLED may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in detail above.

In some embodiments, the electron-transport layer (ETL) or hole-blocking layer (HBL) of the light emitting device as described herein comprises an organic compound or a polymer or a mixture as described herein, and is formed by using the solution processing method.

The emitting wavelength of the light emitting device is between 300 nm and 1000 nm, preferably between 350 nm and 900 nm, more preferably between 400 nm and 800 nm.

The present disclosure further provides the applications of electroluminescent devices in various electronic equipment, including, but not limited to, display devices, lighting equipment, light sources, sensors, etc.

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### Specific Embodiment

1. The synthetic methods of the organic compound are exemplified, but the present disclosure is not limited to the following embodiments.

Preparation of intermediate 1e:

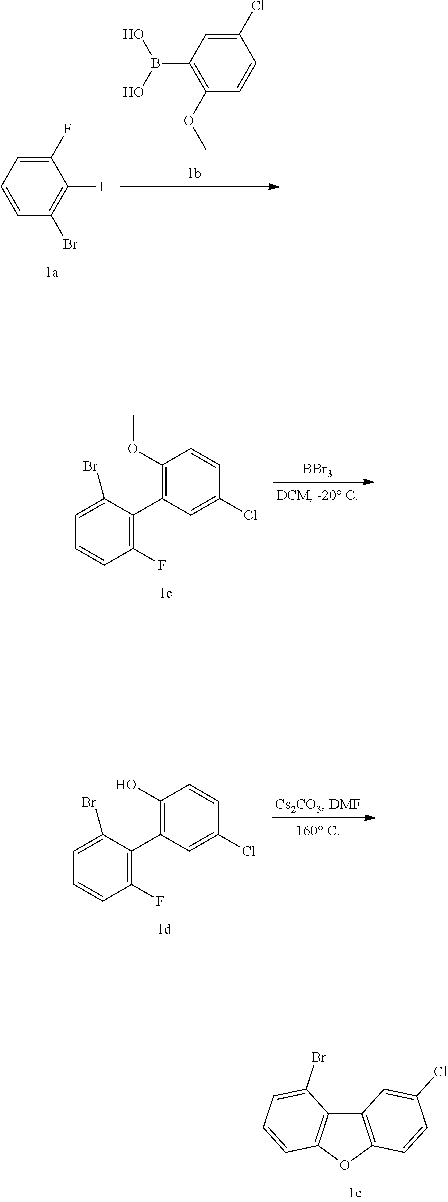 [SMILES: Fc1cccc(Br)c1I ; COc1ccc(Cl)cc1B(O)O ; COc1ccc(Cl)cc1-c1c(F)cccc1Br ; "BBr3" ; Oc1ccc(Cl)cc1-c1c(F)cccc1Br ; Clc1ccc2oc3cccc(Br)c3c2c1]

1a (30 g), 1b (30 g), potassium carbonate (25 g), tetrakis (triphenylphosphine) palladium (1 g), and 300 mL of toluene/50 mL of ethanol/50 mL of water was added to a three-necked flask under N₂ atmosphere. After heating up to 100° C., the resulting mixture was refluxed for 12 h. After the mixture was cooled, the resulting solution was extracted with ethyl acetate and deionized water, the organic phase was washed with water two times, dried, filtered, and the solvent was removed to obtain 28 g of the crude product (intermediate 1c). After that, the resulting sample was purified by column chromatography (petroleum ether: ethyl acetate=15:1) to yield 25.7 g (87% yield) of intermediate 1c. The molecular ion mass was determined to be 315.6 (calculated value: 315.57) by mass spectrometry.

Intermediate 1c (25.7 g) and dichloromethane (200 mL) were added to a dry three-necked flask, then the resulting mixture was cooled down to below 20° C. After adding 50 mL of boron tribromide dropwise to the reaction system, the resulting mixture was naturally heated to reflux for 12 h. After the reaction was completed, the resulting mixture was quenched with ethanol in an ice bath, and extracted with dichloromethane and deionized water. After washing with deionized water two times, the result was dried, filtered, and the solvent was removed to obtain 20 g of the crude product (1d) which was used directly in the next step of the reaction without any other treatment.

1d (20 g) was added to a single-necked flask, then N,N-dimethylformamide (300 mL) and cesium carbonate (25 g) were added. After heating up to 140° C., the resulting mixture was melted, stirred, and reacted for 12 h. After the mixture was cooled, the resulting mixture was extracted with deionized water and ethyl acetate, the organic phase was washed with water three times, dried to obtain 18.4 g of the crude product (intermediate 1e). After that, the resulting sample was purified by column chromatography (petroleum ether: ethyl acetate=15:1) to yield 16 g (82% yield) of intermediate 1e. The molecular ion mass was determined to be 281.5 (calculated value: 281.53) by mass spectrometry.

Preparation of intermediate 1m:

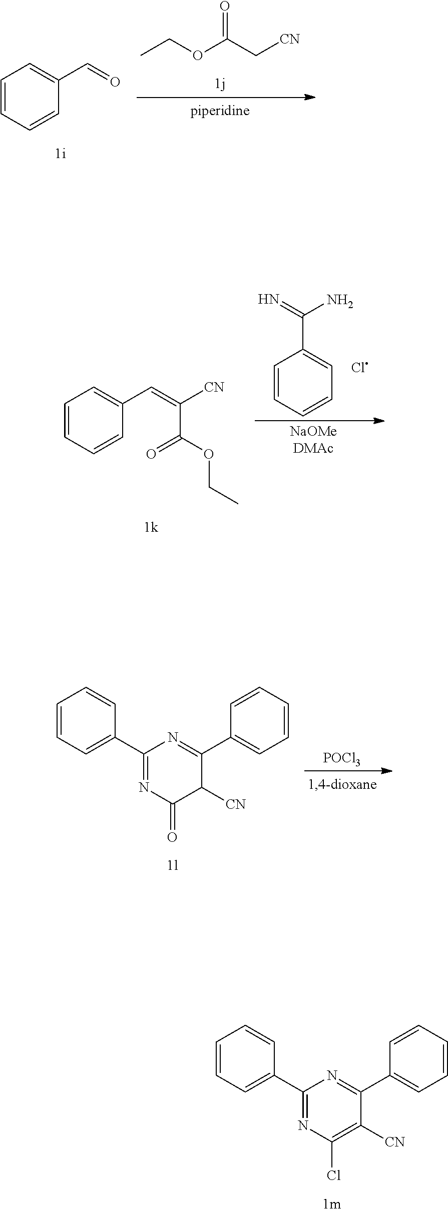

1i (30 g), 1j (25 g), piperidine (300 mL) were added to a three-necked flask under N₂ atmosphere. After heating up to 100° C., the resulting mixture was refluxed for 12 h. After the mixture was cooled, the resulting sample was purified by column chromatography (petroleum ether: ethyl acetate=20:1) to yield 20.8 g (60% yield) of 1k. The molecular ion mass was determined to be 201.2 (calculated value: 201.23) by mass spectrometry.

1k (20.8 g), benzamidine hydrochloride (25 g), sodium methanol (25 g), and DMAc (300 mL) were added to a three-necked flask under N₂ atmosphere. After heating up to 100° C., the resulting mixture was refluxed for 12 h. After the mixture was cooled, the resulting sample was purified by column chromatography (petroleum ether: ethyl acetate=20:1) to yield 15.7 g (58% yield) of 1l. The molecular ion mass was determined to be 272.3 (calculated value: 272.29) by mass spectrometry.

1l (15.7 g), phosphorus oxychloride (20 g), and 1,4-dioxane (300 mL) were added to a three-necked flask under N₂ atmosphere. After heating up to 100° C., the resulting mixture was refluxed for 12 h. After the mixture was cooled, 1000 mL of water was added to precipitate the product, filtered, and washed with ethanol to yield 10.2 g (56% yield) of intermediate 1m. The molecular ion mass was determined to be 291.7 (calculated value: 291.74) by mass spectrometry.

Preparation of intermediate 3d:

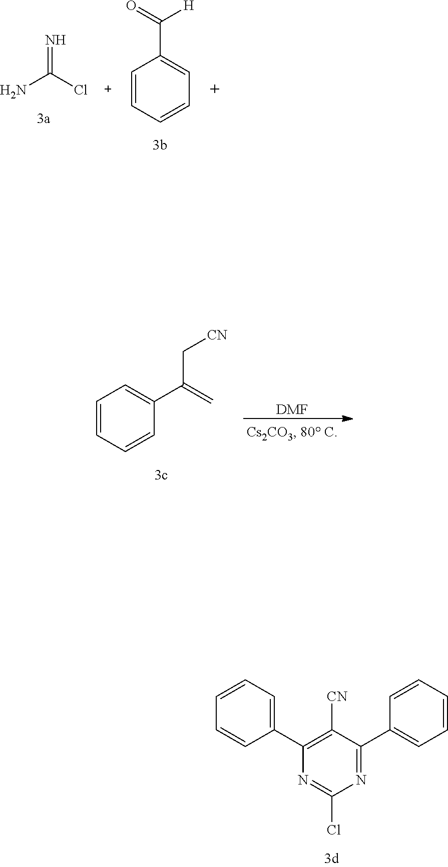

3a (20 g), 3b (25 g), 3c (27 g), cesium carbonate (30 g), and DMF (1000 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 23.2 g (90% yield) of intermediate 3d. The molecular ion mass was determined to be 291.7 (calculated value: 291.74) by mass spectrometry.

Preparation of compound 1:

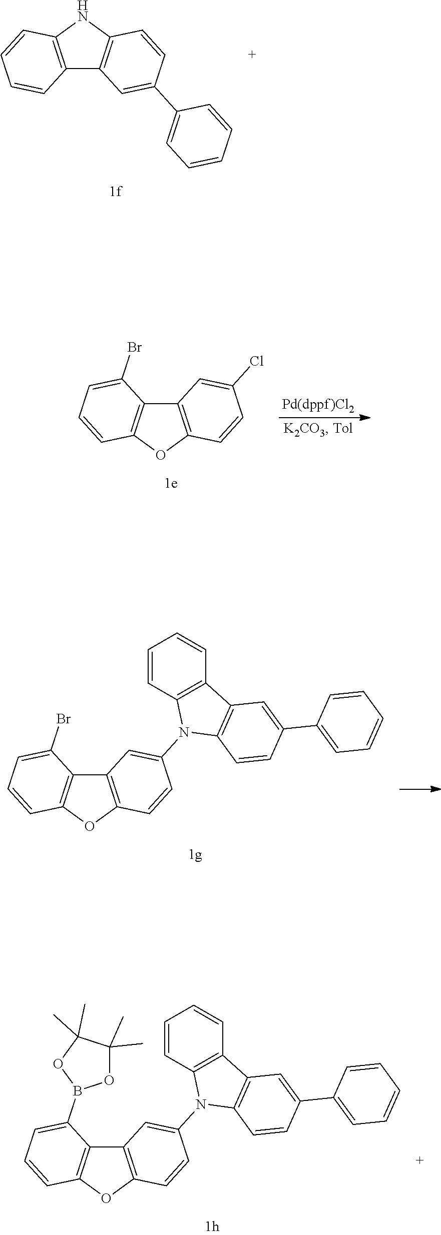 [SMILES: c1ccc(-c2ccc3[nH]c4ccccc4c3c2)cc1 ; Clc1ccc2oc3cccc(Br)c3c2c1 ; "K2CO3, Tol" ; Brc1cccc2oc3ccc(-n4c5ccccc5c5cc(-c6ccccc6)ccc54)cc3c12 ; CC1(C)OB(c2cccc3oc4ccc(-n5c6ccccc6c6cc(-c7ccccc7)ccc65)cc4c23)OC1(C)C]

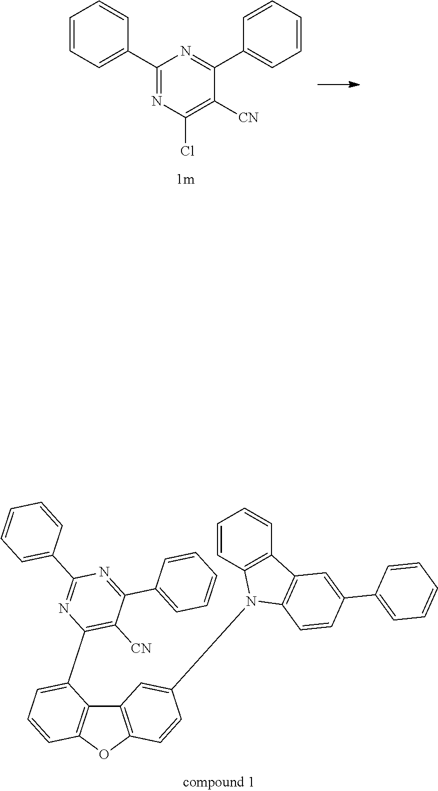 [SMILES: N#Cc1c(Cl)nc(-c2ccccc2)nc1-c1ccccc1 ; N#Cc1c(-c2ccccc2)nc(-c2ccccc2)nc1-c1cccc2oc3ccc(-n4c5ccccc5c5cc(-c6ccccc6)ccc54)cc3c12]

1f (20 g), intermediate 1e (15 g), potassium carbonate (15 g), catalyst Pd(dppf)Cl₂ (1 g), and toluene (300 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 18 g (86% yield) of 1g. The molecular ion mass was determined to be 488.4 (calculated value: 488.38) by mass spectrometry.

1g (18 g), bis(pinacolato)diboron (20 g), potassium acetate (15 g), catalyst Pd(dppf)Cl₂ (1 g), and toluene (300 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 19.5 g (89% yield) of 1h. The molecular ion mass was determined to be 535.5 (calculated value: 535.45) by mass spectrometry.

Intermediate 1m (10 g), 1h (10 g), potassium acetate (10 g), catalyst Pd(dppf)Cl₂ (1 g), toluene (200 mL), ethanol (40 mL), and water (40 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 12.3 g (89% yield) of compound 1. The molecular ion mass was determined to be 664.7 (calculated value: 664.77) by mass spectrometry.

Preparation of compound 2:

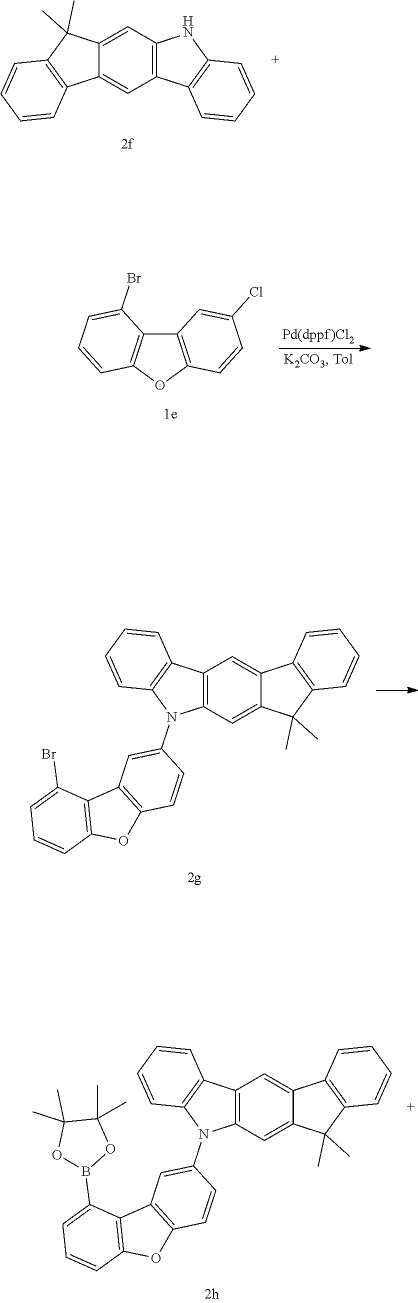 [SMILES: CC1(C)c2ccccc2-c2cc3c(cc21)[nH]c1ccccc13 ; Clc1ccc2oc3cccc(Br)c3c2c1 ; "K2CO3, Tol" ; CC1(C)c2ccccc2-c2cc3c4ccccc4n(-c4ccc5oc6cccc(Br)c6c5c4)c3cc21 ; CC1(C)c2ccccc2-c2cc3c4ccccc4n(-c4ccc5oc6cccc(B7OC(C)(C)C(C)(C)O7)c6c5c4)c3cc21]

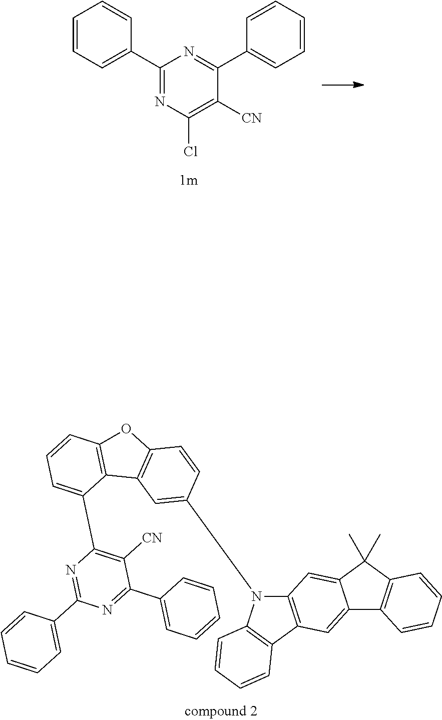 [SMILES: N#Cc1c(Cl)nc(-c2ccccc2)nc1-c1ccccc1 ; CC1(C)c2ccccc2-c2cc3c4ccccc4n(-c4ccc5oc6cccc(-c7nc(-c8ccccc8)nc(-c8ccccc8)c7C#N)c6c5c4)c3cc21]

2f (20 g), intermediate 1e (15 g), potassium carbonate (15 g), catalyst Pd(dppf)Cl₂ (1 g), and toluene (300 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 19 g (85% yield) of 2g. The molecular ion mass was determined to be 528.4 (calculated value: 528.45) by mass spectrometry.

2g (19 g), bis(pinacolato)diboron (20 g), potassium acetate (15 g), catalyst Pd(dppf)Cl₂ (1 g), and toluene (300 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 22 g (92% yield) of 2h. The molecular ion mass was determined to be 575.5 (calculated value: 575.52) by mass spectrometry.

Intermediate 1m (10 g), 2h (10 g), potassium acetate (10 g), catalyst Pd(dppf)Cl₂ (1 g), toluene (200 mL), ethanol (40 mL), and water (40 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 12.2 g (85% yield) of compound 2. The molecular ion mass was determined to be 704.8 (calculated value: 704.83) by mass spectrometry.

Preparation of compound 3:

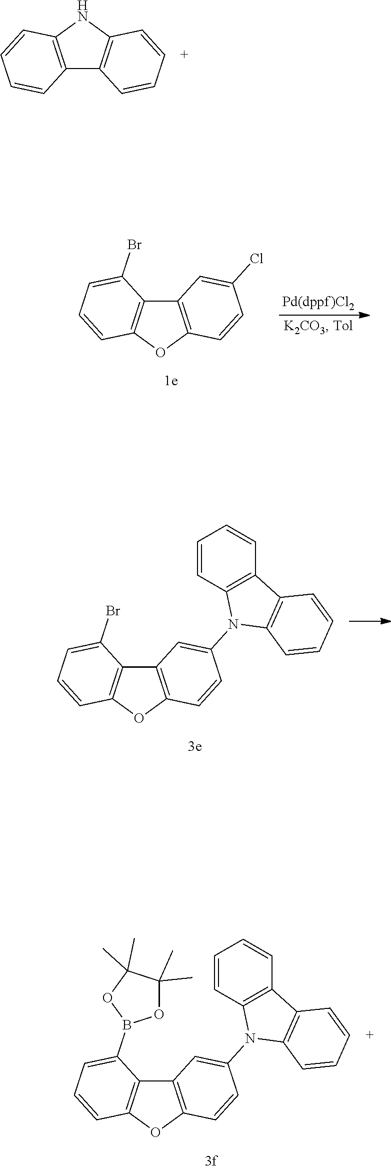 [SMILES: c1ccc2c(c1)[nH]c1ccccc12 ; Clc1ccc2oc3cccc(Br)c3c2c1 ; "K2CO3, Tol" ; Brc1cccc2oc3ccc(-n4c5ccccc5c5ccccc54)cc3c12 ; CC1(C)OB(c2cccc3oc4ccc(-n5c6ccccc6c6ccccc65)cc4c23)OC1(C)C]

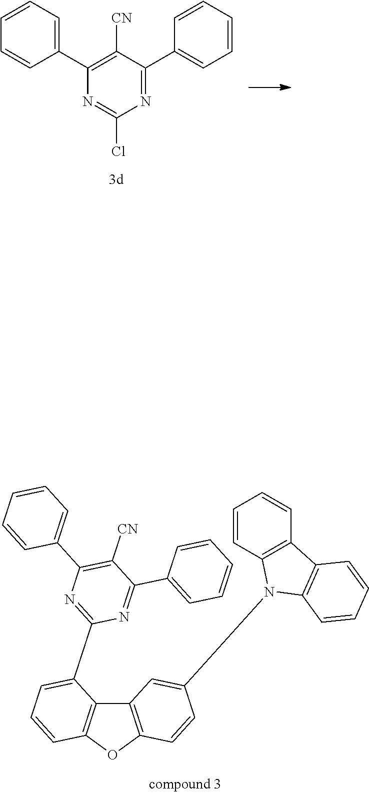 [SMILES: N#Cc1c(-c2ccccc2)nc(Cl)nc1-c1ccccc1 ; N#Cc1c(-c2ccccc2)nc(-c2cccc3oc4ccc(-n5c6ccccc6c6ccccc65)cc4c23)nc1-c1ccccc1]

Carbazole (20 g), intermediate 1e (15 g), potassium carbonate (15 g), catalyst Pd(dppf)Cl₂ (1 g), and toluene (300 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 19 g (85% yield) of 3e. The molecular ion mass was determined to be 412.3 (calculated value: 412.29) by mass spectrometry.

3e (19 g), bis(pinacolato)diboron (20 g), potassium acetate (15 g), catalyst Pd(dppf)Cl₂ (1 g), and toluene (300 mL) were added to a dry three-necked flask under N₂ atmosphere. After heating up to 100°° C., the resulting mixture was stirred and refluxed for 24 h. After the reaction was completed, the resulting sample was purified by column chromatography (petroleum ether: ethyl acetate=25:1) to yield 22 g (92% yield) of 3f. The molecular ion mass was determined to be 459.3 (calculated value: 459.35) by mass spectrometry.

Intermediate 3d (10 g), 3f (10 g), potassium acetate (10 g), catalyst Pd(dppf)Cl₂ (1 g), toluene (200 mL), ethanol (40 mL), and water (40 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 12.1 g (84% yield) of compound 3. The molecular ion mass was determined to be 588.6 (calculated value: 588.67) by mass spectrometry.

Preparation of compound 4:

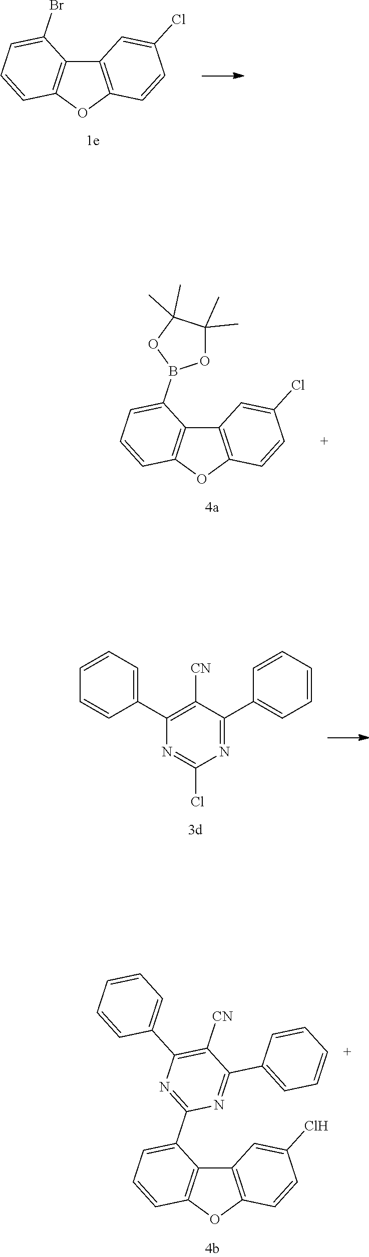 [SMILES: Clc1ccc2oc3cccc(Br)c3c2c1 ; CC1(C)OB(c2cccc3oc4ccc(Cl)cc4c23)OC1(C)C ; N#Cc1c(-c2ccccc2)nc(Cl)nc1-c1ccccc1 ; N#Cc1c(-c2ccccc2)nc(-c2cccc3oc4ccc(Cl)cc4c23)nc1-c1ccccc1]

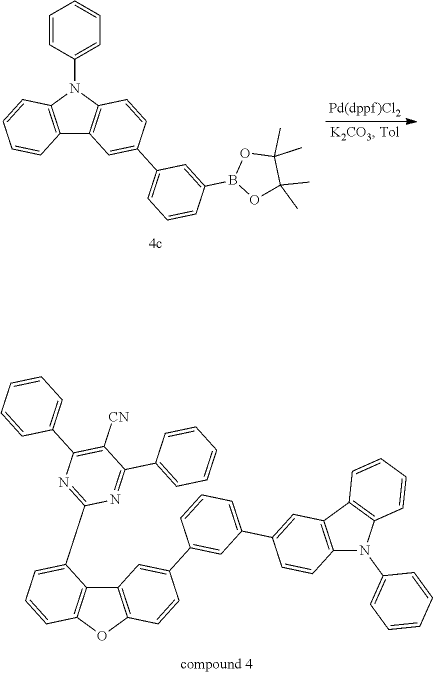

Intermediate 1e (15 g), bis(pinacolato)diboron (20 g), potassium acetate (15 g), catalyst Pd(dppf)Cl₂ (1 g), and toluene (300 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 17 g (95% yield) of 4a. The molecular ion mass was determined to be 328.6 (calculated value: 328.60) by mass spectrometry.

Intermediate 3d (15 g), 4a (15 g), potassium carbonate (20 g), tetrakis (triphenylphosphine) palladium (1 g), and 300 mL of toluene/50 mL of ethanol/50 mL of water were added to a three-necked flask under N₂ atmosphere. After heating up to 100° C., the resulting mixture was refluxed for 12 h. After the mixture was cooled, the resulting solution was extracted with ethyl acetate and deionized water, the organic phase was washed with water two times, dried, filtered, and the solvent was removed to obtain 18 g of the crude product (4b). After that, the resulting sample was purified by column chromatography (petroleum ether: ethyl acetate=15:1) to yield 16 g (62% yield) of 4b. The molecular ion mass was determined to be 457.9 (calculated value: 457.92) by mass spectrometry.

4b (10 g), 4c (10 g), potassium carbonate (20 g), tetrakis (triphenylphosphine) palladium (1 g), and 300 mL of toluene/50 mL of ethanol/50 mL of water were added to a three-necked flask under N₂ atmosphere. After heating up to 100° C., the resulting mixture was refluxed for 12 h. After the mixture was cooled, the resulting solution was extracted with ethyl acetate and deionized water, the organic phase was washed with water two times, dried, filtered, and the solvent was removed. After that, the resulting sample was purified by column chromatography (petroleum ether: ethyl acetate=15:1) to yield 11 g (65% yield) of compound 4. The molecular ion mass was determined to be 740.8 (calculated value: 740.87) by mass spectrometry.

Preparation of compound 5:

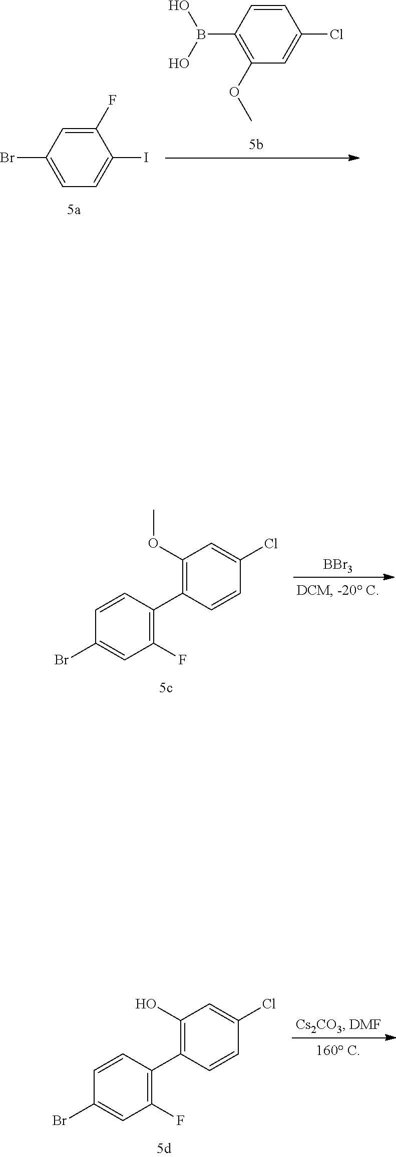

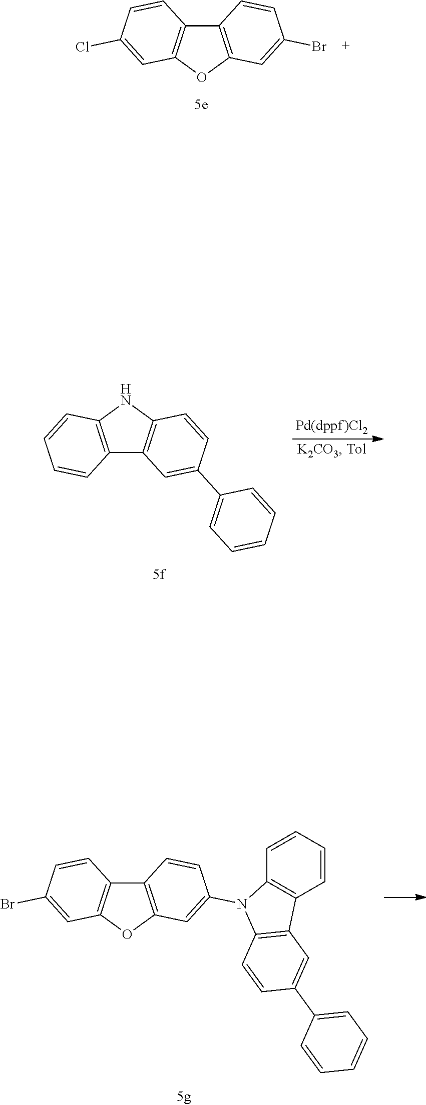 [SMILES: Clc1ccc2c(c1)oc1cc(Br)ccc12 ; c1ccc(-c2ccc3[nH]c4ccccc4c3c2)cc1 ; "K2CO3, Tol" ; Brc1ccc2c(c1)oc1cc(-n3c4ccccc4c4cc(-c5ccccc5)ccc43)ccc12]

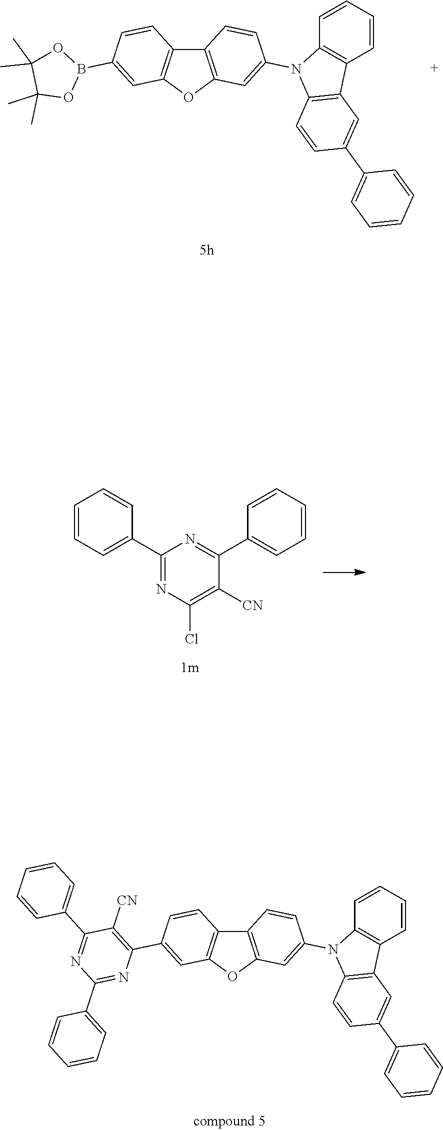 [SMILES: CC1(C)OB(c2ccc3c(c2)oc2cc(-n4c5ccccc5c5cc(-c6ccccc6)ccc54)ccc23)OC1(C)C ; N#Cc1c(Cl)nc(-c2ccccc2)nc1-c1ccccc1 ; N#Cc1c(-c2ccccc2)nc(-c2ccccc2)nc1-c1ccc2c(c1)oc1cc(-n3c4ccccc4c4cc(-c5ccccc5)ccc43)ccc12]

5a (30 g), 5b (30 g), potassium carbonate (25 g), tetrakis (triphenylphosphine) palladium (1 g), and 300 mL of toluene/50 mL of ethanol/50 mL of water were added to a three-necked flask under N₂ atmosphere. After heating up to 100° C., the resulting mixture was refluxed for 12 h. After the mixture was cooled, the resulting solution was extracted with ethyl acetate and deionized water, the organic phase was washed with water two times, dried, filtered, and the solvent was removed to obtain 28 g of the crude product (intermediate 5c). After that, the resulting sample was purified by column chromatography (petroleum ether: ethyl acetate=15:1) to yield 28.2 g (92% yield) of intermediate 5c. The molecular ion mass was determined to be 315.6 (calculated value: 315.57) by mass spectrometry.

Intermediate 5c (25 g) and dichloromethane (200 mL) were added to a dry three-necked flask, then the resulting mixture was cooled down to below 20° C. After adding 50 mL of boron tribromide dropwise to the reaction system, the resulting mixture was naturally heated to reflux for 12 h. After the reaction was completed, the resulting mixture was quenched with ethanol in an ice bath, and extracted with dichloromethane and deionized water. After washing with deionized water two times, the result was dried, filtered, and the solvent was removed to obtain 20 g of the crude product (5d) which was used directly in the next step of the reaction without any other treatment.

5d (20 g) was added to a single-necked flask, then N,N-dimethylformamide (300 mL) and cesium carbonate (25 g) were added. After heating up to 140° C., the resulting mixture was melted, stirred, and reacted for 12 h. After the mixture was cooled, the resulting mixture was extracted with deionized water and ethyl acetate, the organic phase was washed with water three times, dried to obtain 17.5 g of the crude product (5e). After that, the resulting sample was purified by column chromatography (petroleum ether: ethyl acetate=15:1) to yield 15 g (77% yield) of 5e. The molecular ion mass was determined to be 281.5 (calculated value: 281.53) by mass spectrometry.

5f (20 g), 5e (15 g), potassium carbonate (15 g), catalyst Pd(dppf)Cl₂ (1 g), and toluene (300 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 17 g (80% yield) of 5g. The molecular ion mass was determined to be 488.4 (calculated value: 488.38) by mass spectrometry.

5g (15 g), bis(pinacolato)diboron (20 g), potassium acetate (15 g), catalyst Pd(dppf)Cl₂ (1 g), and toluene (300 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 18 g (74% yield) of 5h. The molecular ion mass was determined to be 535.4 (calculated value: 535.45) by mass spectrometry.

Intermediate 1m (10 g), 5h (10 g), potassium acetate (10 g), catalyst Pd(dppf)Cl₂ (1 g), toluene (200 mL), ethanol (40 mL), and water (40 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 13.4 g (86% yield) of compound 5. The molecular ion mass was determined to be 664.7 (calculated value: 664.77) by mass spectrometry.

Preparation of compound 6:

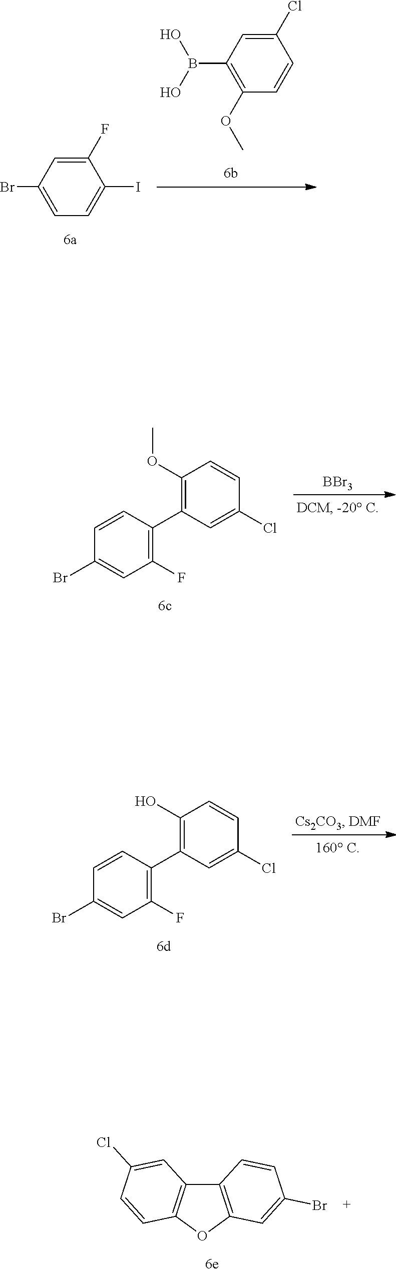 [SMILES: Fc1cc(Br)ccc1I ; COc1ccc(Cl)cc1B(O)O ; COc1ccc(Cl)cc1-c1ccc(Br)cc1F ; "BBr3" ; Oc1ccc(Cl)cc1-c1ccc(Br)cc1F ; Clc1ccc2oc3cc(Br)ccc3c2c1]

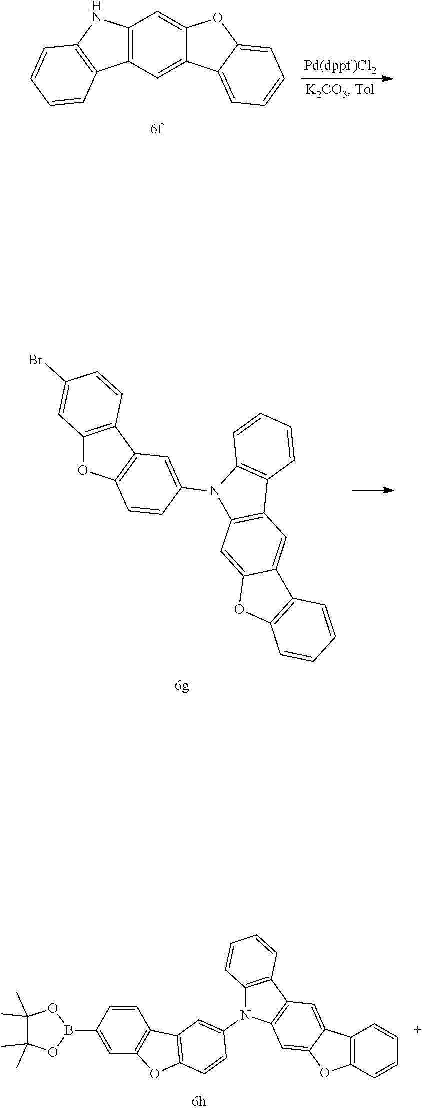

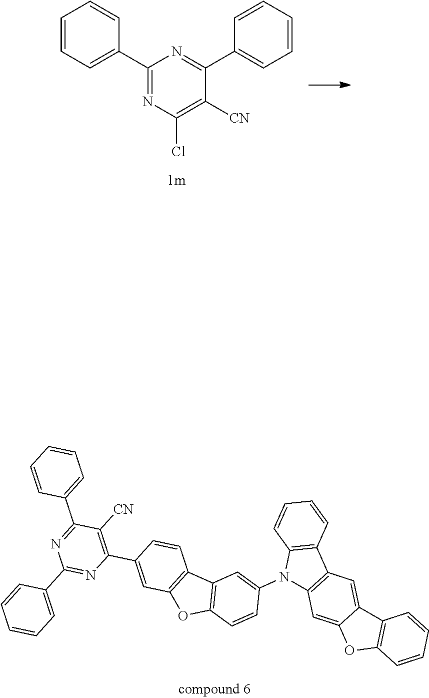 [SMILES: N#Cc1c(Cl)nc(-c2ccccc2)nc1-c1ccccc1 ; N#Cc1c(-c2ccccc2)nc(-c2ccccc2)nc1-c1ccc2c(c1)oc1ccc(-n3c4ccccc4c4cc5c(cc43)oc3ccccc35)cc12]

6a (30 g), 6b (30 g), potassium carbonate (25 g), tetrakis(triphenylphosphine)palladium (1 g), and 300 mL of toluene/50 mL of ethanol/50 mL of water were added to a three-necked flask under N₂ atmosphere. After heating up to 100° C., the resulting mixture was refluxed for 12 h. After the mixture was cooled, the resulting solution was extracted with ethyl acetate and deionized water, the organic phase was washed with water two times, dried, filtered, and the solvent was removed to obtain 28 g of the crude product (intermediate 6c). After that, the resulting sample was purified by column chromatography (petroleum ether: ethyl acetate=15:1) to yield 27.9 g (90% yield) of intermediate 6c. The molecular ion mass was determined to be 315.6 (calculated value: 315.57) by mass spectrometry.

Intermediate 6c (27.9 g) and dichloromethane (200 mL) were added to a dry three-necked flask, then the resulting mixture was cooled down to below 20° C. After adding 50 mL of boron tribromide dropwise to the reaction system, the resulting mixture was naturally heated to reflux for 12 h. After the reaction was completed, the resulting mixture was quenched with ethanol in an ice bath, and extracted with dichloromethane and deionized water. After washing with deionized water two times, the result was dried, filtered, and the solvent was removed to obtain 25 g of the crude product (6d) which was used directly in the next step of the reaction without any other treatment.

6d (25 g) was added to a single-necked flask, then N,N-dimethylformamide (300 mL) and cesium carbonate (25 g) were added. After heating up to 140° C., the resulting mixture was melted, stirred, and reacted for 12 h. After the mixture was cooled, the resulting mixture was extracted with deionized water and ethyl acetate, the organic phase was washed with water three times, dried to obtain 24.3 g of the crude product (6e). After that, the resulting sample was purified by column chromatography (petroleum ether: ethyl acetate=15:1) to yield 20 g (85% yield) of 6e. The molecular ion mass was determined to be 281.5 (calculated value: 281.53) by mass spectrometry.

6f (20 g), 6e (20 g), potassium carbonate (15 g), catalyst Pd(dppf)Cl₂ (1 g), and toluene (300 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 22 g (83% yield) of 6g. The molecular ion mass was determined to be 502.4 (calculated value: 502.37) by mass spectrometry.

6g (22 g), bis(pinacolato)diboron (25 g), potassium acetate (15 g), catalyst Pd(dppf)Cl₂ (1 g), and toluene (300 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 20 g (83% yield) of 6h. The molecular ion mass was determined to be 549.4 (calculated value: 549.43) by mass spectrometry.

Intermediate 1m (10 g), 6h (10 g), potassium acetate (10 g), catalyst Pd(dppf)Cl₂ (1 g), toluene (200 mL), ethanol (40 mL), and water (40 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 13.4 g (86% yield) of compound 6. The molecular ion mass was determined to be 678.7 (calculated value: 678.75) by mass spectrometry.

Preparation of compound 7:

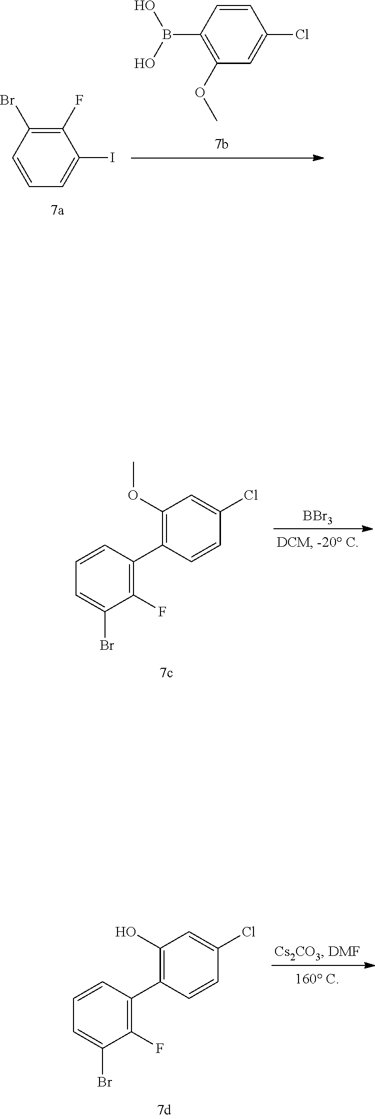

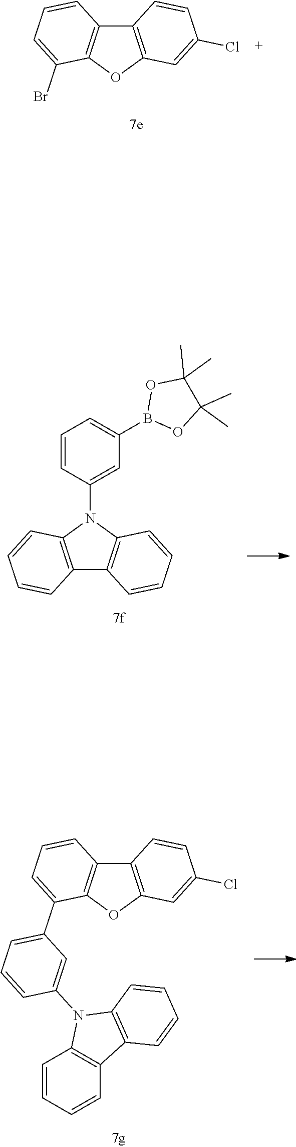 [SMILES: Clc1ccc2c(c1)oc1c(Br)cccc12 ; CC1(C)OB(c2cccc(-n3c4ccccc4c4ccccc43)c2)OC1(C)C ; Clc1ccc2c(c1)oc1c(-c3cccc(-n4c5ccccc5c5ccccc54)c3)cccc12]

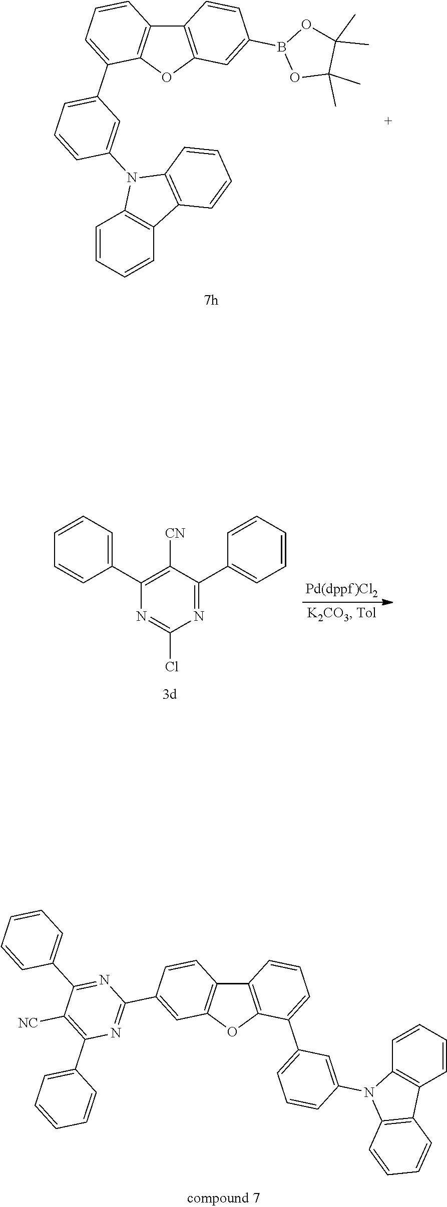 [SMILES: CC1(C)OB(c2ccc3c(c2)oc2c(-c4cccc(-n5c6ccccc6c6ccccc65)c4)cccc23)OC1(C)C ; N#Cc1c(-c2ccccc2)nc(Cl)nc1-c1ccccc1 ; "K2CO3, Tol" ; N#Cc1c(-c2ccccc2)nc(-c2ccc3c(c2)oc2c(-c4cccc(-n5c6ccccc6c6ccccc65)c4)cccc23)nc1-c1ccccc1]

7a (30 g), 7b (30 g), potassium carbonate (25 g), tetrakis(triphenylphosphine)palladium (1 g), and 300 mL of toluene/50 mL of ethanol/50 mL of water were added to a three-necked flask under N₂ atmosphere. After heating up to 100° C., the resulting mixture was refluxed for 12 h. After the mixture was cooled, the resulting solution was extracted with ethyl acetate and deionized water, the organic phase was washed with water two times, dried, filtered, and the solvent was removed to obtain 25.1 g of the crude product (intermediate 7c). After that, the resulting sample was purified by column chromatography (petroleum ether: ethyl acetate=15:1) to yield 24.2 g (81% yield) of intermediate 7c. The molecular ion mass was determined to be 315.6 (calculated value: 315.57) by mass spectrometry.

Intermediate 7c (24.2 g) and dichloromethane (200 mL) were added to a dry three-necked flask, then the resulting mixture was cooled down to below 20° C. After adding 50 mL of boron tribromide dropwise to the reaction system, the resulting mixture was naturally heated to reflux for 12 h. After the reaction was completed, the resulting mixture was quenched with ethanol in an ice bath, and extracted with dichloromethane and deionized water. After washing with deionized water two times, the result was dried, filtered, and the solvent was removed to obtain 23.6 g of the crude product (7d) which was used directly in the next step of the reaction without any other treatment.

7d (23.6 g) was added to a single-necked flask, then N,N-dimethylformamide (300 mL) and cesium carbonate (25 g) were added. After heating up to 140° C., the resulting mixture was melted, stirred, and reacted for 12 h. After the mixture was cooled, the resulting mixture was extracted with deionized water and ethyl acetate, the organic phase was washed with water three times, dried to obtain 22.9 g of the crude product (7e). After that, the resulting sample was purified by column chromatography (petroleum ether: ethyl acetate=15:1) to yield 21.4 g (79% yield) of 7e. The molecular ion mass was determined to be 281.5 (calculated value: 281.53) by mass spectrometry.

7f (25 g), 7e (22.9 g), potassium carbonate (25 g), tetrakis(triphenylphosphine)palladium (1 g) and 300 mL of toluene/50 mL of ethanol/50 mL of water were added to a three-necked flask under N₂ atmosphere. After heating up to 100° C., the resulting mixture was refluxed for 12 h. After the mixture was cooled, the resulting solution was extracted with ethyl acetate and deionized water, the organic phase was washed with water two times, dried, filtered, and the solvent was removed to obtain 24.8 g of the crude product (7g). After that, the resulting sample was purified by column chromatography (petroleum ether: ethyl acetate=15:1) to yield 23.6 g (78% yield) of 7g. The molecular ion mass was determined to be 433.9 (calculated value: 433.93) by mass spectrometry.

7g (23.6 g), bis(pinacolato)diboron (25 g), potassium acetate (15 g), catalyst Pd(dppf)Cl₂ (1 g), and toluene (300 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 20 g (81% yield) of 7h. The molecular ion mass was determined to be 535.4 (calculated value: 535.45) by mass spectrometry.

Intermediate 3d (10 g), 7h (10 g), potassium acetate (10 g), catalyst Pd(dppf)Cl₂ (1 g), toluene (200 mL), ethanol (40 mL), and water (40 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 12.3 g (84% yield) of compound 7. The molecular ion mass was determined to be 664.8 (calculated value: 664.77) by mass spectrometry.

Preparation of compound 8:

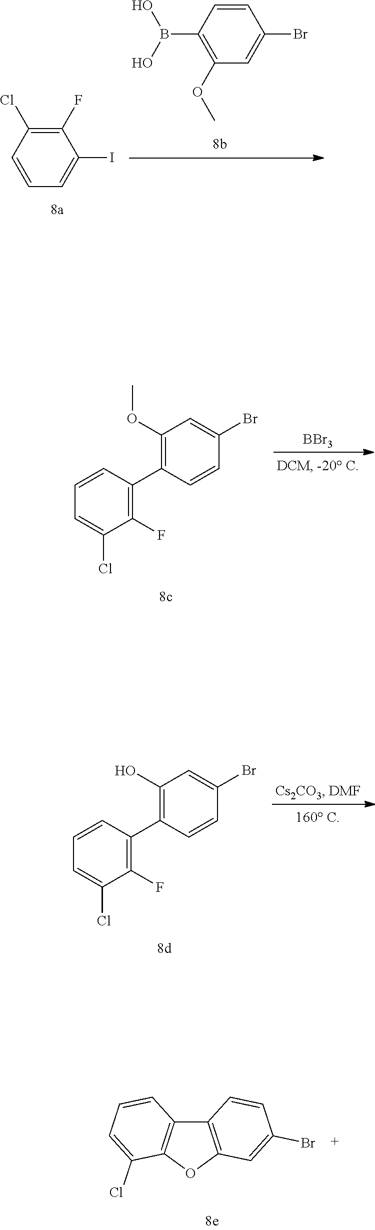

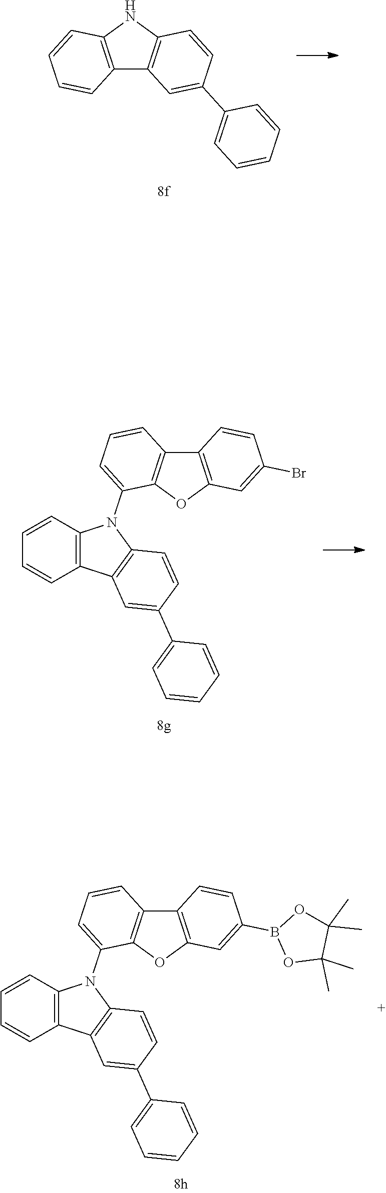 [SMILES: c1ccc(-c2ccc3[nH]c4ccccc4c3c2)cc1 ; Brc1ccc2c(c1)oc1c(-n3c4ccccc4c4cc(-c5ccccc5)ccc43)cccc12 ; CC1(C)OB(c2ccc3c(c2)oc2c(-n4c5ccccc5c5cc(-c6ccccc6)ccc54)cccc23)OC1(C)C]

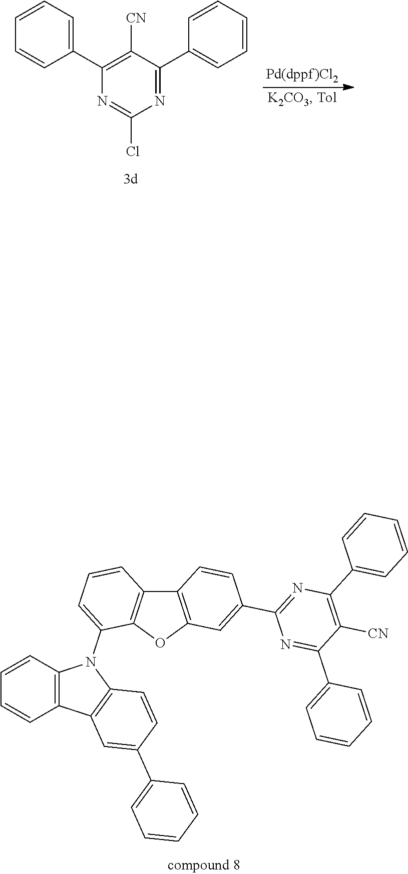 [SMILES: N#Cc1c(-c2ccccc2)nc(Cl)nc1-c1ccccc1 ; "K2CO3, Tol" ; N#Cc1c(-c2ccccc2)nc(-c2ccc3c(c2)oc2c(-n4c5ccccc5c5cc(-c6ccccc6)ccc54)cccc23)nc1-c1ccccc1]

8a (30 g), 8b (30 g), potassium carbonate (25 g), tetrakis(triphenylphosphine)palladium (1 g), and 300 mL of toluene/50 mL of ethanol/50 mL of water were added to a three-necked flask under N₂ atmosphere. After heating up to 100°° C., the resulting mixture was refluxed for 12 h. After the mixture was cooled, the resulting solution was extracted with ethyl acetate and deionized water, the organic phase was washed with water two times, dried, filtered, and the solvent was removed to obtain 26.3 g of the crude product (intermediate 8c). After that, the resulting sample was purified by column chromatography (petroleum ether: ethyl acetate=15:1) to yield 25.1 g (87% yield) of intermediate 8c. The molecular ion mass was determined to be 315.6 (calculated value: 315.57) by mass spectrometry.

Intermediate 8c (26.3 g) and dichloromethane (200 mL) were added to a dry three-necked flask, then the resulting mixture was cooled down to below 20° C. After adding 50 mL of boron tribromide dropwise to the reaction system, the resulting mixture was naturally heated to reflux for 12 h. After the reaction was completed, the resulting mixture was quenched with ethanol in an ice bath, and extracted with dichloromethane and deionized water. After washing with deionized water two times, the result was dried, filtered, and the solvent was removed to obtain 24.9 g of the crude product (8d) which was used directly in the next step of the reaction without any other treatment.

8d (24.9 g) was added to a single-necked flask, then N,N-dimethylformamide (300 mL) and cesium carbonate (25 g) were added. After heating up to 140° C., the resulting mixture was melted, stirred, and reacted for 12 h. After the mixture was cooled, the resulting mixture was extracted with deionized water and ethyl acetate, the organic phase was washed with water three times, dried to obtain 23.6 g of the crude product (8e). After that, the resulting sample was purified by column chromatography (petroleum ether: ethyl acetate=15:1) to yield 20.2 g (75% yield) of 8e. The molecular ion mass was determined to be 281.5 (calculated value: 281.53) by mass spectrometry.

8f (25 g), 8e (20.2 g), potassium carbonate (25 g), tetrakis(triphenylphosphine)palladium (1 g), and 300 mL of toluene/50 mL of ethanol/50 mL of water were added to a three-necked flask under N₂ atmosphere. After heating up to 100° C., the resulting mixture was refluxed for 12 h. After the mixture was cooled, the resulting solution was extracted with ethyl acetate and deionized water, the organic phase was washed with water two times, dried, filtered, and the solvent was removed to obtain 19.3 g of the crude product (8g). After that, the resulting sample was purified by column chromatography (petroleum ether: ethyl acetate=15:1) to yield 19.1 g (71% yield) of 8g. The molecular ion mass was determined to be 488.4 (calculated value: 488.38) by mass spectrometry.

8g (19.1 g), bis(pinacolato)diboron (25 g), potassium acetate (15 g), catalyst Pd(dppf)Cl₂ (1 g), and toluene (300 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 16 g (69% yield) of 8h. The molecular ion mass was determined to be 535.4 (calculated value: 535.45) by mass spectrometry.

Intermediate 3d (10 g), 8h (10 g), potassium acetate (10 g), catalyst Pd(dppf)Cl₂ (1 g), toluene (200 mL), ethanol (40 mL), and water (40 mL) were added to a dry three-necked flask under N₂ atmosphere. After heating up to 100° C., the resulting mixture was stirred and refluxed for 24 h. After the reaction was completed, the resulting sample was purified by column chromatography (petroleum ether: ethyl acetate=25:1) to yield 14.1 g (91% yield) of compound 8. The molecular ion mass was determined to be 664.8 (calculated value: 664.77) by mass spectrometry.
2. Energy Structure of Organic Compounds

The energy level of the organic material can be calculated by quantum computation, for example, using TD-DFT (time-dependent density functional theory) by Gaussian 03W (Gaussian Inc.), the specific simulation methods of which can be found in WO2011141110. Firstly, the molecular geometry is optimized by semi-empirical method “Ground State/Semi-empirical/Default Spin/AM1” (Charge 0/Spin Singlet), and then the energy structure of organic molecules is calculated by TD-DFT (time-dependent density functional theory) “TD-SCF/DFT/Default Spin/B3PW91” and the basis set “6-31G (d)” (Charge 0/Spin Singlet). The HOMO and LUMO energy levels are calculated using the following calibration formula, where S1 and T1 are used directly.

$$HOMO ( eV ) = ( ( HOMO ( G ) \times 27.212 ) - 0.9899 ) / 1.1206$$

$$LUMO ( eV ) = ( ( LUMO ( G ) \times 27.212 ) - 2.0041 ) / 1.385$$

Where HOMO (G) and LUMO (G) are the direct calculation results of Gaussian 03W, in units of Hartree. The results are shown in Table 1:

TABLE 1

| Materials | HOMO [eV] | LUMO [eV] | ΔHOMO [eV] | ΔLUMO [eV] | T1 [eV] | S1 [eV] |
|---|---|---|---|---|---|---|
| Compound1 | −5.52 | −3.08 | 0.43 | 0.24 | 2.43 | 2.45 |
| Compound 2 | −5.63 | −3.07 | 0.45 | 0.20 | 2.52 | 2.53 |
| Compound 3 | −5.63 | −3.13 | 0.45 | 0.28 | 2.42 | 2.46 |
| Compound 4 | −5.70 | −3.05 | 0.43 | 0.19 | 2.59 | 2.77 |
| Compound 5 | −5.72 | −3.07 | 0.45 | 0.21 | 2.53 | 2.73 |
| Compound 6 | −5.69 | −3.09 | 0.28 | 0.18 | 2.62 | 2.67 |
| Compound 7 | −5.77 | −3.09 | 0.34 | 0.31 | 2.56 | 2.79 |
| Compound 8 | −5.73 | −3.10 | 0.40 | 0.32 | 2.53 | 2.62 |
| PH-1 | −5.33 | −2.38 | 0.36 | 0.31 | 2.73 | 3.41 |
| PH-2 | −5.42 | −2.23 | 0.41 | 0.03 | 2.98 | 3.56 |

3. Preparation and Characterization of OLEDs

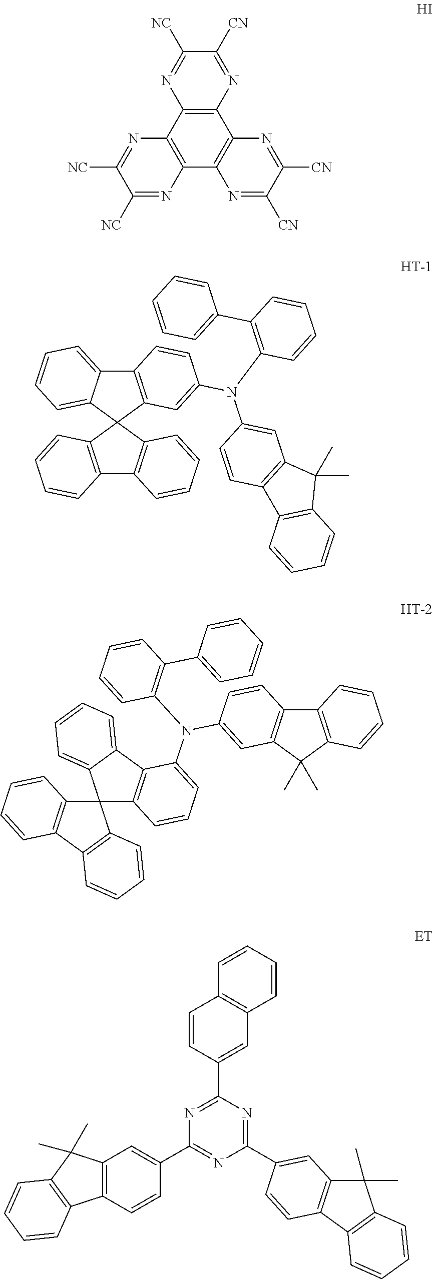 [SMILES: N#Cc1nc2c3nc(C#N)c(C#N)nc3c3nc(C#N)c(C#N)nc3c2nc1C#N ; CC1(C)c2ccccc2-c2ccc(N(c3ccc4c(c3)C3(c5ccccc5-c5ccccc53)c3ccccc3-4)c3ccccc3-c3ccccc3)cc21 ; CC1(C)c2ccccc2-c2ccc(N(c3ccccc3-c3ccccc3)c3cccc4c3-c3ccccc3C43c4ccccc4-c4ccccc43)cc21 ; CC1(C)c2ccccc2-c2ccc(-c3nc(-c4ccc5c(c4)C(C)(C)c4ccccc4-5)nc(-c4ccc5ccccc5c4)n3)cc21]

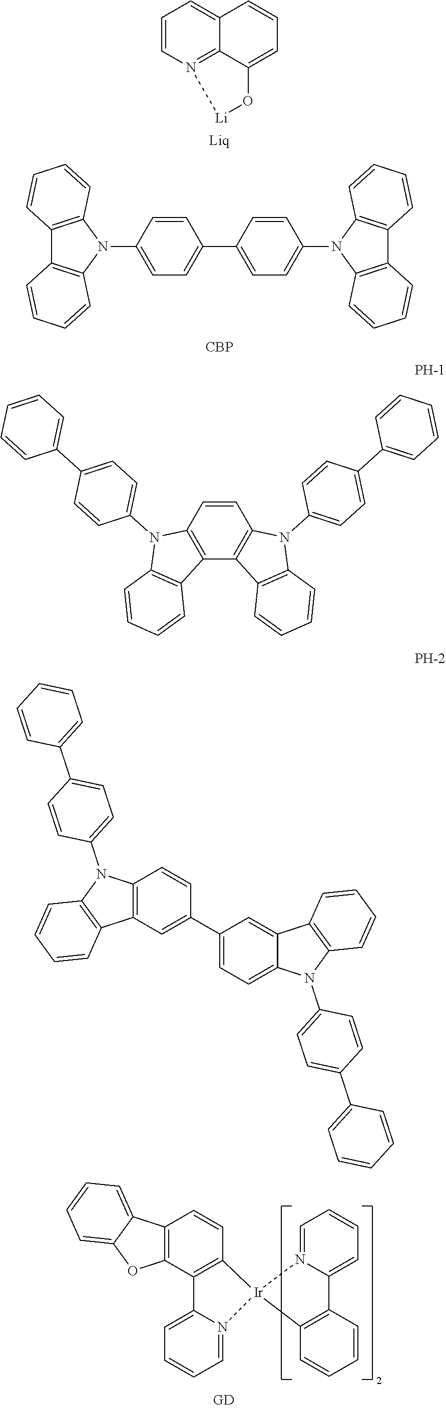

The preparation process of the OLEDs will be described in detail with reference to specified examples below. The structure of the green-emitting OLED is as follows: ITO/HI/HT-1/HT-2/EML/ET Liq/Liq/Al. The preparation steps of device example 1 are as follows:
a. Cleaning of the ITO (Indium Tin Oxide) conductive glass substrate: the substrates are washed with various solvents (such as one or more of chloroform, ketone, or isopropyl alcohol), and then treated with UV and ozone.
b. The materials were vacuum-deposited in high vacuum (1×10⁻⁶ mbar) to form the following layers: HI (30 nm), HT-1 (50 nm), HT-2 (10 nm), host material: 10% GD (40 nm), ET: Liq (50:50, 30 nm), Liq (1 nm), and Al (100 nm). After cleaning, the resultant ITO substrate was mounted on a vacuum deposition apparatus, and compound HI was then vacuum-deposited on the anode to form a hole-injection layer having a thickness of 30 nm, then two hole-transport layers, HT-1 (50 nm) and HT-2 (10 nm) were deposited on the HIL in vaccum deposition sequentially. Then compound 1 (host) and compound GD (dopant) in two different evaporation sources were deposited at a weight ratio of 100:10 to form a light-emitting layer having a thickness of 40 nm. Subsequently, compound ET and Liq were placed in two different evaporation sources, and co-deposited on the light-emitting layer at a weight ratio of 50:50 to form an electron-transport layer having a thickness of 30 nm. Liq was then deposited on the electron-transport layer to form an electron-injection layer having a thickness of 1 nm and Al was deposited on the electron-injection layer to form a cathode having a thickness of 100 nm.
c. Encapsulation: encapsulating the device in a nitrogen-regulated glove box with UV curable resin.

Device example 2-device example 20 are implemented in the same way as the device example 1, except that the compound 1 was replaced with different host and co-host combinations (where co-host means that the two compounds were separately placed in two different evaporation units, and the weight ratio of their materials was controlled to be 50:50).

TABLE 2

| | | CE (cd/A) | |
|---|---|---|---|
| OLED Devices | Hosts | @1000 nits | T95@1000 nits |
| Device Example 1 | Compound1 | 60.7 | 257% |
| Device Example 2 | Compound 2 | 61.0 | 268% |
| Device Example 3 | Compound 3 | 61.3 | 249% |
| Device Example 4 | Compound 4 | 60.5 | 273% |
| Device Example 5 | Compound 5 | 59.4 | 262% |
| Device Example 6 | Compound 6 | 59.7 | 250% |
| Device Example 7 | Compound 7 | 59.1 | 216% |
| Device Example 8 | Compound 8 | 58.8 | 234% |
| Device example 9 | Compound 1:PH- | 69.4 | 320% |
| | 1 = 5:5 | | |
| Device example 10 | Compound 2:PH- | 68.6 | 365% |
| | 1 = 5:5 | | |
| Device example 11 | Compound 3:PH- | 70.0 | 339% |
| | 1 = 5:5 | | |
| Device example 12 | Compound 4:PH- | 69.1 | 351% |
| | 1 = 5:5 | | |
| Device example 13 | Compound 5:PH- | 67.5 | 326% |
| | 1 = 5:5 | | |
| Device example 14 | Compound 7:PH- | 67.9 | 317% |
| | 1 = 5:5 | | |
| Device example 15 | Compound 1:PH- | 70.3 | 342% |
| | 2 = 5:5 | | |
| Device example 16 | Compound 2:PH- | 69.5 | 344% |
| | 2 = 5:5 | | |
| Device example 17 | Compound 3:PH- | 69.3 | 370% |
| | 2 = 5:5 | | |
| Device example 18 | Compound 4:PH- | 70.1 | 356% |
| | 2 = 5:5 | | |
| Device example 19 | Compound 5:PH- | 68.0 | 329% |
| | 2 = 5:5 | | |
| Device example 20 | Compound 7:PH- | 67.4 | 322% |
| | 2 = 5:5 | | |
| Comparative | CBP | 40.2 | 100% |
| example 1 | | | |

The current-voltage and luminescence (IVL) characteristics of the green-emitting OLEDs were studied. The efficiency, device lifetime, and driving voltage were summarized in table 2. The lifetime of each device example 1-device example 20 is a relative value of that of Comparative Example 1.

What is claimed is:

1. An organic compound, comprising a structure of formula (I): 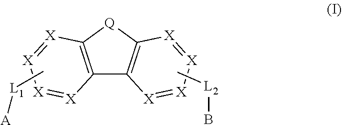
wherein,
Q is O or S;
Xs are the same or different at each occurrence, and denote CR₁ or N, wherein no more than two Xs in each ring are N atoms;
A comprises a structure of formula (A), B comprises a structure of formula (B): 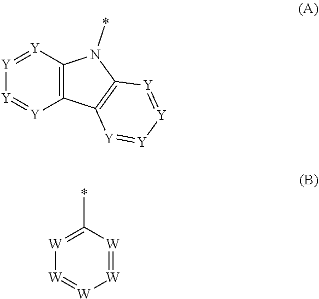
wherein * denotes a linkage site;
Ys are the same or different at each occurrence, and denote CR₂ or N, wherein no more than two Ys in each ring are N atoms, or the adjacent two Ys are groups represented by formula (I-1) and formula (I-2), respectively, while the remaining Ys are the same or different at each occurrence, and denote CR₁ or N; 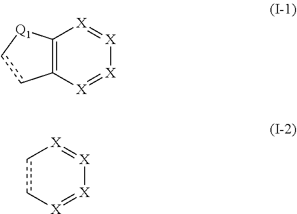
wherein each dashed bond denotes a bonding position of the group (i.e., the position of Y=Y or Y-Y in formula (A)), each X is defined as described herein, Q₁ is selected from O, S, NR₂, or CR₃R₄;
Ws are the same or different and independently be CR₅ or N, and at least one W is C—CN while at least two Ws are N atoms;
L₁ is a single bond, or a substituted/unsubstituted aromatic or heteroaromatic group containing 5 to 30 ring atoms;
L₂ is a single bond, or a substituted/unsubstituted aromatic or heteroaromatic group containing 5 to 30 ring atoms;
R₁ to R₅ are the same or different at each occurrence, and independently selected from the group consisting of —H, —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₁-R₅ form a monocyclic or polycyclic aliphatic or aromatic ring system with each other and/or with the rings bonded thereto.

2. The organic compound according to claim 1, wherein A is selected from the following structures: 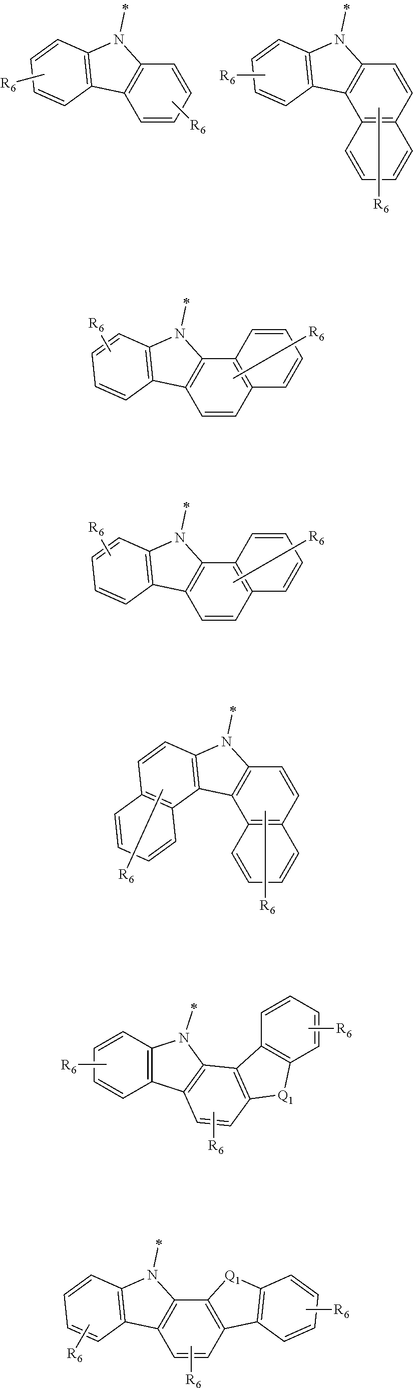 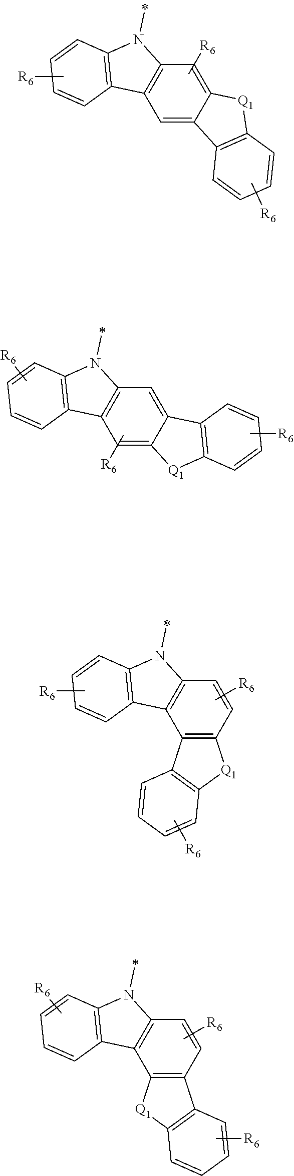
wherein, Q₁ is selected from O, S, NR₂, or CR₃R₄;
each R₆ is a substituent, and at each occurrence is independently selected from the group consisting of —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₆s form a monocyclic or polycyclic aliphatic or aromatic ring system with each other and/or with the rings bonded thereto.

3. The organic compound according to claim 1, wherein the organic compound is selected from the following structures of formulas (II-1)-(II-14): 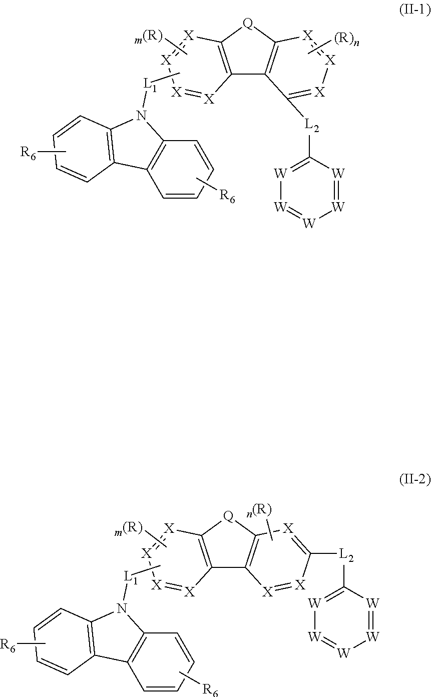 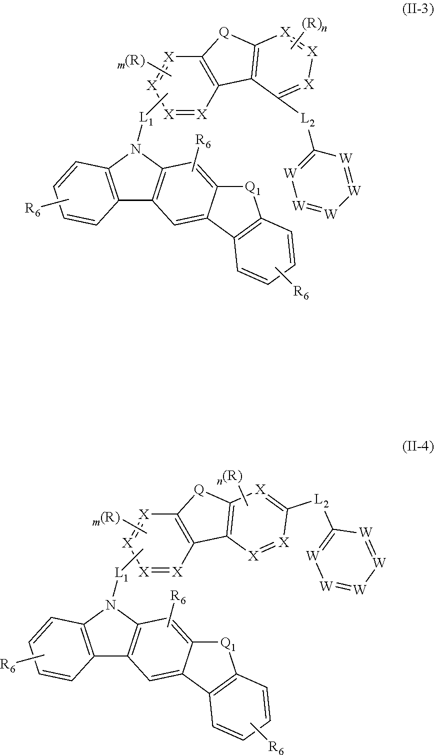 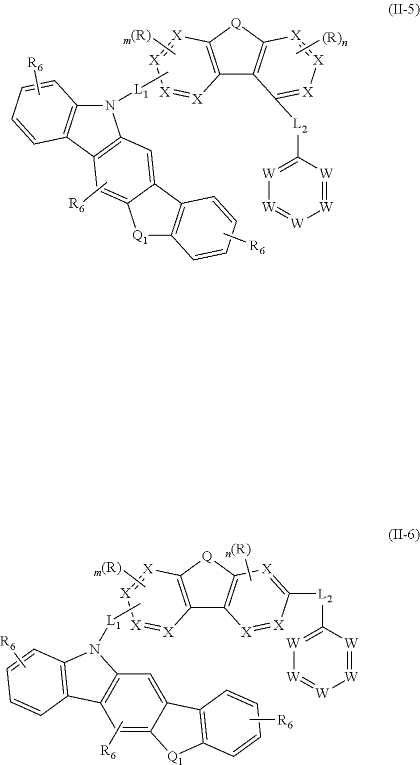 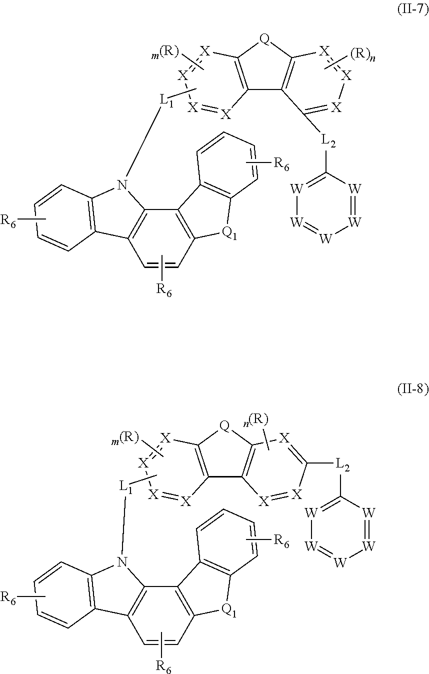 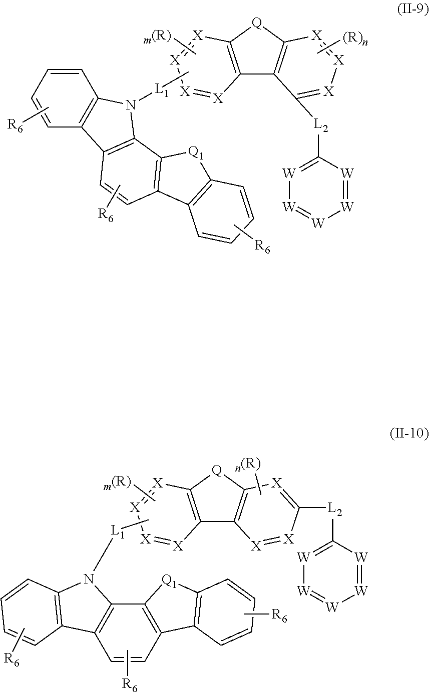 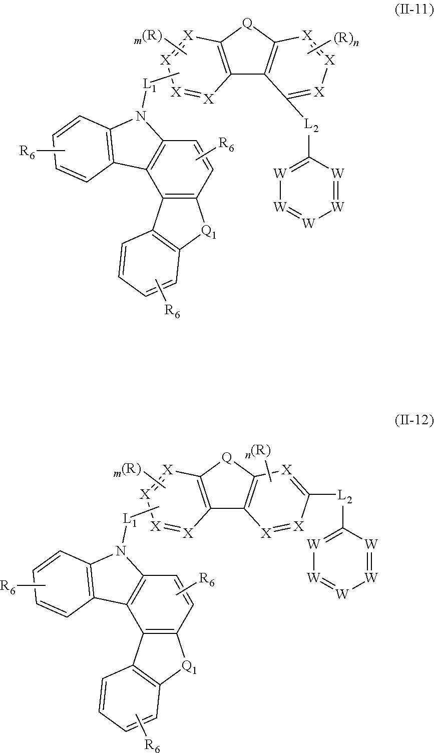 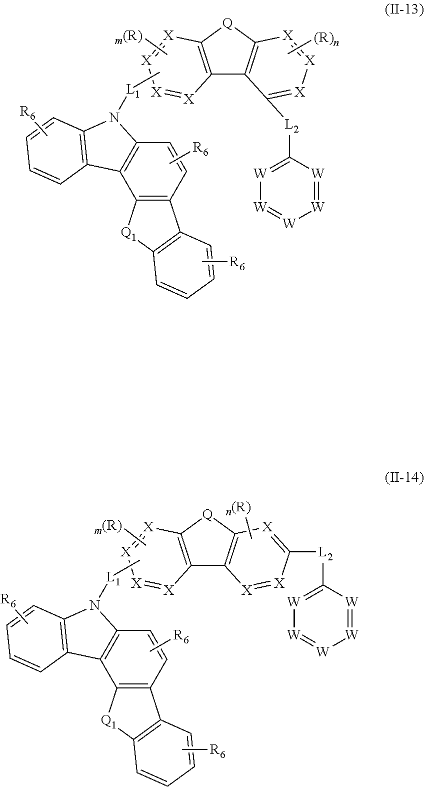
wherein, m and n are independently selected from integers from 0 to 3, each R is a substituent, and at each occurrence is independently selected from the group consisting of —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₆s form a monocyclic or polycyclic aliphatic or aromatic ring system with each other and/or with the rings bonded thereto.

4. The organic compound according to claim 2, wherein the organic compound is selected from the following structures of formulas (II-1)-(II-14): 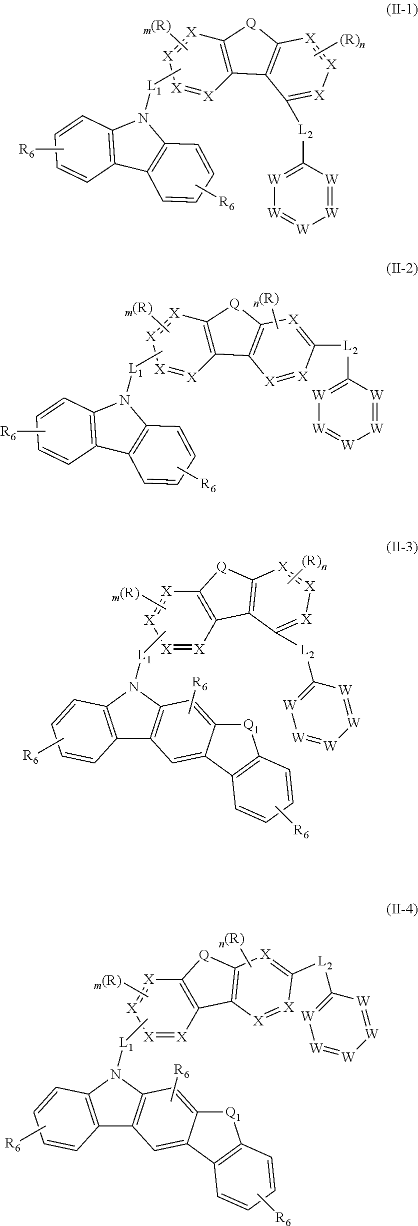 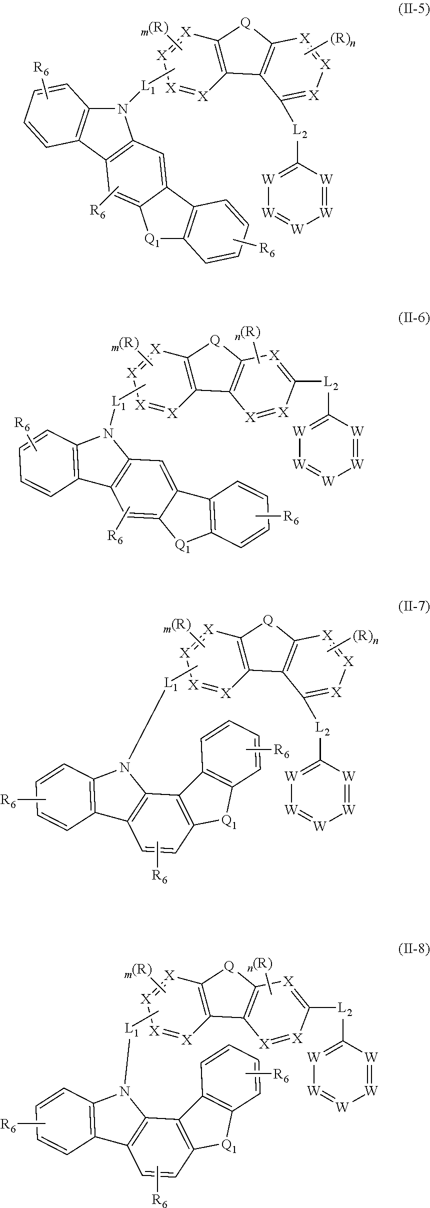 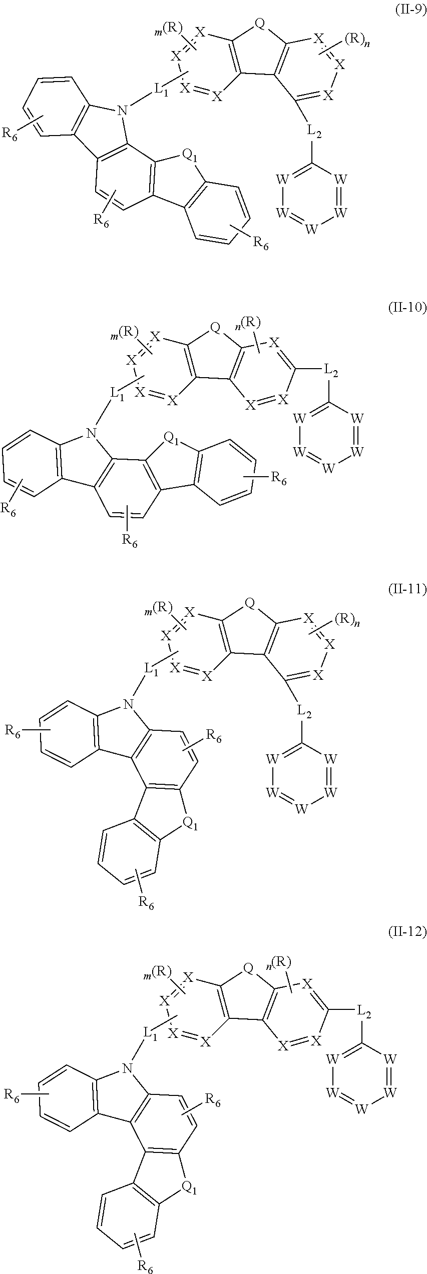 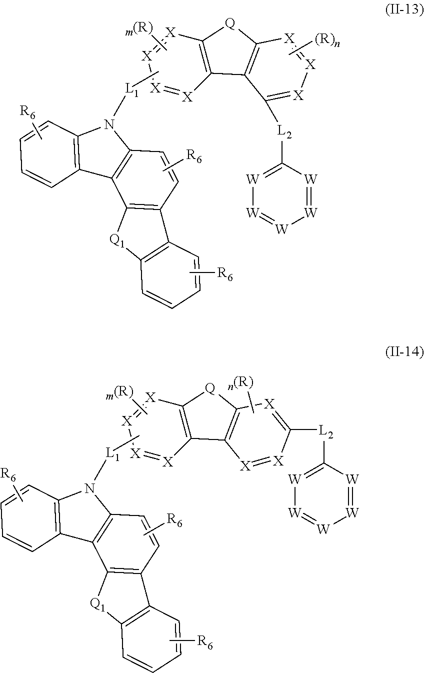
wherein, m and n are independently selected from integers from 0 to 3, each R is a substituent, and at each occurrence is independently selected from the group consisting of —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₆s form a monocyclic or polycyclic aliphatic or aromatic ring system with each other and/or with the rings bonded thereto.

5. The organic compound according to claim 1, wherein each of L₁ and L₂ is independently selected from the following groups or any combination thereof: 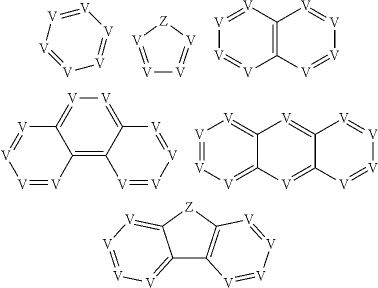
wherein:
V at each occurrence is CR¹ or N;
Z at each occurrence is independently selected from NR₇, CR₈R₉, O, S, SiR₁₀R₁₁, S═O, or SO₂;
each of R¹ and R₇-R₁₁ is a substituent, and at each occurrence is independently selected from the group consisting of —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₆s form a monocyclic or polycyclic aliphatic or aromatic ring system with each other and/or with the rings bonded thereto.

6. The organic compound according to claim 2, wherein each of L₁ and L₂ is independently selected from the following groups or any combination thereof: 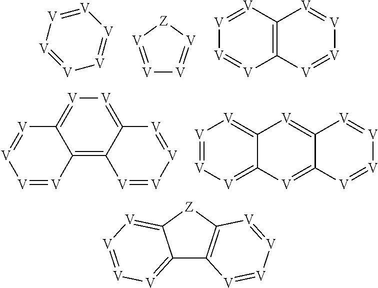
wherein:
V at each occurrence is CR¹ or N;
Z at each occurrence is independently selected from NR₇, CR₈R₉, O, S, SiR₁₀R₁₁, S═O, or SO₂;
each of R¹ and R₇-R₁₁ is a substituent, and at each occurrence is independently selected from the group consisting of —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more Res form a monocyclic or polycyclic aliphatic or aromatic ring system with each other and/or with the rings bonded thereto.

7. The organic compound according to claim 3, wherein each of L₁ and L₂ is independently selected from the following groups or any combination thereof: 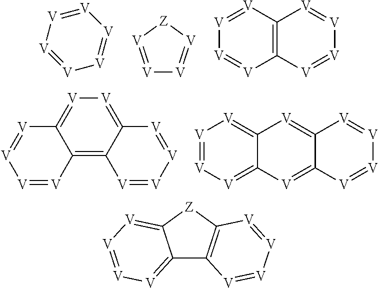
wherein:
V at each occurrence is CR¹ or N;
Z at each occurrence is independently selected from NR₇, CR₈R₉, O, S, SiR₁₀R₁₁, S═O, or SO₂;
each of R¹ and R₇-R₁₁ is a substituent, and at each occurrence is independently selected from the group consisting of —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₆s form a monocyclic or polycyclic aliphatic or aromatic ring system with each other and/or with the rings bonded thereto.

8. The organic compound according to claim 4, wherein each of L₁ and L₂ is independently selected from the following groups or any combination thereof: 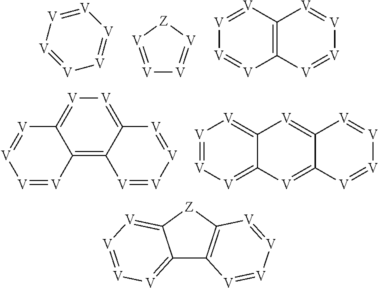
wherein:
V at each occurrence is CR¹ or N;
Z at each occurrence is independently selected from NR₇, CR₈R₉, O, S, SiR₁₀R₁₁, S═O, or SO₂;
each of R¹ and R₇-R₁₁ is a substituent, and at each occurrence is independently selected from the group consisting of —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₆s form a monocyclic or polycyclic aliphatic or aromatic ring system with each other and/or with the rings bonded thereto.

9. A mixture, comprising a first organic compound (H1) and a second organic compound (H2), wherein the first organic compound (H1) comprises at least one of the organic compound according to claim 1, the second organic compound (H2) has hole-transporting property, and the molar ratio of the first organic compound (H1) to the second organic compound (H2) ranges from 1:9 to 9:1.

10. The mixture according to claim 9, wherein the first organic compound (H1) and the second organic compound (H2) form type II heterojunction energy structure, the first organic compound (H1) comprises a structural unit of formula (I), and min(LUMO(H1)−HOMO(H2), LUMO(H2)−HOMO(H1))≤min(E_{T}(H1), E_{T}(H2))+0.1 eV, where LUMO(H1), HOMO(H1), and E_{T}(H1) respectively represent the lowest unoccupied orbital level, the highest occupied orbital level, and the triplet energy level of the first organic compound (H1); LUMO(H2), HOMO(H2), and E_{T}(H2) respectively represent the lowest unoccupied orbital level, the highest occupied orbital level, and the triplet energy level of the second organic compound (H2).

11. The mixture according to claim 9, wherein the second organic compound (H2) is a combination of formula (III-1), and formula (III-2) or formula (III-3): 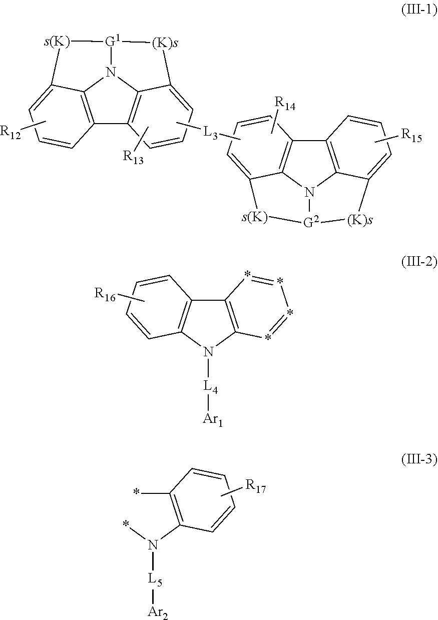
each of G₁ and G₂ at each occurrence is a substituted/unsubstituted aromatic or heteroaromatic group containing 5 to 30 ring atoms;
K at each occurrence is a single bond or CR₁₈R₁₉;
s at each occurrence is 0 or 1;
the adjacent two *s in formula (III-2) are linked to formula (III-3), and the unlinked *s in both formula (III-2) and formula (III-3) are CR₂₀s;
each of L₃ to L₅ is a single bond, or a substituted/unsubstituted aromatic or heteroaromatic group containing 5 to 30 ring atoms;
each of R₁₂ to R₁₇ is a substituent, and at each occurrence is independently selected from the group consisting of —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₆s form a monocyclic or polycyclic aliphatic or aromatic ring system with each other and/or with the rings bonded thereto; each of R₁₈ to R₂₀ are the same or different at each occurrence, and independently selected from the group consisting of —H, —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₁-R₅ form a monocyclic or polycyclic aliphatic or aromatic ring system with each other and/or with the rings bonded thereto;
each of Ar₁ and Ar₂ is a substituted/unsubstituted aromatic group containing 6 to 30 ring atom, or a substituted/unsubstituted heteroaromatic group containing 5 to 30 ring atom.

12. The mixture according to claim 11, wherein the second organic compound (H2) is a compound of formulas (IV-1)-(IV-6): 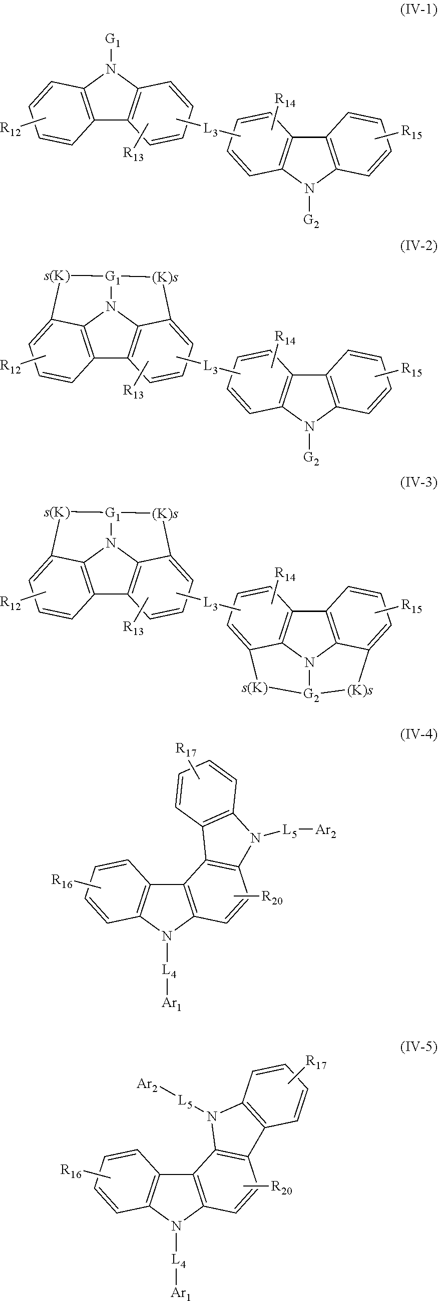 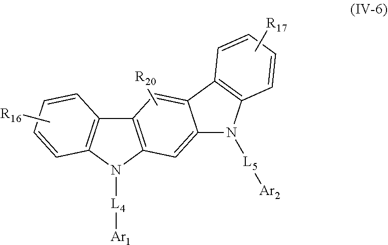
wherein, each of G₁ and G₂ at each occurrence is a substituted/unsubstituted aromatic or heteroaromatic group containing 5 to 30 ring atoms; K at each occurrence is a single bond or CR₁₈R₁₉; each of R₁₂ to R₁₇ is a substituent, and at each occurrence is independently selected from the group consisting of —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₆s form a monocyclic or polycyclic aliphatic or aromatic ring system with each other and/or with the rings bonded thereto; R₂₀s are same or different at each occurrence, and independently selected from the group consisting of —H, —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₁-R₅ form a monocyclic or polycyclic aliphatic or aromatic ring system with each other and/or with the rings bonded thereto; each of L₃ to L₅ is a single bond, or a substituted/unsubstituted aromatic or heteroaromatic group containing 5 to 30 ring atoms; each of Ar₁ and Ar₂ is a substituted/unsubstituted aromatic group containing 6 to 30 ring atom, or a substituted/unsubstituted heteroaromatic group containing 5 to 30 ring atom; s at each occurrence is 0 or 1.

13. The mixture according to claim 9, wherein the mixture further comprises an emitting material, the emitting material is selected from a singlet emitting material, a triplet emitting material, or a TADF material.

14. The mixture according to claim 10, wherein the mixture further comprises an emitting material, the emitting material is selected from a singlet emitting material, a triplet emitting material, or a TADF material.

15. An organic electronic device, comprising the organic compound according to claim 1.

16. The organic electronic device according to claim 15, wherein the organic electronic device is selected from an organic light-emitting diode, an organic photovoltaic cell, an organic light emitting electrochemical cell, an organic field effect transistor, an organic light emitting field effect transistor, an organic laser, an organic spintronic device, an organic sensor, or an organic plasmon emitting diode.

17. The organic electronic device according to claim 15, wherein the organic electronic device is an organic electroluminescent device comprising a light-emitting layer, the light-emitting layer comprises the organic compound.

18. An organic electronic device, comprising the mixture according to claim 9.

19. The organic electronic device according to claim 18, wherein the organic electronic device is selected from an organic light-emitting diode, an organic photovoltaic cell, an organic light emitting electrochemical cell, an organic field effect transistor, an organic light emitting field effect transistor, an organic laser, an organic spintronic device, an organic sensor, or an organic plasmon emitting diode.

20. The organic electronic device according to claim 18, wherein the organic electronic device is an organic electroluminescent device comprising a light-emitting layer, the light-emitting layer comprises the mixture.
